(19) United States
(12) Patent Application Publication
(10) Pub. No.: US 2025/0275560 A1
(43) Pub. Date: Sep. 4, 2025

(54) PROCESS OF MAKING GINGEROL COMPOUNDS AND THEIR USE AS FLAVOR MODIFIERS

(71) Applicant: Firmenich SA, Satigny (CH)

(72) Inventors: Andreas TAGLIEBER, Satigny (CH); Fredi BRUHLMANN, Satigny (CH)

(21) Appl. No.: 18/286,511

(22) PCT Filed: May 6, 2022
(86) PCT No.: PCT/EP22/62263

§ 371 Date: Oct. 11, 2023

(30) Foreign Application Priority Data
May 11, 2021 (EP) 21173202.9

(51) Int. Cl. A23L 27/20; A23L 27/00; C07C 67/08; C12N 9/04; C12P 7/22

(52) U.S. Cl. CPC A23L 27/204; A23L 27/88; C07C 67/08; C12N 9/0006; C12P 7/22; C12Y 101/01002

(57) ABSTRACT

Described herein are processes of preparing gingerol compounds, whether in their diol form or as a derivative, and methods of using gingerol compounds to enhance the salty taste or the umami taste of a flavored article. Also described herein are compositions and flavored products containing such compounds. In some embodiments, the flavored products have reduced amounts of salt or umami-enhancing compounds (glutamates, arginates, purinic ribonucleotides, such as inosine monophosphate (IMP) and guanosine monophosphate (GMP)), relative to comparable flavored products that do not incorporate the gingerol compounds.

### CROSS-REFERENCE TO RELATED APPLICATIONS

This application is a U.S. National Phase Application of International Patent Application No. PCT/EP22/62263, filed May 6, 2022, which claims the benefit of priority to European Patent Application No. 21173202.9, filed May 11, 2021, the entire contents of which are hereby incorporated by reference herein.

### INTRODUCTION

### Reference to an Electronic Sequence Listing

The contents of the electronic sequence listing (txt_36803_392_sequence_listing.txt; Size: 6,501 bytes; and Date of Creation: Mar. 8, 2024) are herein incorporated by reference in their entirety.

Table salt (sodium chloride), is a commonly used compound for eliciting the perception of salty taste in ingestible products, and has many desirable culinary properties to improve the sensory properties of food products and to enhance the positive sensory attributes of foods to taste better. Reduced sodium food may not taste good for people who are accustomed to high levels of salt in their food. Salt levels in food products may be reduced by understanding the flavor-enhancing properties and flavor perception of salts. The development of salt substitutes or enhancers can contribute to a reduction of salt in food products by understanding how salt is detected by sensory receptors. The taste of salt can also encompass the larger concept of flavor. The sense of taste is composed of a small number of primary or basic taste qualities, such as sweet, sour, salty, bitter, and savory or umami. Sodium chloride is the prototypical salt taste molecule imparting an almost pure salt taste. A critical attribute of salt taste is its hedonic or pleasantness dimension to overall food flavor. Salt was found to improve the perception of product thickness, enhance sweetness, mask metallic or chemical off-notes, and round out overall flavor while improving flavor intensity.

It is generally believed that the cations of salts serve as the agents for imparting the perceptual taste component, while the anions, in addition to contributing to tastes of their own, modify the perception of the taste of the cations. By way of example, sodium and lithium are believed to impart only salty tastes, while potassium and other alkaline earth cations produce both salty and bitter tastes. Among the anions commonly found in foods, the chloride ion is considered to be the least inhibitory to the salty taste, while the citrate anion is more inhibitory.

Sodium chloride imparts an almost pure salt taste. Attempts have been made to provide salty tasting compositions as a substitute for sodium chloride to give the same or a similar seasoning effect. Potassium chloride is often used in lowered-sodium formulations. However, potassium chloride tastes both salty and bitter, and this bitterness is a concern in replacing the sensory effects of sodium chloride. Ammonium chloride and other similar compounds have a bitter aftertaste. Neither of these compounds individually or in combination positively affects other taste modalities and tastes like sodium chloride.

Surh et al (1994) Life Sciences Vol 54 No 19, 321-326 reports the enzymatic reduction or 6-gingerol in a cell free-preparation of rat liver. However it is not clear whether one single enzyme or several enzymes contributed to the conversion of [6]-gingerol to the [6]-gingerdiol, as rat liver contains many different enzymes which could catalyse this reaction. Moreover this is a pharmacological study and is not relevant to commercial production methods of [6]-gingerdiol. Therefore, the rat liver system can't be considered a practical method for the preparation of [6]-gingerdiol whose stereo-structure was not clearly identified by the authors of that study.

EP 1649759 discloses a flavor enhancer composition comprising ginger powder. However no analysis of the powder is reported in this document and therefore it is unknown whether a compound of formula (III) is present in the composition.

Thus, there is a continuing need to develop new compounds, compositions, and processes to modify or enhance the taste of the food products by enhancing the saltiness of reduced sodium foods.

### SUMMARY

The present invention provides processes for making compounds, compositions, and processes for enhancing the salty or umami taste of food products without having to use as much of compounds like table salt, MSG, and the like.

An aspect of the invention provides a process for making a compound of formula (III):

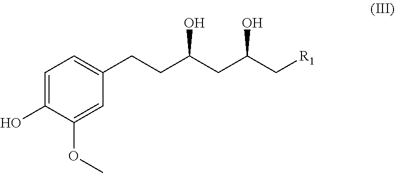

in the form of a mixture or salt thereof; wherein: R₁ is a hydrogen atom or a C₁₋₂₀ alkyl group.
the process comprising reducing a precursor compound of formula (II)

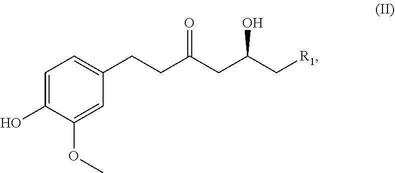

in the form of a mixture or salt thereof, wherein R₁ is defined as in formula (III), with an oxidoreductase to form a compound of formula (III).

An embodiment of the process of the invention is wherein the oxidoreductase is a carbonyl reductase and/or ketoreductase (KRED) enzyme.

An embodiment of the process of the invention is wherein the KRED enzyme has at least 50%, 55%, 60%, 65%, 70%, 75%, 80%, 85%, 90%, 95%, 96%, 97%, 98% or 99% sequence identity to SEQ ID NO: 1 or SEQ ID NO: 3 or comprises the amino acid sequence of SEQ ID NO: 1 or SEQ ID NO: 3, or the KRED enzyme IEP Ox95.

An embodiment of the process of the invention is wherein the ketoreductase reduction is performed in the presence of a co-factor; preferably the co-factor is NAD(P)H or NAD(P).

An embodiment of the process of the invention is wherein the ketoreductase reduction is performed in the presence of a co-factor regeneration system.

An embodiment of the process of the invention is wherein the co-factor regeneration system is a secondary alcohol (preferably isopropanol), a formate dehydrogenase (FDH), or by a glucose dehydrogenase (GDH) system.

An embodiment of the process of the invention wherein precursor compound of formula (II) is [6]-gingerol, [8]-gingerol, or [10]-gingerol; preferably [6]-gingerol.

An embodiment of the process of the invention wherein compound of formula (III) is—[6]-gingerdiol, [8]-gingerdiol, or [10]-gingerdiol; preferably [6]-gingerdiol.

An embodiment of the process of the invention wherein the process further comprises esterifying the compound of formula (III) to form a compound of formula (I),

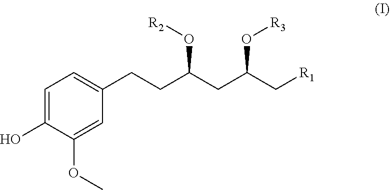

in the form of a mixture or salt thereof; wherein:
R₁ is a hydrogen atom or a C₁₋₂₀ alkyl group; and
each R₂ and R₃, are, independently from each other, —C(O)—H or —C(O)—(C₁₋₄ alkyl);

An embodiment of the process of the invention wherein the esterification of the compound of formula (III) to form a compound of formula (I) comprises using acetic acid. Preferably the esterification uses acetic acid.

A further aspect of the invention is a process for making a compound of formula (I) the process comprising reducing a precursor compound of formula (II) with a oxidoreductase enzyme to form a compound of formula (III), and the esterification of the compound of formula (III) to form a compound of formula (I).

An embodiment of the process of the invention is wherein the oxidoreductase is a ketoreductase (KRED) enzyme.

A further aspect of the invention provides a compound of formula (I) obtained or obtainable by the process of the invention

A further aspect of the invention is the use of a compound of formula (I) obtained or obtainable by the process of the invention to enhance a salty taste or an umami taste of a flavored product.

An embodiment of the invention is wherein the flavored product is a food product, a beverage product, or an oral care product.

An aspect of the invention is a method of enhancing a salty taste or an umami taste of a flavored product, the method comprising introducing to the food product a compound of formula (I) obtained or obtainable by the process of the invention.

An embodiment of the invention is wherein the flavored product is a food product, a beverage product, or an oral care product.

An aspect of the invention is a comestible composition for enhancing a salty taste or an umami taste in a flavored product, the composition comprising a compound of formula (I) obtained or obtainable by the process of the invention.

An aspect of the invention is a flavored product, which comprises a comestible composition of the invention which, in some embodiments, is a food product, a beverage product, or an oral care product.

Also provided herein is an isolated polypeptide having oxidoreductase activity comprising an amino acid sequence having at least 50%, 55%, 60%, 65%, 70%, 75%, 80%, 85%, 90%, 95%, 96%, 97%, 98% or 99% sequence identity to SEQ ID NO: 1 or 3 or comprising the amino acid sequence of SEQ ID NO: 1 or 3.

Further provided herein is an isolated nucleic acid molecule comprising a nucleotide sequence encoding a polypeptide having oxidoreductase activity and comprising an amino acid sequence having at least 50%, 55%, 60%, 65%, 70%, 75%, 80%, 85%, 90%, 95%, 96%, 97%, 98%, or 99% sequence identity to SEQ ID NO: 1 or 3 or comprising the amino acid sequence of SEQ ID NO: 1 or 3 or the reverse complement thereof.

Further provided is an isolated nucleic acid comprising a nucleotide sequence having at least 75%, 80%, 85%, 90%, 95%, 96%, 97%, 98% or 99% sequence identity to SEQ ID NO: 2 or SEQ ID NO: 4 or comprising the nucleotide sequence of SEQ ID NO: 2 and SEQ ID NO: 4 or the reverse complement thereof.

Further provided is an isolated nucleic acid molecule encoding a polypeptide provided herein.

In one aspect provided herein is a vector comprising the nucleic acid molecules described herein. In another aspect, the vector is an expression vector. In a further aspect, the vector is a prokaryotic vector, viral vector or a eukaryotic vector.

Also provided is a non-human host organism or a host cell comprising (1) a nucleic acid molecule described above, or (2) an expression vector comprising said nucleic acid molecule. In one aspect the non-human organism or host cell is a prokaryotic or eukaryotic cell. In another aspect the host cell is a bacterial cell, a plant cell, a fungal cell or a yeast. In a further aspect, the bacterial cell is E. coli and the yeast cell is Saccharomyces cerevisiae

Further provided is the use of a polypeptide described herein for producing a compound of formula (III):

### BRIEF DESCRIPTION OF THE DRAWINGS

FIG. 1: Biocatalytic reduction of a low content (S)-[6]-gingerol (32% purity) under non-optimized reaction conditions. (S)-[6]-gingerol (●), (R,S)-[6]-gingerdiol (♦), 8-gingerol (∘), 8-gingerdiol (⋄), 10-gingerol (−), 10-gingerdiol (▪)

FIG. 2: Non-catalyzed esterification of [6]-gingerdiol with acetic acid at 100° C.

FIG. 3 Non-catalyzed esterification of [6]-gingerdiol with acetic acid under boiling conditions.

FIG. 4: Acid catalyzed esterification

### DETAILED DESCRIPTION

The following Detailed Description sets forth various aspects and embodiments provided herein. The description is to be read from the perspective of the person of ordinary skill in the relevant art. Therefore, information that is well known to such ordinarily skilled artisans is not necessarily included.

### Abbreviations Used

bp base pair
kb kilo base
DNA deoxyribonucleic acid
cDNA complementary DNA
DTT dithiothreitol
GC gas chromatograph
IPTG isopropyl-D-thiogalacto-pyranoside
LB lysogeny broth
MS mass spectrometer/mass spectrometry
PCR polymerase chain reaction
RNA ribonucleic acid
mRNAmessenger ribonucleic acid
miRNA micro RNA
SiRNA small interfering RNA
rRNA ribosomal RNA
tRNA transfer RNA

### Definitions

The following terms and phrases have the meanings indicated below, unless otherwise provided herein. This disclosure may employ other terms and phrases not expressly defined herein. Such other terms and phrases have the meanings that they would possess within the context of this disclosure to those of ordinary skill in the art. In some instances, a term or phrase may be defined in the singular or plural. In such instances, it is understood that any term in the singular may include its plural counterpart and vice versa, unless expressly indicated to the contrary

As used herein, “Cₐ to C_{b}” or “C_{a-b}” in which “a” and “b” are integers, refer to the number of carbon atoms in the specified group. That is, the group can contain from “a” to “b”, inclusive, carbon atoms. Thus, for example, a “C₁ to C₄ alkyl” or “C₁₋₄ alkyl” group refers to all alkyl groups having from 1 to 4 carbons, that is, CH₃—, CH₃CH₂—, CH₃CH₂CH₂—, (CH₃)₂CH—, CH₃CH₂CH₂CH₂—, CH₃CH₂CH(CH₃)— and (CH₃)₃C—.

As used herein, “alkyl” means a straight or branched hydrocarbon chain that is fully saturated (i.e., contains no double or triple bonds). In some embodiments, an alkyl group has 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkyl” where no numerical range is designated). The alkyl group may also be a medium size alkyl having 1 to 9 carbon atoms. The alkyl group could also be a lower alkyl having 1 to 4 carbon atoms. The alkyl group may be designated as “C₁₋₄ alkyl” or similar designations. By way of example only, “C₁₋₄ alkyl” indicates that there are one to four carbon atoms in the alkyl chain, i.e., the alkyl chain is selected from the group consisting of methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl, hexyl, and the like. Unless indicated to the contrary, the term “alkyl” refers to a group that is not further substituted.

It is to be understood that certain radical naming conventions can include either a mono-radical or a di-radical, depending on the context. For example, where a substituent requires two points of attachment to the rest of the molecule, it is understood that the substituent is a di-radical. For example, a substituent identified as alkyl that requires two points of attachment includes di-radicals such as —CH₂—, —CH₂CH₂—, —CH₂CH(CH₃)CH₂—, and the like.

Wherever a substituent is depicted as a di-radical (i.e., has two points of attachment to the rest of the molecule), it is to be understood that the substituent can be attached in any directional configuration unless otherwise indicated. Thus, for example, a substituent depicted as -AE- or

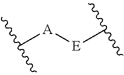

includes the substituent being oriented such that the A is attached at the leftmost attachment point of the molecule as well as the case in which A is attached at the rightmost attachment point of the molecule.

As used herein, the singular forms “a,” “an,” and “the” include plural referents unless the context clearly dictates otherwise. For example, reference to “a substituent” encompasses a single substituent as well as two or more substituents, and the like.

As used herein, “for example,” “for instance,” “such as,” or “including” are meant to introduce examples that further clarify more general subject matter. Unless otherwise expressly indicated, such examples are provided only as an aid for understanding embodiments illustrated in the present disclosure, and are not meant to be limiting in any fashion. Nor do these phrases indicate any kind of preference for the disclosed embodiment.

As used herein, “comprise” or “comprises” or “comprising” or “comprised of” refer to groups that are open, meaning that the group can include additional members in addition to those expressly recited. For example, the phrase, “comprises A” means that A must be present, but that other members can be present too. The terms “include,” “have,” and “composed of” and their grammatical variants have the same meaning. In contrast, “consist of” or “consists of” or “consisting of” refer to groups that are closed. For example, the phrase “consists of A” means that A and only A is present.

As used herein, “optionally” means that the subsequently described event(s) may or may not occur. In some embodiments, the optional event does not occur. In some other embodiments, the optional event does occur one or more times.

As used herein, “or” is to be given its broadest reasonable interpretation, and is not to be limited to an either/or construction. Thus, the phrase “comprising A or B” means that A can be present and not B, or that B is present and not A, or that A and B are both present. Further, if A, for example, defines a class that can have multiple members, e.g., A₁ and A₂, then one or more members of the class can be present concurrently.

As used herein, certain substituents or linking groups having only a single atom may be referred to by the name of the atom. For example, in some cases, the substituent “—H” may be referred to as “hydrogen” or “a hydrogen atom,” the substituent “—F” may be referred to as “fluorine” or “a fluorine atom,” and the linking group “—O—” may be referred to as “oxygen” or “an oxygen atom.”

Points of attachment for groups are generally indicated by a terminal dash (—) or by an asterisk (*). For example, a group such as *—CH₂—CH₃ or —CH₂—CH₃ both represent an ethyl group.

Chemical structures are often shown using the “skeletal” format, such that carbon atoms are not explicitly shown, and hydrogen atoms attached to carbon atoms are omitted entirely. For example, the structure

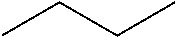

represents butane (i.e., n-butane). Furthermore, aromatic groups, such as benzene, are represented by showing one of the contributing resonance structures. For example, the structure

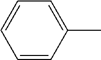

represents toluene.

As used herein, the term “gingerdiol compound” refers to compounds of formula (I), any salts thereof, or any generic or specific embodiments thereof.

As used herein a “salty taste modulating compound” is a compound that, when ingested, (i) elicits or enhances a perception of salty taste alone or in the presence of a salt, such as sodium chloride or (ii) alters the flow of ions through one or more ion channel associated with perception of salty taste. Examples of ion channels associated with the perception of salty taste include the ENaC channel, the TrpV1 channel and the TrpML3 channel.

Other terms are defined in other portions of this description, even though not included in this subsection.

The term “polypeptide” means an amino acid sequence of consecutively polymerized amino acid residues, for instance, at least 15 residues, at least 30 residues, at least 50 residues. In some embodiments herein, a polypeptide comprises an amino acid sequence that is an enzyme, or a fragment, or a variant thereof.

The term “protein” refers to an amino acid sequence of any length wherein amino acids are linked by covalent peptide bonds, and includes oligopeptide, peptide, polypeptide and full length protein whether naturally occurring or synthetic.

The term “isolated” polypeptide refers to an amino acid sequence that is removed from its natural environment by any method or combination of methods known in the art and includes recombinant, biochemical and synthetic methods.

The terms “biological function,” “function,” “biological activity” or “activity” refer to the ability of the drimenol synthase to catalyze the formation of drimenol or a mixture of compounds comprising drimenol and one or more terpenes.

The terms “nucleic acid sequence,” “nucleic acid,” “nucleic acid molecule” and “polynucleotide” are used interchangeably meaning a sequence of nucleotides. A nucleic acid sequence may be a single-stranded or double-stranded deoxyribonucleotide, or ribonucleotide of any length, and include coding and non-coding sequences of a gene, exons, introns, sense and anti-sense complimentary sequences, genomic DNA, cDNA, miRNA, siRNA, mRNA, rRNA, tRNA, recombinant nucleic acid sequences, isolated and purified naturally occurring DNA and/or RNA sequences, synthetic DNA and RNA sequences, fragments, primers and nucleic acid probes. The skilled artisan is aware that the nucleic acid sequences of RNA are identical to the DNA sequences with the difference of thymine (T) being replaced by uracil (U). The term “nucleotide sequence” should also be understood as comprising a polynucleotide molecule or an oligonucleotide molecule in the form of a separate fragment or as a component of a larger nucleic acid.

An “isolated nucleic acid” or “isolated nucleic acid sequence” relates to a nucleic acid or nucleic acid sequence that is in an environment different from that in which the nucleic acid or nucleic acid sequence naturally occurs and can include those that are substantially free from contaminating endogenous material. The term “naturally-occurring” as used herein as applied to a nucleic acid refers to a nucleic acid that is found in a cell of an organism in nature and which has not been intentionally modified by a human in the laboratory.

“Recombinant nucleic acid sequences” are nucleic acid sequences that result from the use of laboratory methods (for example, molecular cloning) to bring together genetic material from more than on source, creating or modifying a nucleic acid sequence that does not occur naturally and would not be otherwise found in biological organisms.

“Recombinant DNA technology” refers to molecular biology procedures to prepare a recombinant nucleic acid sequence as described, for instance, in Laboratory Manuals edited by Weigel and Glazebrook, 2002, Cold Spring Harbor Lab Press; and Sambrook et al., 1989, Cold Spring Harbor, NY, Cold Spring Harbor Laboratory Press.

The term “gene” means a DNA sequence comprising a region, which is transcribed into a RNA molecule, e.g., an mRNA in a cell, operably linked to suitable regulatory regions, e.g., a promoter. A gene may thus comprise several operably linked sequences, such as a promoter, a 5′ leader sequence comprising, e.g., sequences involved in translation initiation, a coding region of cDNA or genomic DNA, introns, exons, and/or a 3′non-translated sequence comprising, e.g., transcription termination sites.

A “chimeric gene” refers to any gene which is not normally found in nature in a species, in particular, a gene in which one or more parts of the nucleic acid sequence are present that are not associated with each other in nature. For example the promoter is not associated in nature with part or all of the transcribed region or with another regulatory region. The term “chimeric gene” is understood to include expression constructs in which a promoter or transcription regulatory sequence is operably linked to one or more coding sequences or to an antisense, i.e., reverse complement of the sense strand, or inverted repeat sequence (sense and antisense, whereby the RNA transcript forms double stranded RNA upon transcription). The term “chimeric gene” also includes genes obtained through the combination of portions of one or more coding sequences to produce a new gene.

A “3′ UTR” or “3′ non-translated sequence” (also referred to as “3′ untranslated region,” or “3′end”) refers to the nucleic acid sequence found downstream of the coding sequence of a gene, which comprises, for example, a transcription termination site and (in most, but not all eukaryotic mRNAs) a polyadenylation signal such as AAUAAA or variants thereof. After termination of transcription, the mRNA transcript may be cleaved downstream of the polyadenylation signal and a poly(A) tail may be added, which is involved in the transport of the mRNA to the site of translation, e.g., cytoplasm.

“Expression of a gene” encompasses “heterologous expression” and “over-expression” and involves transcription of the gene and translation of the mRNA into a protein. Overexpression refers to the production of the gene product as measured by levels of mRNA, polypeptide and/or enzyme activity in transgenic cells or organisms that exceeds levels of production in non-transformed cells or organisms of a similar genetic background.

“Expression vector” as used herein means a nucleic acid molecule engineered using molecular biology methods and recombinant DNA technology for delivery of foreign or exogenous DNA into a host cell. The expression vector typically includes sequences required for proper transcription of the nucleotide sequence. The coding region usually codes for a protein of interest but may also code for an RNA, e.g., an antisense RNA, siRNA and the like.

An “expression vector” as used herein includes any linear or circular recombinant vector including but not limited to viral vectors, bacteriophages and plasmids. The skilled person is capable of selecting a suitable vector according to the expression system. In one embodiment, the expression vector includes the nucleic acid of an embodiment herein operably linked to at least one regulatory sequence, which controls transcription, translation, initiation and termination, such as a transcriptional promoter, operator or enhancer, or an mRNA ribosomal binding site and, optionally, including at least one selection marker. Nucleotide sequences are “operably linked” when the regulatory sequence functionally relates to the nucleic acid of an embodiment herein.

“Regulatory sequence” refers to a nucleic acid sequence that determines expression level of the nucleic acid sequences of an embodiment herein and is capable of regulating the rate of transcription of the nucleic acid sequence operably linked to the regulatory sequence. Regulatory sequences comprise promoters, enhancers, transcription factors, promoter elements and the like.

“Promoter” refers to a nucleic acid sequence that controls the expression of a coding sequence by providing a binding site for RNA polymerase and other factors required for proper transcription including without limitation transcription factor binding sites, repressor and activator protein binding sites. The meaning of the term promoter also includes the term “promoter regulatory sequence”. Promoter regulatory sequences may include upstream and downstream elements that may influences transcription, RNA processing or stability of the associated coding nucleic acid sequence. Promoters include naturally-derived and synthetic sequences. The coding nucleic acid sequences is usually located downstream of the promoter with respect to the direction of the transcription starting at the transcription initiation site.

The term “constitutive promoter” refers to an unregulated promoter that allows for continual transcription of the nucleic acid sequence it is operably linked to.

As used herein, the term “operably linked” refers to a linkage of polynucleotide elements in a functional relationship. A nucleic acid is “operably linked” when it is placed into a functional relationship with another nucleic acid sequence. For instance, a promoter, or rather a transcription regulatory sequence, is operably linked to a coding sequence if it affects the transcription of the coding sequence. Operably linked means that the DNA sequences being linked are typically contiguous. The nucleotide sequence associated with the promoter sequence may be of homologous or heterologous origin with respect to the plant to be transformed. The sequence also may be entirely or partially synthetic. Regardless of the origin, the nucleic acid sequence associated with the promoter sequence will be expressed or silenced in accordance with promoter properties to which it is linked after binding to the polypeptide of an embodiment herein. The associated nucleic acid may code for a protein that is desired to be expressed or suppressed throughout the organism at all times or, alternatively, at a specific time or in specific tissues, cells, or cell compartment. Such nucleotide sequences particularly encode proteins conferring desirable phenotypic traits to the host cells or organism altered or transformed therewith. More particularly, the associated nucleotide sequence leads to the production of drimenol or a mixture comprising drimenol and one or more terpenes in the cell or organism. Particularly, the nucleotide sequence encodes a polypeptide having drimenol synthase activity.

“Target peptide” refers to an amino acid sequence which targets a protein, or polypeptide to intracellular organelles, i.e., mitochondria, or plastids, or to the extracellular space (secretion signal peptide). A nucleic acid sequence encoding a target peptide may be fused to the nucleic acid sequence encoding the amino terminal end, e.g., N-terminal end, of the protein or polypeptide, or may be used to replace a native targeting polypeptide.

The term “primer” refers to a short nucleic acid sequence that is hybridized to a template nucleic acid sequence and is used for polymerization of a nucleic acid sequence complementary to the template.

As used herein, the term “host cell” or “transformed cell” refers to a cell (or organism) altered to harbor at least one nucleic acid molecule, for instance, a recombinant gene encoding a desired protein or nucleic acid sequence which upon transcription yields a drimenol synthase protein useful to produce drimenol or a mixture comprising drimenol and one or more terpenes. The host cell is particularly a bacterial cell, a fungal cell or a plant cell. The host cell may contain a recombinant gene which has been integrated into the nuclear or organelle genomes of the host cell. Alternatively, the host may contain the recombinant gene extra-chromosomally.

Homologous sequences include orthologous or paralogous sequences. Methods of identifying orthologs or paralogs including phylogenetic methods, sequence similarity and hybridization methods are known in the art and are described herein.

Paralogs result from gene duplication that gives rise to two or more genes with similar sequences and similar functions. Paralogs typically cluster together and are formed by duplications of genes within related plant species. Paralogs are found in groups of similar genes using pair-wise Blast analysis or during phylogenetic analysis of gene families using programs such as CLUSTAL. In paralogs, consensus sequences can be identified characteristic to sequences within related genes and having similar functions of the genes.

Orthologs, or orthologous sequences, are sequences similar to each other because they are found in species that descended from a common ancestor. For instance, plant species that have common ancestors are known to contain many enzymes that have similar sequences and functions. The skilled artisan can identify orthologous sequences and predict the functions of the orthologs, for example, by constructing a polygenic tree for a gene family of one species using CLUSTAL or BLAST programs. A method for identifying or confirming similar functions among homologous sequences is by comparing of the transcript profiles in host cells or organisms, such as plants, overexpressing or lacking (in knockouts/knockdowns) related polypeptides. The skilled person will understand that genes having similar transcript profiles, with greater than 50% regulated transcripts in common, or with greater than 70% regulated transcripts in common, or greater than 90% regulated transcripts in common will have similar functions. Homologs, paralogs, orthologs and any other variants of the sequences herein are expected to function in a similar manner by making the host cells, organism such as plants producing drimenol synthase proteins.

The term “selectable marker” refers to any gene which upon expression may be used to select a cell or cells that include the selectable marker. Examples of selectable markers are described below. The skilled artisan will know that different antibiotic, fungicide, auxotrophic or herbicide selectable markers are applicable to different target species.

The term “organism” refers to any non-human multicellular or unicellular organisms such as a plant, or a microorganism. Particularly, a micro-organism is a bacterium, a yeast, an algae or a fungus.

The term “plant” is used interchangeably to include plant cells including plant protoplasts, plant tissues, plant cell tissue cultures giving rise to regenerated plants, or parts of plants, or plant organs such as roots, stems, leaves, flowers, pollen, ovules, embryos, fruits and the like. Any plant can be used to carry out the methods of an embodiment herein.

For the descriptions herein and the appended claims, the use of “or” means “and/or” unless stated otherwise. Similarly, “comprise,” “comprises,” “comprising”, “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

### Gingerol Compounds

In at least one aspect, the disclosure provides a process for making compounds of formula (I)

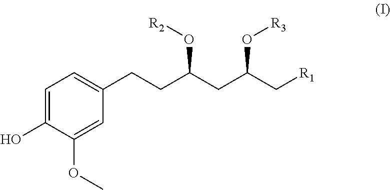

in the form of a mixture or salt thereof, wherein: R₁ is a hydrogen atom or a C₁₋₂₀ alkyl group; and each of R₂ and R₃ are, independently from each other, a hydrogen atom, —C(O)—H, or —C(O)—(C₁₋₂₀ alkyl).

The variable R₁ can have any suitable value consistent with the definitions set forth above. In some embodiments, R₁ is a C₅₋₁₂ alkyl group. In some embodiments, R₁ is a C₆₋₁₂ alkyl group. In some embodiments, R₁ is butyl. In some embodiments, R₁ is hexyl. In some embodiments, R₁ is octyl. In some embodiments, R₁ is decyl.

The variables R₂ and R₃ can have any suitable values consistent with the definition set forth above. In some embodiments, both R₂ and R₅ are a hydrogen atom. In some embodiments, one of R₂ and R₃ is a hydrogen atom and the other is —C(O)—(C₁₋₄ alkyl), such as —C(O)—CH₃. In some embodiments, both of R₂ and R₃—C(O)—(C₁₋₄ alkyl), such as —C(O)—CH₃.

In some embodiments, when R₁ is butyl, R₂ and R₅ are not both —C(O)—CH₃.

In some embodiments, the compound of formula (I) is [8]-gingerdioldiacetate, [10]-gingerdioldiacetate, [6]-gingerdiol, [8]-gingerdiol, or [10]-gingerdiol. In some embodiments, the compound of formula (I) is [6]-gingerdioldiacetate, [8]-gingerdioldiacetate,

[10]-gingerdioldiacetate, [6]-gingerdiol, [8]-gingerdiol, or [10]-gingerdiol. In some embodiments, the compound of formula (I) is [8]-gingerdioldiacetate or [10]-gingerdioldiacetate. In some embodiments, the compound of formula (I) is [6]-gingerdiol, [8]-gingerdiol, or [10]-gingerdiol.

Where the compounds disclosed herein have at least one chiral center that is not specifically indicated in the formula, they may exist as individual enantiomers and diastereomers or as mixtures of such isomers. In some embodiments, the sweet-enhancing compound has substantial enantiomeric purity.

Separation of the individual isomers or selective synthesis of the individual isomers is accomplished by application of various methods which are well known to practitioners in the art. Unless otherwise indicated (e.g., where the stereochemistry of a chiral center is explicitly shown), all such isomers and mixtures thereof are included in the scope of the compounds disclosed herein. Furthermore, compounds disclosed herein may exist in one or more crystalline or amorphous forms. Unless otherwise indicated, all such forms are included in the scope of the compounds disclosed herein including any polymorphic forms. In addition, some of the compounds disclosed herein may form solvates with water (i.e., hydrates) or common organic solvents. Unless otherwise indicated, such solvates are included in the scope of the compounds disclosed herein.

The skilled artisan will recognize that some structures described herein may be resonance forms or tautomers of compounds that may be fairly represented by other chemical structures, even when kinetically; the artisan recognizes that such structures may only represent a very small portion of a sample of such compound(s). Such compounds are considered within the scope of the structures depicted, though such resonance forms or tautomers are not represented herein.

Isotopes may be present in the compounds described. Each chemical element as represented in a compound structure may include any isotope of said element. For example, in a compound structure a hydrogen atom may be explicitly disclosed or understood to be present in the compound. At any position of the compound that a hydrogen atom may be present, the hydrogen atom can be any isotope of hydrogen, including but not limited to hydrogen-1 (protium) and hydrogen-2 (deuterium). Thus, reference herein to a compound encompasses all potential isotopic forms unless the context clearly dictates otherwise.

In some embodiments, the compounds disclosed herein are capable of forming acid and/or base salts by virtue of the presence of amino and/or carboxyl groups or groups similar thereto. Physiologically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Physiologically acceptable salts can be formed using inorganic and organic bases. Inorganic bases from which salts can be derived include, for example, bases that contain sodium, potassium, lithium, ammonium, calcium, magnesium, iron, zinc, copper, manganese, aluminum, and the like; particularly preferred are the ammonium, potassium, sodium, calcium and magnesium salts. In some embodiments, treatment of the compounds disclosed herein with an inorganic base results in loss of a labile hydrogen from the compound to afford the salt form including an inorganic cation such as Li⁺, Na⁺, K⁺, Mg²⁺ and Ca²⁺ and the like. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. In some embodiments, the salts are comestibly acceptable salts, which are salts suitable for inclusion in comestible food and/or beverage products.

### Solid State Forms and Solutions of Gingerdiol Compounds

In another aspect, the disclosure provides various solid-state forms of the gingerdiol compounds or their comestibly acceptable salts.

In some embodiments, the gingerdiol compounds or any of their comestibly acceptable salts exists as a crystalline solid, either in substantially pure form or in a formulation such as those set forth below. The crystalline solid can have any suitable polymorphic form, such as any polymorphic form obtainable via recrystallization in any suitable solvent system, according to techniques commonly used in the art of polymorph screening.

In some other embodiments, the gingerdiol compounds or any of their comestibly acceptable salts exists as an amorphous solid or a semi-amorphous solid, meaning that it lacks any regular crystalline structure. Such solids can be generated using standard techniques, such as spray drying, and the like.

In some embodiments, the gingerdiol compounds or any of their comestibly acceptable salts exists as a solvate, which is a pseudomorphic form of the compound in which one or more solvent molecules (such as water molecules) are taken up into the crystalline structure. Any suitable solvent or combination of solvents can be used, including, but not limited to, water, methanol, ethanol, n-propanol, isopropanol, n-butanol, 2-butanol, isobutanol, ethyl acetate, ethylene glycol, 1,2-propylene glycol, 1,3-propylene glycol, and the like. In some embodiments, the disclosure provides hydrates of the conjugates diyne or its comestibly acceptable salts. Such solvates can be generated by any suitable means, such as those techniques typically used by skilled artisans in the field of polymorph and solvate screening.

In some other embodiments, the gingerdiol compounds or any of their comestibly acceptable salts exist as a co-crystal with one or more other compounds, such as one or more other sweetener compounds. The gingerdiol compounds or any of their comestibly acceptable salts can form a co-crystal with any suitable compound. Non-limiting examples of such suitable compounds include fructose, glucose, galactose, sucrose, lactose, maltose, allulose, sugar alcohols (such as erythritol, sorbitol, xylitol, and the like), sucralose, steviol glycosides (such as rebaudioside A, rebaudioside E, rebaudioside M, and the like natural stevioside compounds), mogrosides (such as mogroside V, and other like natural mogroside compounds), aspartame, saccharin, acesulfame K, cyclamate, inulin, isomalt, and maltitol. Such co-crystals can be generated by any suitable means, such as those set forth in U.S. Patent Application Publication No. 2018/0363074, which is incorporated herein by reference.

In some embodiments, the gingerdiol compounds or their comestibly acceptable salts is in the form of a dry particle. Such dry particles can be formed by standard techniques in the art, such as dry granulation, wet granulation, and the like. Such particles can also contain a number of excipients, including, inert diluents, such as calcium carbonate, sodium carbonate, lactose, calcium phosphate, and sodium phosphate; granulating and disintegrating agents, such as starch, cellulosic materials, and alginic acid; binding agents, such as gelatin, guar gum, and acacia; and lubricating agents, such as magnesium stearate, stearic acid, and talc. Other excipients typically used in food and beverage products can also be included, such as typical foodstuff materials.

In some embodiments, the gingerdiol compounds or their comestibly acceptable salts are in the form of a liquid solution or a liquid suspension. Such compositions can also include: carboxymethylcellulose, methylcellulose, hydroxypropylmethylcellulose, sodium alginate, polyvinylpyrrolidone, gum tragacanth and gum acacia; dispersing or wetting agents may be a naturally-occurring phosphatide such as lecithin, or condensation products of an alkylene oxide with fatty acids, for example polyoxyethylene stearate, or condensation products of ethylene oxide with long chain aliphatic alcohols, for example, heptadecaethyl-eneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. Such compositions can also include one or more coloring agents, one or more flavoring agents, and the like. Such liquid suspensions and solutions have a liquid carrier. In general, the liquid carrier comprises water. In some such cases, the liquid composition is an emulsion, such as an oil-in-water or a water-in-oil emulsion. Further, in some cases, water may be too polar to dissolve the gingerdiol compounds to the desired concentration. In such instances, it can be desirable to introduce water-miscible solvents, such as alcohols, glycols, polyols, and the like, to the solvent to enhance solubilization of the gingerdiol compounds.

In some embodiments, the gingerdiol compounds, or their comestibly acceptable salts, is in the form of a solution, i.e., are solvated within a liquid carrier. In some embodiments, the liquid carrier is an aqueous carrier. In some such embodiments, the solutions comprise a comestibly acceptable salt of an gingerdiol compound, such as a hydrochloride salt, a potassium salt, or a sodium salt. Such solutions can be diluted to any suitable concentration.

### Synthetic Processes

In at least one aspect, the disclosure provides a process for making a compound of formula (III):

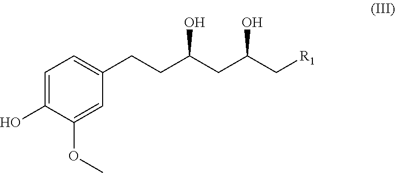

in the form of a mixture or salt thereof; wherein: R₁ is a hydrogen atom or a C₁₋₂₀ alkyl group; and each R₂ and R₃, are, independently from each other, —C(O)—H or —C(O)—(C₁₋₄ alkyl);
the process comprising reducing a precursor compound of formula (II)

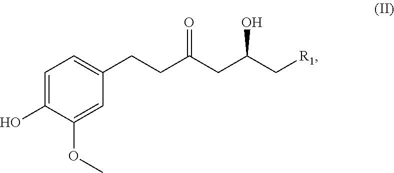

in the form of a mixture or salt thereof, wherein R₁ is defined as in formula (III), with a oxidoreductase enzyme to form a compound of formula (III).

An embodiment of this aspect of the invention is wherein the process further comprises esterifying the compound of formula (III) to form a compound of formula (I),

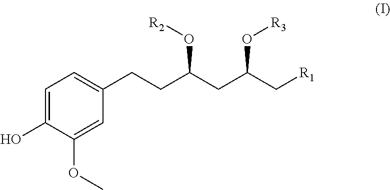

in the form of a mixture or salt thereof; wherein:
R₁ is a hydrogen atom or a C₁₋₂₀ alkyl group; and
each R₂ and R₃, are, independently from each other, —C(O)—H or —C(O)—(C₁₋₄ alkyl);

A further aspect of the invention provides a process for making a compound of formula (I) the process comprising reducing a precursor compound of formula (II) with a oxidoreductase enzyme to form a compound of formula (III), and the esterification of the compound of formula (III) to form a compound of formula (I).

In the preceding embodiments of the present aspect, R₁ can have any suitable value consistent with the definitions set forth above. In some embodiments, R₁ is a C₅₋₁₂ alkyl group. In some embodiments, R₁ is a C₆₋₁₂ alkyl group. In some embodiments, R₁ is butyl. In some embodiments, R₁ is hexyl. In some embodiments, R₁ is octyl. In some embodiments, R₁ is decyl.

In the preceding embodiments of the present aspect, the variables R₂ and R₃ can have any suitable values consistent with the definition set forth above. In some embodiments, both R₂ and R₅ are a hydrogen atom. In some embodiments, one of R₂ and R₃ is a hydrogen atom and the other is —C(O)—(C₁₋₄ alkyl), such as —C(O)—CH₃. In some embodiments, both of R₂ and R₃—C(O)—(C₁₋₄ alkyl), such as —C(O)—CH₃.

In some embodiments, the precursor compound of formula (II) is [6]-gingerol, [8]-gingerol, or [10]-gingerol. Chemical structures for these compounds are shown below.

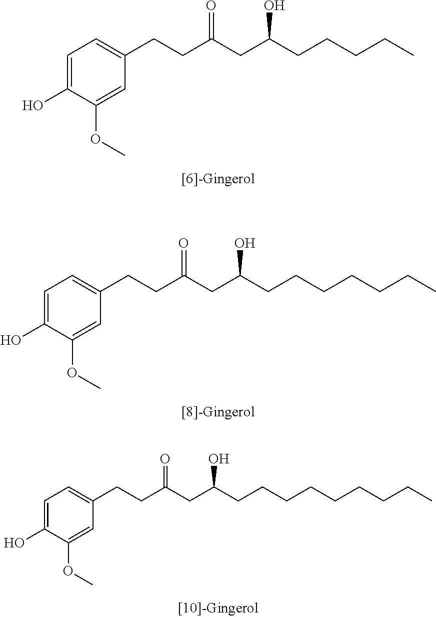 [SMILES: CCCCC[C@H](O)CC(=O)CCc1ccc(O)c(OC)c1 ; CCCCCCC[C@H](O)CC(=O)CCc1ccc(O)c(OC)c1 ; CCCCCCCCC[C@H](O)CC(=O)CCc1ccc(O)c(OC)c1]

The precursor compound of formula (II) can be obtained from synthetic sources or natural products including plants or microbial sources, such as natural plant, fungi, and bacterial sources. Examples of such natural sources include, but are not limited to Aesculus hippocastaneum; Alchemilla xanthochlora; Angelica archangelica; Apocynum cannabinum; Azadirachta indica; Actinomycete bacteria; Capsicum annuum; Cimicifuga racemosa; Commiphora mukul; Embelia ribes; Evodia rutaecarpa; Ferula assa-foetida; Fungi; Gleditschia australis; Kaempferia galanga; Lavandula officinalis; Marrubium vulgare; Mesua ferrea; Nephelium cuspidatum; Orthosiphon stamineus; Persea gratissima; Petroselinum stativum; Piper longum; Pithecoctenium echinatum; Podophyllum peltatum; Psidium guajava; Ricinus communis; Salvia miltiorrhiza; Schisandea chinensis; Teclea trichocarpa; Vitex agnus; Xysmalobium undulatum; Yucca gloriosa; Zanthoxylum piperitum; Zingiber officinalis; and others.

In some embodiments, a plant extract, such as ginger extract, is used to isolate the compounds of formula (II), such as [6]-gingerol, [8]-gingerol, or [10]-gingerol. In some embodiments, diethyl(methoxy)borane and NaBH₄ are used to reduce the precursor compound of formula (II) into the compound of formula (III).

In some embodiments of the invention the compound of formula (III) is esterified into the compound of formula (I).

Various different methods can be used to esterify the compound of formula (III). In some embodiments, the method can use an enzymatic catalysis system. For example, the esterification could be performed using lipases with ethyl acetate, or vinyl acetate as a substrate. Examples of such lipase-based esterification processes are well known in the art and can be used in this embodiment of the invention.

In other embodiments of the invention the compound of formula (III) is esterified into the compound of formula (I) using a chemical reaction. Suitable chemical reagents for esterifying the compound of formula (III) are well known in the art. For example, acetic acid can be used. In such reaction processes, it may be advantageous to provide an acidic catalyst to aid the esterifying reaction. While acids such as sulfuric acid may be used, it is preferred that less corrosive acids are used in the reaction since they are less likely to affect the reaction vessels. For example, PTSA (para toluene sulphonic acid), phosphoric acid, boric acid or hydrofluoric acid can be used.

It is preferred that acetic acid is used to esterify the compound of formula (III) into the compound of formula (I). PTS (para toluene sulphonic acid) or phosphoric acid can be used as an optional catalyst in combination with acetic acid. Preferably the esterification uses acetic acid.

In some embodiments, the compound of formula (III) is [6]-gingerdiol, [8]-gingerdiol, or [10]-gingerdiol. The chemical structures for these compounds are shown below.

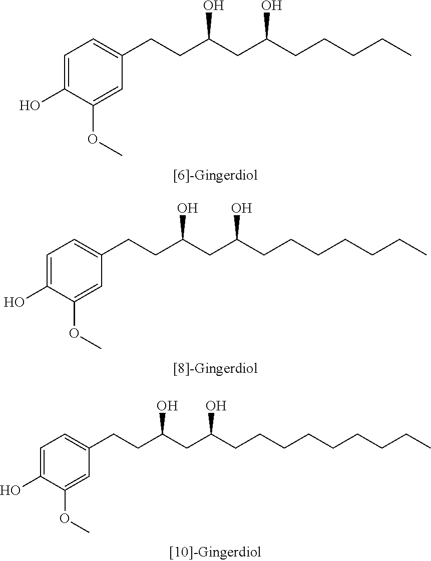 [SMILES: CCCCC[C@H](O)C[C@H](O)CCc1ccc(O)c(OC)c1 ; CCCCCCC[C@H](O)C[C@H](O)CCc1ccc(O)c(OC)c1 ; CCCCCCCCC[C@H](O)C[C@H](O)CCc1ccc(O)c(OC)c1]

In some embodiments, the compound of formula (I) is [6]-gingerdioldiacetate, i.e.,

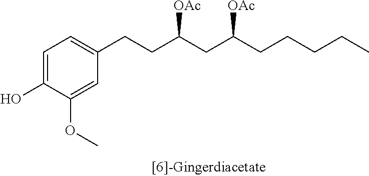 [SMILES: CCCCC[C@@H](C[C@@H](CCc1ccc(O)c(OC)c1)OC(C)=O)OC(C)=O]

In some embodiments, the compound of formula (I) is [8]-gingerdioldiacetate, i.e.,

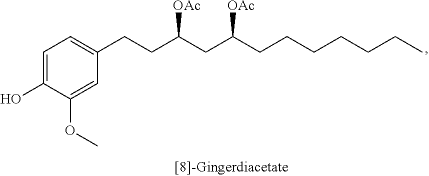 [SMILES: CCCCCCC[C@@H](C[C@@H](CCc1ccc(O)c(OC)c1)OC(C)=O)OC(C)=O]

or [10]-gingerdioldiacetate, i.e.

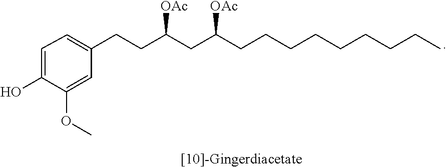 [SMILES: CCCCCCCCC[C@@H](C[C@@H](CCc1ccc(O)c(OC)c1)OC(C)=O)OC(C)=O]

The process for making a compound of formula (III): comprises reducing a precursor compound of formula (II) with a oxidoreductase enzyme to form a compound of formula (III).

An oxidoreductase is an enzyme that catalyzes the transfer of electrons from one molecule, the reductant, also called the electron donor, to another, the oxidant, also called the electron acceptor. A particular group of enzymes like the KRED usually utilizes NAD(P)+ or NAD+ as cofactors. Oxidoreductases comprise the large class of enzymes that catalyze biological oxidation/reduction reactions. Because many chemical and biochemical transformations involve oxidation/reduction processes, oxidoreductases have much utility in the development of biotech methods of synthesis of desirable compounds.

There are several different classes of oxidoreductases which are primarily defined according to their substrate and/or mode of action. For example; ketoreductases, peroxidases, hydroxylases and oxygenases, and reductases.

The present inventors sought to identify whether a oxidoreductase could be used to reduce the compound of formula (II) to form a compound of formula (III). Surprisingly they identified several such enzymes which can be used for this purpose, as shown in the accompanying examples. To the best knowledge of the inventors this is the first time that a oxidoreductase has been used for this reaction.

The oxidoreductases which can be used in the process of the invention are ketoreductases.

Ketoreductase” and “KRED” enzymes are used interchangeably herein to refer to a polypeptide having an enzymatic capability of reducing a carbonyl group to its corresponding alcohol.

Enzymes belonging to the ketoreductase (KRED) (e.g. the broad class EC 1.1.1. and in particular EC 1.1.1.184) are useful for the synthesis of alcohols from the corresponding ketone substrates KREDs typically convert a ketone or aldehyde substrate to the corresponding alcohol product, but may also catalyze the reverse reaction, oxidation (dehydrogenation) of an alcohol substrate to the corresponding ketone/aldehyde product. KRED enzymes can be found in a wide range of bacteria and yeasts.

The reduction of ketones and aldehydes and the oxidation of alcohols by enzymes such as KRED typically involves a co-factor, most commonly reduced nicotinamide adenine dinucleotide (NADH) or reduced nicotinamide adenine dinucleotide phosphate (NADPH), and nicotinamide adenine dinucleotide (NAD) or nicotinamide adenine dinucleotide phosphate (NADP) for the oxidation reaction. NADH and NADPH serve as electron donors, while NAD and NADP serve as electron acceptors. It is frequently observed that ketoreductases and alcohol dehydrogenases accept either the phosphorylated or the non-phosphorylated co-factor (in its oxidized and reduced state).

Examples of such enzymes include a polypeptide encoded the amino acid sequence of SEQ ID Nos: 1 or 3 and the KRED enzyme IEP Ox95 which can be obtained from Cambrex (Cambrex IEP, Wiesbaden, Germany)

Hence a preferred embodiment of the process of the invention is wherein the KRED enzyme has at least 50%, 55%, 60%, 65%, 70%, 75%, 80%, 85%, 90%, 95%, 96%, 97%, 98% or 99% sequence identity to SEQ ID NO: 1 or SEQ ID NO: 3 or comprises the amino acid sequence of SEQ ID NO: 1 or SEQ ID NO: 3, or the KRED enzyme IEP Ox95.

SEQ ID No 1 encodes a KRED polypeptide isolated from Bacillus sp, and SEQ ID No 3 encodes a KRED polypeptide isolated from Weizmannia coagulans. As demonstrated herein they both have enzyme activity which can be used in the processes of the invention. Since the amino acid sequences of and SEQ ID No 1 and 3 share 88% identity, it can be appreciated that it is possible to identify additional KRED enzymes which share sequence identity to SEQ ID No 1 and 3 and retain the ability to perform as KRED enzymes in the processes of the invention.

As is known by those of skill in the art, ketoreductase-catalyzed reduction reactions typically require a cofactor. Reduction reactions catalyzed by the KRED enzymes as described herein also typically require a cofactor. As used herein, the term “cofactor” refers to a non-protein compound that operates in combination with a ketoreductase enzyme. Cofactors suitable for use with the KRED enzymes in the processes of the invention described herein include, but are not limited to, NAD(P)⁺ (nicotinamide adenine dinucleotide phosphate), NAD(P)H (the reduced form of NAD(P)⁺, NAD⁺ (nicotinamide adenine dinucleotide) and NADH (the reduced form of NAD⁺). Generally, the reduced form of the cofactor is added to the reaction mixture.

An embodiment of the process of the invention is wherein the ketoreductase reduction is performed in the presence of a co-factor; preferably the co-factor is NAD(P)H or NAD(P).

The reduced NAD(P)H form can be optionally regenerated from the oxidized NAD(P)⁺ form using a cofactor regeneration system. One benefit of using a co-factor regenerating system is that such a system can push the equilibrium of the process of the invention towards the generation of the desired product, for example [6]-gingerdiol. In this way the process of the invention can be more optimized and efficient in terms of reagents used therefore more timely and cost effective than without the use of a co-factor regenerating system.

Hence an embodiment of the process of the invention is wherein the ketoreductase reduction is performed in the presence of a co-factor regeneration system.

The term “cofactor regeneration system” refers to a set of reactants that participate in a reaction that reduces the oxidized form of the cofactor (e.g., NAD(P)⁺ to NADPH). Cofactors oxidized by the ketoreductase-catalyzed reduction of the keto substrate are regenerated in reduced form by the cofactor regeneration system. Cofactor regeneration systems comprise a stoichiometric reductant that is a source of reducing hydrogen equivalents and is capable of reducing the oxidized form of the cofactor. The cofactor regeneration system may further comprise a catalyst, for example an enzyme catalyst, that catalyzes the reduction of the oxidized form of the cofactor by the reductant. Cofactor regeneration systems to regenerate NADH or NAD(P)H from NAD⁺ or NAD(P)⁺, respectively, are known in the art and may be used in the processes described herein.

The cofactor regeneration system can be in vivo or in vitro. While not wishing to be bound to any specific embodiments, examples of in vivo cofactor regeneration systems include where the cofactor regeneration enzyme that catalyzes the reduction of the oxidized form of the cofactor by the ketoreductase, is synthesized in a cell which also synthesize the ketoreductase enzyme (also termed “whole-cell catalysis). Hence there is a cofactor regeneration within a single cell. In such embodiments the cell is genetically modified to express both the ketoreductase enzyme and the cofactor regeneration enzyme. Examples of polypeptide sequences encoding the ketoreductase enzyme provided herein. Preferably the cofactor regeneration enzyme is a formate dehydrogenase (FDH) or by a glucose dehydrogenase (GDH), and examples of such enzymes and their polypeptide sequences are well known in the art. Wild type organisms such as baker's yeast have been traditionally used for the reduction of carbonyls using e.g. glucose as the co-substrate.

Alternatively the cofactor regeneration system is an in vitro system. In such embodiments, the ketoreductase enzyme and the cofactor regeneration enzyme are prepared as cell free extracts. The method of the invention is then performed in vitro with the enzyme(s) provided to the reaction medium as crude or purified recombinant protein(s), together with the co-factor, and the co-substrate.

An embodiment of the process of the invention is wherein the co-factor regeneration system is a secondary alcohol (preferably isopropanol), a formate dehydrogenase (FDH), or by a glucose dehydrogenase (GDH) system.

In some embodiments, the a co-factor regenerating system may comprise a formate dehydrogenase. The terms “formate dehydrogenase” and “FDH” are used interchangeably herein to refer to an NAD⁺ or NAD(P)⁺-dependent enzyme that catalyzes the conversion of formate and NAD⁺ or NAD(P)⁺ to carbon dioxide and NADH or NAD(P)H, respectively. Formate dehydrogenases that may be suitable for use as cofactor regenerating systems in the ketoreductase-catalyzed reduction reactions described herein include both naturally occurring formate dehydrogenases, as well as non-naturally occurring formate dehydrogenases.

In a preferred embodiment of the process of the invention, the co-factor regeneration system is a formate dehydrogenase (FDH), for example SP22 (Cambrex IEP, Wiesbaden, Germany)

An embodiment of the process of the invention wherein precursor compound of formula (II) is [6]-gingerol, [8]-gingerol, or [10]-gingerol; preferably [6]-gingerol.

An embodiment of the process of the invention wherein compound of formula (III) is—[6]-gingerdiol, [8]-gingerdiol, or [10]-gingerdiol; preferably [6]-gingerdiol.

As mentioned above, the present inventors sought to identify whether a oxidoreductase could be used to reduce the compound of formula (II) to form a compound of formula (III). Several enzymes which can be used for this purpose, as shown in the accompanying examples.

Hence a further aspect of the invention provides an isolated polypeptide having oxidoreductase activity comprising an amino acid sequence having at least 50%, 55%, 60%, 65%, 70%, 75%, 80%, 85%, 90%, 95%, 96%, 97%, 98% or 99% sequence identity to SEQ ID NO: 1 or 3 or comprising the amino acid sequence of SEQ ID NO: 1 or 3.

Further provided herein is an isolated nucleic acid molecule comprising a nucleotide sequence encoding a polypeptide having oxidoreductase activity and comprising an amino acid sequence having at least 50%, 55%, 60%, 65%, 70%, 75%, 80%, 85%, 90%, 95%, 96%, 97%, 98%, or 99% sequence identity to SEQ ID NO: 1 or 3 or comprising the amino acid sequence of SEQ ID NO: 1 or 3.

Further provided is an isolated nucleic acid comprising a nucleotide sequence having at least 75%, 80%, 85%, 90%, 95%, 96%, 97%, 98% or 99% sequence identity to SEQ ID NO: 2 or SEQ ID NO: 4 or comprising the nucleotide sequence of SEQ ID NO: 2 and SEQ ID NO: 4 or the reverse complement thereof

Further provided is an isolated nucleic acid molecule encoding a polypeptide provided herein.

In one aspect provided herein is a vector comprising the nucleic acid molecules described herein. In another aspect, the vector is an expression vector. In a further aspect, the vector is a prokaryotic vector, viral vector or a eukaryotic vector.

Also provided is a non-human host organism or a host cell comprising (1) a nucleic acid molecule described above, or (2) an expression vector comprising said nucleic acid molecule. In one aspect the non-human organism or host cell is a prokaryotic or eukaryotic cell. In another aspect the host cell is a bacterial cell, a plant cell, a fungal cell or a yeast. In a further aspect, the bacterial cell is E. coli and the yeast cell is Saccharomyces cerevisiae

Further provided is the use of a polypeptide described herein for producing a compound of formula (III):

Further provided is a nucleotide sequence obtained by modifying SEQ ID NO: 2 or SEQ ID NO: 4 or the reverse complement thereof which encompasses any sequence that has been obtained by modifying the sequence of SEQ ID NO: 2 or SEQ ID NO: 4, or of the reverse complement thereof using any method known in the art, for example, by introducing any type of mutations such as deletion, insertion and/or substitution mutations. The nucleic acids comprising a sequence obtained by mutation of SEQ ID NO: 2 or SEQ ID NO: 4 or the reverse complement thereof are encompassed by an embodiment herein, provided that the sequences they comprise share at least the defined sequence identity of SEQ ID NO: 2 or SEQ ID NO: 4 or the reverse complement thereof and provided that they encode a polypeptide having oxidoreductase activity, as defined in any of the above embodiments. Mutations may be any kind of mutations of these nucleic acids, for example, point mutations, deletion mutations, insertion mutations and/or frame shift mutations of one or more nucleotides of the DNA sequence of SEQ ID NO: 2 or SEQ ID NO: 4. In one embodiment, the nucleic acid of an embodiment herein may be truncated provided that it encodes a polypeptide as described herein.

A variant nucleic acid may be prepared in order to adapt its nucleotide sequence to a specific expression system. For example, bacterial expression systems are known to more efficiently express polypeptides if amino acids are encoded by particular codons.

Due to the degeneracy of the genetic code, more than one codon may encode the same amino acid sequence, multiple nucleic acid sequences can code for the same protein or polypeptide, all these DNA sequences being encompassed by an embodiment herein. Where appropriate, the nucleic acid sequences encoding the oxidoreductase may be optimized for increased expression in the host cell. For example, nucleotides of an embodiment herein may be synthesized using codons particular to a host for improved expression.

In one embodiment provided herein is an isolated, recombinant or synthetic nucleic acid sequence of SEQ ID NO: 2 or SEQ ID NO: 4 encoding for a polypeptide having oxidoreductase activity comprising the amino acid sequence of SEQ ID NO: 2 or fragments thereof that catalyze production of a compound of formula (III):

Provided herein are also cDNA, genomic DNA and RNA sequences. Any nucleic acid sequence encoding the oxidoreductase or variants thereof is also referred herein as a oxidoreductase encoding sequence.

According to one embodiment, the nucleic acid of SEQ ID NO: 2 or SEQ ID NO: 4 is the coding sequence of an oxidoreductase gene encoding an oxidoreductase obtained as described in the Examples.

A fragment of a polynucleotide of SEQ ID NO: 2 or SEQ ID NO: 4 refers to contiguous nucleotides that is particularly at least 15 bp, at least 30 bp, at least 40 bp, at least 50 bp and/or at least 60 bp in length of the polynucleotide of an embodiment herein. Particularly the fragment of a polynucleotide comprises at least 25, more particularly at least 50, more particularly at least 75, more particularly at least 100, more particularly at least 150, more particularly at least 200, more particularly at least 300, more particularly at least 400, more particularly at least 500, more particularly at least 600, more particularly at least 700, more particularly at least 800, more particularly at least 900, more particularly at least 1000 contiguous nucleotides of the polynucleotide of an embodiment herein. Without being limited, the fragment of the polynucleotides herein may be used as a PCR primer, and/or as a probe, or for anti-sense gene silencing or RNAi.

It is clear to the person skilled in the art that genes, including the polynucleotides of an embodiment herein, can be cloned on basis of the available nucleotide sequence information, such as found in the attached sequence listing, by methods known in the art. These include e.g. the design of DNA primers representing the flanking sequences of such gene of which one is generated in sense orientations and which initiates synthesis of the sense strand and the other is created in reverse complementary fashion and generates the antisense strand. Thermo stable DNA polymerases such as those used in polymerase chain reaction are commonly used to carry out such experiments. Alternatively, DNA sequences representing genes can be chemically synthesized and subsequently introduced in DNA vector molecules that can be multiplied by e.g. compatible bacteria such as e.g. E. coli.

In a related embodiment provided herein, PCR primers and/or probes for detecting nucleic acid sequences encoding a oxidoreductase are provided. The skilled artisan will be aware of methods to synthesize degenerate or specific PCR primer pairs to amplify a nucleic acid sequence encoding the oxidoreductase or fragments thereof, based on SEQ ID NO: 2 or SEQ ID NO: 4. A detection kit for nucleic acid sequences encoding the oxidoreductase may include primers and/or probes specific for nucleic acid sequences encoding the oxidoreductase, and an associated protocol to use the primers and/or probes to detect nucleic acid sequences encoding the oxidoreductase in a sample. Such detection kits may be used to determine whether a plant, organism or cell has been modified, i.e., transformed with a sequence encoding the oxidoreductase.

To test a function of variant DNA sequences according to an embodiment herein, the sequence of interest is operably linked to a selectable or screenable marker gene and expression of the reporter gene is tested in transient expression assays with protoplasts or in stably transformed plants. The skilled artisan will recognize that DNA sequences capable of driving expression are built as modules. Accordingly, expression levels from shorter DNA fragments may be different than the one from the longest fragment and may be different from each other. Provided herein are also functional equivalents of the nucleic acid sequence coding the oxidoreductase proteins provided herein, i.e., nucleotide sequences that hybridize under stringent conditions to the nucleic acid sequence of SEQ ID NO: 2 or SEQ ID NO: 4.

The skilled artisan will be aware of methods to identify homologous sequences in other organisms and methods to determine the percentage of sequence identity between homologous sequences. Such newly identified DNA molecules then can be sequenced and the sequence can be compared with the nucleic acid sequence of SEQ ID NO: 2 or SEQ ID NO: 4.

The percentage of identity between two peptide or nucleotide sequences is a function of the number of amino acids or nucleotide residues that are identical in the two sequences when an alignment of these two sequences has been generated. Identical residues are defined as residues that are the same in the two sequences in a given position of the alignment. The percentage of sequence identity, as used herein, is calculated from the optimal alignment by taking the number of residues identical between two sequences dividing it by the total number of residues in the shortest sequence and multiplying by 100. The optimal alignment is the alignment in which the percentage of identity is the highest possible. Gaps may be introduced into one or both sequences in one or more positions of the alignment to obtain the optimal alignment. These gaps are then taken into account as non-identical residues for the calculation of the percentage of sequence identity. Alignment for the purpose of determining the percentage of amino acid or nucleic acid sequence identity can be achieved in various ways using computer programs and for instance publicly available computer programs available on the world wide web. Preferably, the BLAST program (Tatiana et al, FEMS Microbiol Lett., 1999, 174:247-250, 1999) set to the default parameters, available from the National Center for Biotechnology Information (NCBI) website at ncbi.nlm.nih.gov/BLAST/bl2seq/wblast2.cgi, can be used to obtain an optimal alignment of protein or nucleic acid sequences and to calculate the percentage of sequence identity.

A related embodiment provided herein provides a nucleic acid sequence which is complementary to the nucleic acid sequence according to SEQ ID NO: 2 or SEQ ID NO: 4 such as inhibitory RNAs, or nucleic acid sequence which hybridizes under stringent conditions to at least part of the nucleotide sequence according to SEQ ID NO: 2 or SEQ ID NO: 4. An alternative embodiment of an embodiment herein provides a method to alter gene expression in a host cell. For instance, the polynucleotide of an embodiment herein may be enhanced or overexpressed or induced in certain contexts (e.g. upon exposure to a certain temperature or culture conditions) in a host cell or host organism.

Alteration of expression of a polynucleotide provided herein may also result in ectopic expression which is a different expression pattern in an altered and in a control or wild-type organism. Alteration of expression occurs from interactions of polypeptide of an embodiment herein with exogenous or endogenous modulators, or as a result of chemical modification of the polypeptide. The term also refers to an altered expression pattern of the polynucleotide of an embodiment herein which is altered below the detection level or completely suppressed activity.

In one embodiment, provided herein is also an isolated, recombinant or synthetic polynucleotide encoding a polypeptide or variant polypeptide provided herein.

In one embodiment is provided an isolated nucleic acid molecule encoding a polypeptide having oxidoreductase activity and comprising an amino acid sequence having at least 50%, 55%, 60%, 65%, 70%, 75%, 80%, 85%, 90%, 95%, 96%, 97%, 98%, or 99% sequence identity to SEQ ID NO: 1 or 3 or comprising the amino acid sequence of SEQ ID NO: 1 or 3.

In one embodiment provided herein is an isolated polypeptide having oxidoreductase activity and comprising an amino acid sequence having at least 50%, 55%, 60%, 65%, 70%, 75%, 80%, 85%, 90%, 95%, 96%, 97%, 98%, or 99% sequence identity to SEQ ID NO: 1 or 3 or comprising the amino acid sequence of SEQ ID NO: 1 or 3.

According to one embodiment, the polypeptide consists of the amino acid sequence of SEQ ID NO: 1 or 3.

In one embodiment, the at least one polypeptide having oxidoreductase activity used in any of the herein-described embodiments or encoded by the nucleic acid used in any of the herein-described embodiments comprises an amino acid sequence that is a variant of SEQ ID NO: 1 or 3, obtained by genetic engineering. In one embodiment the polypeptide comprises an amino acid sequence encoded by a nucleotide sequence that has been obtained by modifying SEQ ID NO: 2 or SEQ ID NO: 4 or the reverse complement thereof.

Polypeptides are also meant to include variants and truncated polypeptides provided that they have oxidoreductase activity.

According to another embodiment, the at least one polypeptide having a oxidoreductase activity used in any of the herein-described embodiments or encoded by the nucleic acid used in any of the herein-described embodiments comprises an amino acid sequence that is a variant of SEQ ID NO: 1 or 3, obtained by genetic engineering, provided that said variant has oxidoreductase activity and has the required percentage of identity to SEQ ID NO: 1 or 3 as described herein.

According to another embodiment, the at least one polypeptide having a oxidoreductase activity used in any of the herein-described embodiments or encoded by the nucleic acid used in any of the herein-described embodiments is a variant of SEQ ID NO: 1 or 3 that can be found naturally in other organisms provided that it has a oxidoreductase activity. As used herein, the polypeptide includes a polypeptide or peptide fragment that encompasses the amino acid sequences identified herein, as well as truncated or variant polypeptides provided that they have oxidoreductase activity and that they share at least the defined percentage of identity with the corresponding fragment of SEQ ID NO: 1 or 3.

Examples of variant polypeptides are naturally occurring proteins that result from alternate mRNA splicing events or from proteolytic cleavage of the polypeptides described herein. Variations attributable to proteolysis include, for example, differences in the N- or C-termini upon expression in different types of host cells, due to proteolytic removal of one or more terminal amino acids from the polypeptides of an embodiment herein. Polypeptides encoded by a nucleic acid obtained by natural or artificial mutation of a nucleic acid of an embodiment herein, as described thereafter, are also encompassed by an embodiment herein.

Polypeptide variants resulting from a fusion of additional peptide sequences at the amino and carboxyl terminal ends can also be used in the methods of an embodiment herein. In particular such a fusion can enhance expression of the polypeptides, be useful in the purification of the protein or improve the enzymatic activity of the polypeptide in a desired environment or expression system. Such additional peptide sequences may be signal peptides, for example. Another aspect encompasses methods using variant polypeptides, such as those obtained by fusion with other oligo- or polypeptides and/or those which are linked to signal peptides. Polypeptides resulting from a fusion with another functional protein, can also be advantageously used in the methods of an embodiment herein.

A variant may also differ from the polypeptide of an embodiment herein by attachment of modifying groups which are covalently or non-covalently linked to the polypeptide backbone. The variant also includes a polypeptide which differs from the polypeptide provided herein by introduced N-linked or O-linked glycosylation sites, and/or an addition of cysteine residues. The skilled artisan will recognize how to modify an amino acid sequence and preserve biological activity.

In addition to the gene sequences shown in the sequences disclosed herein, it will be apparent for the person skilled in the art that DNA sequence polymorphisms may exist within a given population, which may lead to changes in the amino acid sequence of the polypeptides disclosed herein. Such genetic polymorphisms may exist in cells from different populations or within a population due to natural allelic variation. Allelic variants may also include functional equivalents.

Further embodiments also relate to the molecules derived by such sequence polymorphisms from the concretely disclosed nucleic acids. These natural variations usually bring about a variance of about 1 to 5% in the nucleotide sequence of a gene or in the amino acid sequence of the polypeptides disclosed herein. As mentioned above, the nucleic acid encoding the polypeptide or variants thereof of an embodiment herein is a useful tool to modify non-human host organisms or cells and to modify non-human host organisms or cells intended to be used in the methods described herein.

An embodiment provided herein provides amino acid sequences of oxidoreductase proteins including orthologs and paralogs as well as methods for identifying and isolating orthologs and paralogs of the oxidoreductase in other organisms. Particularly, so identified orthologs and paralogs of the oxidoreductase and are capable of producing a compound of formula (III).

The oxidoreductase polypeptide can be obtained by extraction from any organism expressing it, using standard protein or enzyme extraction technologies. If the host organism is an unicellular organism or cell releasing the polypeptide of an embodiment herein into the culture medium, the polypeptide may simply be collected from the culture medium, for example by centrifugation, optionally followed by washing steps and re-suspension in suitable buffer solutions. If the organism or cell accumulates the polypeptide within its cells, the polypeptide may be obtained by disruption or lysis of the cells and optionally further extraction of the polypeptide from the cell lysate.

According to another embodiment, the at least one polypeptide having a oxidoreductase can be used in the processes of the invention.

The functionality or activity of any oxidoreductase protein, variant or fragment, may be determined using various methods. For example, transient or stable overexpression in plant, bacterial or yeast cells can be used to test whether the protein has activity, i.e., produces a compound of formula (III). Oxidoreductase activity may be assessed in assay described in the examples herein, indicating functionality. A variant or derivative of a oxidoreductase polypeptide of an embodiment herein retains an ability to produce a compound of formula (III). Amino acid sequence variants of the oxidoreductase provided herein may have additional desirable biological functions including, e.g., altered substrate utilization, reaction kinetics, product distribution or other alterations.

Further provided is at least one vector comprising the nucleic acid molecules described herein.

Also provided herein is a vector selected from the group of a prokaryotic vector, viral vector and a eukaryotic vector.

Further provided here is a vector that is an expression vector.

The nucleic acid sequences of an embodiment herein encoding oxidoreductase proteins can be inserted in expression vectors and/or be contained in chimeric genes inserted in expression vectors, to produce oxidoreductase proteins in a host cell or non-human host organism. The vectors for inserting transgenes into the genome of host cells are well known in the art and include plasmids, viruses, cosmids and artificial chromosomes. Binary or co-integration vectors into which a chimeric gene is inserted can also be used for transforming host cells.

An embodiment provided herein provides recombinant expression vectors comprising a nucleic acid sequence of a oxidoreductase gene, or a chimeric gene comprising a nucleic acid sequence of a oxidoreductase gene, operably linked to associated nucleic acid sequences such as, for instance, promoter sequences. For example, a chimeric gene comprising a nucleic acid sequence of SEQ ID NO: 2 or SEQ ID NO: 4 or a variant thereof may be operably linked to a promoter sequence suitable for expression in plant cells, bacterial cells or fungal cells, optionally linked to a 3′ non-translated nucleic acid sequence.

Alternatively, the promoter sequence may already be present in a vector so that the nucleic acid sequence which is to be transcribed is inserted into the vector downstream of the promoter sequence. Vectors can be engineered to have an origin of replication, a multiple cloning site, and a selectable marker.

In one embodiment, an expression vector comprising a nucleic acid as described herein can be used as a tool for transforming non-human host organisms or host cells suitable to carry out the method of an embodiment herein in vivo.

The expression vectors provided herein may be used in the methods for preparing a genetically transformed non-human host organism and/or host cell, in non-human host organisms and/or host cells harboring the nucleic acids of an embodiment herein and in the methods for making polypeptides having a oxidoreductase activity, as described herein.

Recombinant non-human host organisms and host cells transformed to harbor at least one nucleic acid of an embodiment herein so that it heterologously expresses or over-expresses at least one polypeptide of an embodiment herein are also very useful tools to carry out the method of an embodiment herein. Such non-human host organisms and host cells are therefore provided herein.

In one embodiment is provided a host cell or non-human host organism comprising at least one of the nucleic acid molecules described herein or comprising at least one vector comprising at least one of the nucleic acid molecules.

A nucleic acid according to any of the above-described embodiments can be used to transform the non-human host organisms and cells and the expressed polypeptide can be any of the above-described polypeptides.

In one embodiment, the non-human host organism or host cell is a prokaryotic cell. In another embodiment, the non-human host organism or host cell is a bacterial cell. In a further embodiment, the non-human host organism or host cell is Escherichia coli.

In one embodiment, the non-human host organism or host cell is a eukaryotic cell. In another embodiment, the non-human host organism or host cell is a yeast cell. In a further embodiment, the non-human host organism or cell is Saccharomyces cerevisiae.

In one embodiment the non-human host organism or host cell expresses a polypeptide, provided that the organism or cell is transformed to harbor a nucleic acid encoding said polypeptide, this nucleic acid is transcribed to mRNA and the polypeptide is found in the host organism or cell.

Suitable methods to transform a non-human host organism or a host cell have been previously described and are also provided herein.

To carry out an embodiment herein in vivo, the host organism or host cell is cultivated under conditions conducive to the production of a compound of formula (III). If the host is a unicellular organism, conditions conducive to the production of a compound of formula (III) may comprise addition of suitable cofactors to the culture medium of the host. In addition, a culture medium may be selected, so as to maximize a compound of formula (III) synthesis. Examples of optimal culture conditions are described in a more detailed manner in the examples.

Non-human host organisms suitable to carry out the method of an embodiment herein in vivo may be any non-human multicellular or unicellular organisms. In one embodiment, the non-human host organism used to carry out an embodiment herein in vivo is a plant, a prokaryote or a fungus. Any plant, prokaryote or fungus can be used. In another embodiment the non-human host organism used to carry out the method of an embodiment herein in vivo is a microorganism. Any microorganism can be used, for example, the microorganism can be a bacteria or yeast, such as E. coli or Saccharomyces cerevisiae.

Isolated higher eukaryotic cells can also be used, instead of complete organisms, as hosts to carry out the method of an embodiment herein in vivo. Suitable eukaryotic cells may be any non-human cell, such as plant or fungal cells.

Further provided here is a method comprising transforming a host cell or a non-human host organism with a nucleic acid encoding a polypeptide having oxidoreductase activity and comprising an amino acid sequence having at least 50%, 55%, 60%, 65%, 70%, 75%, 80%, 85%, 90%, 95%, 96%, 97%, 98%, or 99% sequence identity to SEQ ID NO: 1 or 3 or comprising the amino acid sequence of SEQ ID NO: 1 or 3.

In one embodiment, a method provided herein comprises cultivating a non-human host organism or a host cell transformed to express a polypeptide wherein the polypeptide comprises a sequence of amino acids that has at least 75%, 80%, 85%, 90%, 95%, 98%, 99% or 100% sequence identity to SEQ ID NO: 1 or SEQ ID NO: 3 under conditions that allow for the production of the polypeptide.

The following examples are illustrative only and are not intended to limit the scope of the claims an embodiments described herein.

### Uses, Methods, and Formulations

In other aspects, the disclosure provides formulations, uses, and methods of using the gingerdiol compounds or their comestibly acceptable salts (in any form according to the preceding aspects and embodiments thereof).

In certain aspects, the disclosure provides uses of any gingerdiol compounds of the preceding aspects or embodiments.

In certain aspects, the disclosure provides uses of any gingerdiol compounds of the preceding aspects or embodiments to modify a flavor of an ingestible composition. In some embodiments, the ingestible composition is a flavored product such as a flavored food or beverage product, or an oral care product.

In certain aspects, the disclosure provides uses of any gingerdiol compounds of the preceding aspects or embodiments to enhance a salty taste of an ingestible composition. In related aspects, the disclosure provides uses of any gingerdiol compounds of the preceding aspects or embodiments and to reduce the salt (e.g., sodium chloride) content of an ingestible composition. In some embodiments of these aspects, the ingestible composition comprises sodium chloride. In some embodiments, the ingestible composition is a flavored product such as a flavored food or beverage product.

In certain aspects, the disclosure provides uses of any gingerdiol compounds of the preceding aspects or embodiments to enhance an umami taste of an ingestible composition. In related aspects, the disclosure provides uses of any gingerdiol compounds of the preceding aspects or embodiments to reduce or eliminate the glutamate or aspartate content of an ingestible composition. In some embodiments of these aspects, the ingestible composition is substantially free of monosodium glutamate (MSG). In some embodiments, the ingestible composition is a flavored product such as a flavored food or beverage product.

In certain aspects, the disclosure provides uses of any gingerdiol compounds of the preceding aspects or embodiments, to enhance a warming or heating effect of an ingestible composition. In some embodiments, the ingestible composition is a flavored product such as a flavored food or beverage product.

In certain aspects, the disclosure provides uses of any gingerdiol compounds of the preceding aspects or embodiments, to enhance a cooling effect of an ingestible composition. In some embodiments of these aspects, the ingestible composition comprises sodium chloride. In some embodiments, the ingestible composition is a flavored product such as a flavored food or beverage product. In some embodiments, the ingestible composition is an oral care product, such as a mouthwash, a toothpaste, a whitening agent, a dentifrice, and the like. In some embodiments, the ingestible composition comprises menthol.

In certain aspects, the disclosure provides uses of any compounds of the first or second aspects to enhance a sweet taste of an ingestible composition. In related aspects, the disclosure provides uses of any gingerdiol compounds of the preceding aspects or embodiments to reduce or eliminate the sweetener (e.g., sucrose, fructose, sucralose, etc.) content of an ingestible composition. In some embodiments of these aspects, the ingestible composition is substantially free of caloric sweetener. In some embodiments, the ingestible composition is a flavored product such as a flavored food or beverage product.

In certain aspects, the disclosure provides uses of any gingerdiol compounds of the preceding aspects or embodiments to reduce the sourness of an ingestible composition.

In certain aspects, the disclosure provides uses of any gingerdiol compounds of the preceding aspects or embodiments to reduce the bitterness of an ingestible composition.

In certain aspects, the disclosure provides methods of modifying the flavor of an ingestible composition, comprising introducing any gingerdiol compounds of the preceding aspects or embodiments to an ingestible composition. In some embodiments, the ingestible composition is a food or beverage product.

The disclosure also provides methods that correspond to certain of the uses set forth in the preceding paragraphs.

In certain aspects, the disclosure provides methods of modifying the flavor of an ingestible composition, comprising introducing any gingerdiol compounds of the preceding aspects or embodiments to an ingestible composition. In some embodiments, the ingestible composition is a food or beverage product.

In certain aspects, the disclosure provides methods of enhancing a salty taste of an ingestible composition, comprising introducing any gingerdiol compounds of the preceding aspects or embodiments to the ingestible composition. In a related aspect, the disclosure provides methods of reducing salt (e.g., sodium chloride) content of an ingestible composition, the method comprising introducing any gingerdiol compounds of the preceding aspects or embodiments to the ingestible composition. In some embodiments, the ingestible composition is a food or beverage product.

In certain aspects, the disclosure provides methods of enhancing an umami taste of an ingestible composition, comprising introducing any gingerdiol compounds of the preceding aspects or embodiments to the ingestible composition. In a related aspect, the disclosure provides methods of reducing or eliminating glutamate (e.g., monosodium glutamate) or aspartate content of an ingestible composition, the method comprising introducing any gingerdiol compounds of the preceding aspects or embodiments to the ingestible composition. In some embodiments, the ingestible composition is a food or beverage product.

In certain aspects, the disclosure provides methods of enhancing a kokumi taste of an ingestible composition, comprising introducing any gingerdiol compounds of the preceding aspects or embodiments to the ingestible composition. In a related aspect, the disclosure provides methods of reducing or eliminating glutamyl (e.g., L-glutamyl peptides) content of an ingestible composition, the method comprising introducing any compounds of the first or second aspects to the ingestible composition. In another related aspect, the disclosure provides methods of reducing or eliminating animal (e.g., animal broth or meat) content of an ingestible composition, the method comprising introducing any gingerdiol compounds of the preceding aspects or embodiments to the ingestible composition. In some embodiments, the ingestible composition is a food or beverage product.

In certain aspects, the disclosure provides methods of enhancing a warming or heating effect of an ingestible composition, comprising introducing any gingerdiol compounds of the preceding aspects or embodiments to the ingestible composition. In some embodiments, the ingestible composition is a food or beverage product.

In certain aspects, the disclosure provides methods of enhancing a cooling effect of an ingestible composition, comprising introducing any gingerdiol compounds of the preceding aspects or embodiments to the ingestible composition. In some embodiments, the ingestible composition is a food or beverage product. In some embodiments, the ingestible composition is an oral care product, such as a mouthwash, a toothpaste, a whitening agent, a dentifrice, and the like. In some embodiments, the ingestible composition comprises menthol.

In certain aspects, the disclosure provides methods of enhancing a sweet taste of an ingestible composition, comprising introducing any gingerdiol compounds of the preceding aspects or embodiments to the ingestible composition. In a related aspect, the disclosure provides methods of reducing or eliminating sweetener (e.g., sucrose, fructose, sucralose, etc.) content of an ingestible composition, the method comprising introducing any gingerdiol compounds of the preceding aspects or embodiments to the ingestible composition. In some embodiments, the ingestible composition is a food or beverage product.

In certain aspects, the disclosure provides methods of reducing a sour taste of an ingestible composition, comprising introducing any gingerdiol compounds of the preceding aspects or embodiments to the ingestible composition. In some embodiments, the ingestible composition is a food or beverage product.

In certain aspects, the disclosure provides methods of reducing a bitter taste of an ingestible composition, comprising introducing any gingerdiol compounds of the preceding aspects or embodiments to the ingestible composition. In some embodiments, the ingestible composition is a food or beverage product.

The foregoing uses and methods generally involve the use or introduction of the gingerdiol compounds to an ingestible composition having one or more additional components or ingredients. For example, in at least one aspect, the disclosure provides compositions comprising any gingerdiol compounds of the foregoing aspects.

In certain particular embodiments, the ingestible composition comprises monosodium glutamate and an gingerdiol compound (or comestibly acceptable salts thereof). In some such embodiments, the introduction of the gingerdiol compound (or comestibly acceptable salt thereof) permits the use of less monosodium glutamate (such as more than 10% less, more than 20% less, more than 30% less, more than 40% less, more than 50% less, more than 60% less, or more than 70% less, or more than 80% less, or more than 90% less) and still achieve a level of umami taste of a comparable product that employs a higher concentration of monosodium glutamate. In some related embodiments, the use of the gingerdiol compounds, or its comestibly acceptable salts, permits the elimination of monosodium glutamate from the composition. In some embodiments, the concentration of the gingerdiol compound, or its comestibly acceptable salts, is no more than 1000 ppm, or no more than 900 ppm, or no more than 800 ppm, or no more than 700 ppm, or no more than 600 ppm, or no more than 500 ppm, or no more than 400 ppm, or no more than 300 ppm, or no more than 200 ppm, or no more than 100 ppm, or no more than 50 ppm, or no more than 25 ppm, or no more than 10 ppm. Such ingestible compositions can be in any suitable form. In some embodiments, the ingestible composition is a food product, such as any of those specifically listed below. In other embodiments, the ingestible composition is a beverage product, such as a soda, and the like.

In certain particular embodiments, the ingestible composition comprises fat, such as animal or vegetable fat, and the gingerdiol compound (or comestibly acceptable salts thereof). In some such embodiments, the introduction of the gingerdiol compound (or comestibly acceptable salt thereof) permits one to use less fat (such as more than 10% less, more than 20% less, more than 30% less, more than 40% less, more than 50% less, more than 60% less, or more than 70% less, or more than 80% less, or more than 90% less) and still achieve a level of umami characteristic of a comparable product that employs a higher concentration of fat. In some related embodiments, the use of the gingerdiol compounds, or its comestibly acceptable salts, permits the elimination of fat from the composition. In some embodiments, the concentration of the gingerdiol compounds, or its comestibly acceptable salts, is no more than 1000 ppm, or no more than 900 ppm, or no more than 800 ppm, or no more than 700 ppm, or no more than 600 ppm, or no more than 500 ppm, or no more than 400 ppm, or no more than 300 ppm, or no more than 200 ppm, or no more than 100 ppm, or no more than 50 ppm, or no more than 25 ppm, or no more than 10 ppm. Such ingestible compositions can be in any suitable form. In some embodiments, the ingestible composition is a food product, such as any of those specifically listed below. In other embodiments, the ingestible composition is a beverage product, such as a soda, and the like. The fat can be any suitable fat, such as a fat derived from an animal or vegetable fat, including, but not limited to, milk fat (including fat in various cheeses), beef fat, pork fat, poultry fat, lamb fat, goat fat, fish oil, olive oil, canola oil, corn oil, safflower oil, nut oil, peanut oil, cashew oil, soybean oil, palm oil, palm kernel oil, coconut oil, butter, and nut butters (such as peanut butter, cashew butter, almond butter, hazelnut butter, and the like).

In certain particular embodiments, the ingestible composition comprises glutamate (including in its free acid form), and the gingerdiol compound (or comestibly acceptable salts thereof). In some such embodiments, the introduction of the gingerdiol compound (or comestibly acceptable salt thereof) permits one to use less glutamate (such as more than 10% less, more than 20% less, more than 30% less, more than 40% less, more than 50% less, more than 60% less, or more than 70% less, or more than 80% less, or more than 90% less) and still achieve a level of umami characteristic of a comparable product that employs a higher concentration of glutamate. In some related embodiments, the use of the gingerdiol compound, or its comestibly acceptable salts, permits the elimination of glutamate from the composition. In some embodiments, the concentration of the gingerdiol compound, or its comestibly acceptable salts, is no more than 1000 ppm, or no more than 900 ppm, or no more than 800 ppm, or no more than 700 ppm, or no more than 600 ppm, or no more than 500 ppm, or no more than 400 ppm, or no more than 300 ppm, or no more than 200 ppm, or no more than 100 ppm, or no more than 50 ppm, or no more than 25 ppm, or no more than 10 ppm. Such ingestible compositions can be in any suitable form. In some embodiments, the ingestible composition is a food product, such as any of those specifically listed below. In other embodiments, the ingestible composition is a beverage product, such as a soda, and the like. The glutamate can be from any suitable source, such as monosodium glutamate, proteins containing glutamic acid (e.g., glutathione), and the like.

In certain particular embodiments, the ingestible composition comprises aspartate (including in its free acid form), and the gingerdiol compound (or comestibly acceptable salts thereof). In some such embodiments, the introduction of the gingerdiol compound (or comestibly acceptable salt thereof) permits one to use less aspartate (such as more than 10% less, more than 20% less, more than 30% less, more than 40% less, more than 50% less, more than 60% less, or more than 70% less, or more than 80% less, or more than 90% less) and still achieve a level of umami characteristic of a comparable product that employs a higher concentration of aspartate. In some related embodiments, the use of the gingerdiol compound, or its comestibly acceptable salts, permits the elimination of aspartate from the composition. In some embodiments, the concentration of the gingerdiol compound, or its comestibly acceptable salts, is no more than 1000 ppm, or no more than 900 ppm, or no more than 800 ppm, or no more than 700 ppm, or no more than 600 ppm, or no more than 500 ppm, or no more than 400 ppm, or no more than 300 ppm, or no more than 200 ppm, or no more than 100 ppm, or no more than 50 ppm, or no more than 25 ppm, or no more than 10 ppm. Such ingestible compositions can be in any suitable form. In some embodiments, the ingestible composition is a food product, such as any of those specifically listed below. In other embodiments, the ingestible composition is a beverage product, such as a soda, and the like. The aspartate can be from any suitable source, such as proteins containing aspartic acid, and the like.

In certain particular embodiments, the ingestible composition comprises animal products, and the gingerdiol compound (or comestibly acceptable salts thereof). In some such embodiments, the introduction of the gingerdiol compound (or comestibly acceptable salt thereof) permits one to use less animal products (such as more than 10% less, more than 20% less, more than 30% less, more than 40% less, more than 50% less, more than 60% less, or more than 70% less, or more than 80% less, or more than 90% less) and still achieve a level of umami characteristic of a comparable product that employs a higher concentration of animal products. In some related embodiments, the use of the gingerdiol compound, or its comestibly acceptable salts, permits the elimination of animal products from the composition. In some embodiments, the concentration of the gingerdiol compound, or its comestibly acceptable salts, is no more than 1000 ppm, or no more than 900 ppm, or no more than 800 ppm, or no more than 700 ppm, or no more than 600 ppm, or no more than 500 ppm, or no more than 400 ppm, or no more than 300 ppm, or no more than 200 ppm, or no more than 100 ppm, or no more than 50 ppm, or no more than 25 ppm, or no more than 10 ppm. Such ingestible compositions can be in any suitable form. In some embodiments, the ingestible composition is a food product, such as any of those specifically listed below. In other embodiments, the ingestible composition is a beverage product, such as a soda, and the like. The animal products can be any suitable animal product, such as cheese, milk, meat broth (such as beef broth, pork broth, chicken broth, turkey broth, duck broth, lamb broth, goat broth, rabbit broth, and the like), eggs, bone broth, bone marrow, meat (such as beef, pork, chicken, lamb, goat, turkey, duck, rabbit, and the like), butter, and animal skin.

In certain particular embodiments, the ingestible composition comprises vegetable products, and the gingerdiol compounds (or comestibly acceptable salts thereof). In some such embodiments, the introduction of the gingerdiol compound (or comestibly acceptable salt thereof) permits one to use less vegetable product (such as more than 10% less, more than 20% less, more than 30% less, more than 40% less, more than 50% less, more than 60% less, or more than 70% less, or more than 80% less, or more than 90% less) and still achieve a level of umami characteristic of a comparable product that employs a higher concentration of vegetable products. In some related embodiments, the use of the gingerdiol compound, or its comestibly acceptable salts, permits the elimination of vegetable products from the composition. In some embodiments, the concentration of the gingerdiol compound, or its comestibly acceptable salts, is no more than 1000 ppm, or no more than 900 ppm, or no more than 800 ppm, or no more than 700 ppm, or no more than 600 ppm, or no more than 500 ppm, or no more than 400 ppm, or no more than 300 ppm, or no more than 200 ppm, or no more than 100 ppm, or no more than 50 ppm, or no more than 25 ppm, or no more than 10 ppm. Such ingestible compositions can be in any suitable form. In some embodiments, the ingestible composition is a food product, such as any of those specifically listed below. In other embodiments, the ingestible composition is a beverage product, such as a soda, and the like. The vegetable products can be any suitable vegetable product, such as celery, celeriac, tomato, garlic, onion, leek, scallion, spices, and the like.

In certain particular embodiments, the ingestible composition comprises sodium (i.e., sodium cation), and the gingerdiol compound (or comestibly acceptable salts thereof). In some such embodiments, the introduction of the gingerdiol compound (or comestibly acceptable salt thereof) permits one to use less sodium (such as more than 10% less, more than 20% less, more than 30% less, more than 40% less, more than 50% less, more than 60% less, or more than 70% less, or more than 80% less, or more than 90% less) and still achieve a level of salty characteristic of a comparable product that employs a higher concentration of sodium. In some related embodiments, the use of the gingerdiol compound, or its comestibly acceptable salts, permits the elimination of sodium from the composition. In some embodiments, the concentration of the gingerdiol compound, or its comestibly acceptable salts, is no more than 1000 ppm, or no more than 900 ppm, or no more than 800 ppm, or no more than 700 ppm, or no more than 600 ppm, or no more than 500 ppm, or no more than 400 ppm, or no more than 300 ppm, or no more than 200 ppm, or no more than 100 ppm, or no more than 50 ppm, or no more than 25 ppm, or no more than 10 ppm. Such ingestible compositions can be in any suitable form. In some embodiments, the ingestible composition is a food product, such as any of those specifically listed below. In other embodiments, the ingestible composition is a beverage product, such as a soda, and the like. The sodium can be any suitable animal product, such as table salt (sodium chloride), sea salt, soy sauce, fish sauce, shrimp paste, butter, miso, and Worcestershire sauce.

In certain particular embodiments, the ingestible composition comprises alcohol, and the gingerdiol compound (or comestibly acceptable salts thereof). In some such embodiments, the introduction of the gingerdiol compound (or comestibly acceptable salt thereof) permits one to use less alcohol (such as more than 10% less, more than 20% less, more than 30% less, more than 40% less, more than 50% less, more than 60% less, or more than 70% less, or more than 80% less, or more than 90% less) and still achieve a level of umami and/or kokumi characteristic of a comparable product that employs a higher concentration of alcohol. In some related embodiments, the use of the gingerdiol compound, or its comestibly acceptable salts, permits the elimination of alcohol from the composition. In some embodiments, the concentration of the gingerdiol compound, or its comestibly acceptable salts, is no more than 1000 ppm, or no more than 900 ppm, or no more than 800 ppm, or no more than 700 ppm, or no more than 600 ppm, or no more than 500 ppm, or no more than 400 ppm, or no more than 300 ppm, or no more than 200 ppm, or no more than 100 ppm, or no more than 50 ppm, or no more than 25 ppm, or no more than 10 ppm. Such ingestible compositions can be in any suitable form. In some embodiments, the ingestible composition is a food product, such as any of those specifically listed below. In other embodiments, the ingestible composition is a beverage product, such as a soda (such as a hard soda), and the like. The alcohol can present in any suitable form, such as alcohol formed from grains, cane sugar, fruits, and the like.

In some instances, one may be able to reduce the amount of sweetener in a product by enhancing the umami or kokumi taste.

In certain particular embodiments, the ingestible composition comprises sucrose and the gingerdiol compound or any of its comestibly acceptable salts. In some such embodiments, the introduction of the gingerdiol compound (or salt) permits one to use less sucrose (such as more than 10% less, more than 20% less, more than 30% less, more than 40% less, more than 50% less, more than 60% less, or more than 70% less) and still achieve a level of sweetness, umami, and/or kokumi characteristic of a comparable product that employs more sucrose. In some embodiments, the concentration of the gingerdiol compound, or its comestibly acceptable salts, is no more than 1000 ppm, or no more than 900 ppm, or no more than 800 ppm, or no more than 700 ppm, or no more than 600 ppm, or no more than 500 ppm, or no more than 400 ppm, or no more than 300 ppm, or no more than 200 ppm, or no more than 100 ppm, or no more than 50 ppm, or no more than 25 ppm, or no more than 10 ppm. In some embodiments, the ingestible composition is a food product, such as any of those specifically listed below. In other embodiments, the ingestible composition is a beverage product, such as a soda, and the like.

In certain particular embodiments, the ingestible composition comprises fructose and the gingerdiol compound or any of its comestibly acceptable salts. In some such embodiments, the introduction of the gingerdiol compound (or salt) permits one to use less fructose (such as more than 10% less, more than 20% less, more than 30% less, more than 40% less, more than 50% less, more than 60% less, or more than 70% less) and still achieve a level of sweetness, umami, and/or kokumi characteristic of a comparable product that employs more fructose. In some embodiments, the concentration of the gingerdiol compound, or its comestibly acceptable salts, is no more than 1000 ppm, or no more than 900 ppm, or no more than 800 ppm, or no more than 700 ppm, or no more than 600 ppm, or no more than 500 ppm, or no more than 400 ppm, or no more than 300 ppm, or no more than 200 ppm, or no more than 100 ppm, or no more than 50 ppm, or no more than 25 ppm, or no more than 10 ppm. In some embodiments, the ingestible composition is a food product, such as any of those specifically listed below. In other embodiments, the ingestible composition is a beverage product, such as a soda, and the like.

In certain particular embodiments, the ingestible composition comprises high-fructose corn syrup and the gingerdiol compound or any of its comestibly acceptable salts. In some such embodiments, the introduction of the gingerdiol compound (or salt) permits one to use less high-fructose corn syrup (such as more than 10% less, more than 20% less, more than 30% less, more than 40% less, more than 50% less, more than 60% less, or more than 70% less) and still achieve a level of sweetness, umami, and/or kokumi characteristic of a comparable product that employs more high-fructose corn syrup. In some embodiments, the concentration of the gingerdiol compound, or its comestibly acceptable salts, is no more than 1000 ppm, or no more than 900 ppm, or no more than 800 ppm, or no more than 700 ppm, or no more than 600 ppm, or no more than 500 ppm, or no more than 400 ppm, or no more than 300 ppm, or no more than 200 ppm, or no more than 100 ppm, or no more than 50 ppm, or no more than 25 ppm, or no more than 10 ppm. In some embodiments, the ingestible composition is a food product, such as any of those specifically listed below. In other embodiments, the ingestible composition is a beverage product, such as a soda, and the like.

In certain particular embodiments, the ingestible composition comprises glucose (for example, D-glucose, in either its alpha or beta forms, or a combination thereof) and the gingerdiol compound or any of its comestibly acceptable salts. In some such embodiments, the introduction of the gingerdiol compound (or salt) permits one to use less glucose (such as more than 10% less, more than 20% less, more than 30% less, more than 40% less, more than 50% less, more than 60% less, or more than 70% less) and still achieve a level of sweetness, umami, and/or kokumi characteristic of a comparable product that employs more glucose. In some embodiments, the concentration of the gingerdiol compound, or its comestibly acceptable salts, is no more than 1000 ppm, or no more than 900 ppm, or no more than 800 ppm, or no more than 700 ppm, or no more than 600 ppm, or no more than 500 ppm, or no more than 400 ppm, or no more than 300 ppm, or no more than 200 ppm, or no more than 100 ppm, or no more than 50 ppm, or no more than 25 ppm, or no more than 10 ppm. Such ingestible compositions can be in any suitable form. In some embodiments, the ingestible composition is a food product, such as any of those specifically listed below. In other embodiments, the ingestible composition is a beverage product, such as a soda, and the like. The glucose can be introduced in any suitable form, such as natural syrups and the like.

In certain particular embodiments, the ingestible composition comprises sucralose and the gingerdiol compound or any of its comestibly acceptable salts. In some such embodiments, the introduction of the gingerdiol compound (or salt) permits one to use less sucralose (such as more than 10% less, more than 20% less, more than 30% less, more than 40% less, more than 50% less, more than 60% less, or more than 70% less) and still achieve a level of sweetness, umami, and/or kokumi characteristic of a comparable product that employs more sucralose. In some embodiments, the concentration of the gingerdiol compound, or its comestibly acceptable salts, is no more than 1000 ppm, or no more than 900 ppm, or no more than 800 ppm, or no more than 700 ppm, or no more than 600 ppm, or no more than 500 ppm, or no more than 400 ppm, or no more than 300 ppm, or no more than 200 ppm, or no more than 100 ppm, or no more than 50 ppm, or no more than 25 ppm, or no more than 10 ppm.

Such ingestible compositions can be in any suitable form. In some embodiments, the ingestible composition is a food product, such as any of those specifically listed below. In other embodiments, the ingestible composition is a beverage product, such as a soda, and the like.

In certain particular embodiments, the ingestible composition comprises rebaudiosides (such as rebaudioside A, rebaudioside D, rebaudioside E, rebaudioside M, or any combination thereof) and the gingerdiol compound or any of its comestibly acceptable salts. In some such embodiments, the introduction of the gingerdiol compound (or salt) permits one to use less rebaudioside (such as more than 10% less, more than 20% less, more than 30% less, more than 40% less, more than 50% less, more than 60% less, or more than 70% less) and still achieve a level of sweetness, umami, and/or kokumi characteristic of a comparable product that employs more rebaudioside. In some embodiments, the concentration of the gingerdiol compound, or its comestibly acceptable salts, is no more than 1000 ppm, or no more than 900 ppm, or no more than 800 ppm, or no more than 700 ppm, or no more than 600 ppm, or no more than 500 ppm, or no more than 400 ppm, or no more than 300 ppm, or no more than 200 ppm, or no more than 100 ppm, or no more than 50 ppm, or no more than 25 ppm, or no more than 10 ppm. Such ingestible compositions can be in any suitable form. In some embodiments, the ingestible composition is a food product, such as any of those specifically listed below. In other embodiments, the ingestible composition is a beverage product, such as a soda, and the like.

In certain particular embodiments, the ingestible composition comprises acefulfame K and the gingerdiol compound or any of its comestibly acceptable salts. In some such embodiments, the introduction of the gingerdiol compound (or salt) permits one to use less acesulfame K (such as more than 10% less, more than 20% less, more than 30% less, more than 40% less, more than 50% less, more than 60% less, or more than 70% less) and still achieve a level of sweetness, umami, and/or kokumi characteristic of a comparable product that employs more acesulfame K. In some embodiments, the concentration of the gingerdiol compound, or its comestibly acceptable salts, is no more than 1000 ppm, or no more than 900 ppm, or no more than 800 ppm, or no more than 700 ppm, or no more than 600 ppm, or no more than 500 ppm, or no more than 400 ppm, or no more than 300 ppm, or no more than 200 ppm, or no more than 100 ppm, or no more than 50 ppm, or no more than 25 ppm, or no more than 10 ppm. Such ingestible compositions can be in any suitable form. In some embodiments, the ingestible composition is a food product, such as any of those specifically listed below. In other embodiments, the ingestible composition is a beverage product, such as a soda, and the like.

In certain particular embodiments, the ingestible composition comprises allulose and the gingerdiol compound or any of its comestibly acceptable salts. In some such embodiments, the introduction of the gingerdiol compound (or salt) permits one to use less allulose (such as more than 10% less, more than 20% less, more than 30% less, more than 40% less, more than 50% less, more than 60% less, or more than 70% less) and still achieve a level of sweetness, umami, and/or kokumi characteristic of a comparable product that employs more allulose. In some embodiments, the concentration of the gingerdiol compound, or its comestibly acceptable salts, is no more than 1000 ppm, or no more than 900 ppm, or no more than 800 ppm, or no more than 700 ppm, or no more than 600 ppm, or no more than 500 ppm, or no more than 400 ppm, or no more than 300 ppm, or no more than 200 ppm, or no more than 100 ppm, or no more than 50 ppm, or no more than 25 ppm, or no more than 10 ppm. Such ingestible compositions can be in any suitable form. In some embodiments, the ingestible composition is a food product, such as any of those specifically listed below. In other embodiments, the ingestible composition is a beverage product, such as a soda, and the like.

In certain particular embodiments, the ingestible composition comprises erythritol and the gingerdiol compound or any of its comestibly acceptable salts. In some such embodiments, the introduction of the gingerdiol compound (or salt) permits one to use less erythritol (such as more than 10% less, more than 20% less, more than 30% less, more than 40% less, more than 50% less, more than 60% less, or more than 70% less) and still achieve a level of sweetness, umami, and/or kokumi characteristic of a comparable product that employs more erythritol. In some embodiments, the concentration of the gingerdiol compound, or its comestibly acceptable salts, is no more than 1000 ppm, or no more than 900 ppm, or no more than 800 ppm, or no more than 700 ppm, or no more than 600 ppm, or no more than 500 ppm, or no more than 400 ppm, or no more than 300 ppm, or no more than 200 ppm, or no more than 100 ppm, or no more than 50 ppm, or no more than 25 ppm, or no more than 10 ppm. Such ingestible compositions can be in any suitable form. In some embodiments, the ingestible composition is a food product, such as any of those specifically listed below. In other embodiments, the ingestible composition is a beverage product, such as a soda, and the like.

In certain particular embodiments, the ingestible composition comprises aspartame and the gingerdiol compound or any of its comestibly acceptable salts. In some such embodiments, the introduction of the gingerdiol compound (or salt) permits one to use less aspartame (such as more than 10% less, more than 20% less, more than 30% less, more than 40% less, more than 50% less, more than 60% less, or more than 70% less) and still achieve a level of sweetness, umami, and/or kokumi characteristic of a comparable product that employs more aspartame. In some embodiments, the concentration of the gingerdiol compound, or its comestibly acceptable salts, is no more than 1000 ppm, or no more than 900 ppm, or no more than 800 ppm, or no more than 700 ppm, or no more than 600 ppm, or no more than 500 ppm, or no more than 400 ppm, or no more than 300 ppm, or no more than 200 ppm, or no more than 100 ppm, or no more than 50 ppm, or no more than 25 ppm, or no more than 10 ppm. Such ingestible compositions can be in any suitable form. In some embodiments, the ingestible composition is a food product, such as any of those specifically listed below. In other embodiments, the ingestible composition is a beverage product, such as a soda, and the like.

In certain particular embodiments, the ingestible composition comprises cyclamate and the gingerdiol compound or any of its comestibly acceptable salts. In some such embodiments, the introduction of the gingerdiol compound (or salt) permits one to use less cyclamate (such as more than 10% less, more than 20% less, more than 30% less, more than 40% less, more than 50% less, more than 60% less, or more than 70% less) and still achieve a level of sweetness, umami, and/or kokumi characteristic of a comparable product that employs more cyclamate. In some embodiments, the concentration of the gingerdiol compound, or its comestibly acceptable salts, is no more than 1000 ppm, or no more than 900 ppm, or no more than 800 ppm, or no more than 700 ppm, or no more than 600 ppm, or no more than 500 ppm, or no more than 400 ppm, or no more than 300 ppm, or no more than 200 ppm, or no more than 100 ppm, or no more than 50 ppm, or no more than 25 ppm, or no more than 10 ppm. Such ingestible compositions can be in any suitable form. In some embodiments, the ingestible composition is a food product, such as any of those specifically listed below. In other embodiments, the ingestible composition is a beverage product, such as a soda, and the like.

In certain particular embodiments, the ingestible composition comprises a mogroside (such as mogroside III, mogroside IV, mogroside V, siamenoside I, isomogroside V, mogroside IV_{E}, isomogroside IV_{E}, isomogroside IV, mogroside III_{E}, 11-oxomogroside V, the 1,6-alpha isomer of siamenoside I, and any combinations thereof) and the gingerdiol compound or any of its comestibly acceptable salts. In some such embodiments, the introduction of the gingerdiol compound (or salt) permits one to use less a mogroside (such as more than 10% less, more than 20% less, more than 30% less, more than 40% less, more than 50% less, more than 60% less, or more than 70% less) and still achieve a level of sweetness, umami, and/or kokumi characteristic of a comparable product that employs more mogroside. In some embodiments, the concentration of the gingerdiol compound, or its comestibly acceptable salts, is no more than 1000 ppm, or no more than 900 ppm, or no more than 800 ppm, or no more than 700 ppm, or no more than 600 ppm, or no more than 500 ppm, or no more than 400 ppm, or no more than 300 ppm, or no more than 200 ppm, or no more than 100 ppm, or no more than 50 ppm, or no more than 25 ppm, or no more than 10 ppm. Such ingestible compositions can be in any suitable form. In some embodiments, the ingestible composition is a food product, such as any of those specifically listed below. In other embodiments, the ingestible composition is a beverage product, such as a soda, and the like. Additional mogroside compounds that may be suitably used are described in U.S. Patent Application Publication No. 2017/0119032.

In some other aspects, the disclosure provides use of the gingerdiol compound, or a comestibly acceptable salt thereof, to enhance or confer an umami taste of an ingestible composition. In some embodiments, the concentration of the gingerdiol compound, or its comestibly acceptable salts, is no more than 1000 ppm, or no more than 900 ppm, or no more than 800 ppm, or no more than 700 ppm, or no more than 600 ppm, or no more than 500 ppm, or no more than 400 ppm, or no more than 300 ppm, or no more than 200 ppm, or no more than 100 ppm, or no more than 50 ppm, or no more than 25 ppm, or no more than 10 ppm, in the ingestible composition. Such ingestible compositions can be in any suitable form. In some embodiments, the ingestible composition is a food product, such as any of those specifically listed below.

In other embodiments, the ingestible composition is a beverage product, such as a soda, and the like.

In some other aspects, the disclosure provides use of the gingerdiol compound, or a comestibly acceptable salt thereof, to enhance or confer a kokumi taste of an ingestible composition. In some embodiments, the concentration of the gingerdiol compound, or its comestibly acceptable salts, is no more than 1000 ppm, or no more than 900 ppm, or no more than 800 ppm, or no more than 700 ppm, or no more than 600 ppm, or no more than 500 ppm, or no more than 400 ppm, or no more than 300 ppm, or no more than 200 ppm, or no more than 100 ppm, or no more than 50 ppm, or no more than 25 ppm, or no more than 10 ppm, in the ingestible composition. Such ingestible compositions can be in any suitable form. In some embodiments, the ingestible composition is a food product, such as any of those specifically listed below. In other embodiments, the ingestible composition is a beverage product, such as a soda, and the like.

In some other aspects, the disclosure provides use of the gingerdiol compound, or a comestibly acceptable salt thereof, to enhance or confer a salty taste of an ingestible composition. In some embodiments, the concentration of the gingerdiol compound, or its comestibly acceptable salts, is no more than 1000 ppm, or no more than 900 ppm, or no more than 800 ppm, or no more than 700 ppm, or no more than 600 ppm, or no more than 500 ppm, or no more than 400 ppm, or no more than 300 ppm, or no more than 200 ppm, or no more than 100 ppm, or no more than 50 ppm, or no more than 25 ppm, or no more than 10 ppm, in the ingestible composition. Such ingestible compositions can be in any suitable form. In some embodiments, the ingestible composition is a food product, such as any of those specifically listed below. In other embodiments, the ingestible composition is a beverage product, such as a soda, and the like.

In certain embodiments of any aspects and embodiments set forth herein that refer to an ingestible composition, the ingestible composition is a non-naturally-occurring product, such as a composition specifically manufactured for the production of a flavored product, such as food or beverage product.

In general, compounds as disclosed and described herein, individually or in combination, can be provided in a composition, such as an ingestible composition. In one embodiment, compounds as disclosed and described herein, individually or in combination, can impart a more sugar-like temporal profile or flavor profile to a sweetener composition by combining one or more of the compounds as disclosed and described herein with one or more sweeteners in the sweetener composition. In another embodiment, compounds as disclosed and described herein, individually or in combination, can increase or enhance the sweet taste of a composition by contacting the composition thereof with the compounds as disclosed and described herein to form a modified composition.

Thus, in some embodiments, the compositions set forth in any of the foregoing aspects (including in any uses or methods), comprise an gingerdiol compound (of any aspects or embodiments set forth herein) and a sweetener. In some embodiments, the composition further comprises a vehicle. In some embodiments, the vehicle is water. In some embodiments, the gingerdiol compound is present at a concentration at or below its umami or saltiness recognition threshold.

For example, in some embodiments, the sweetener (according to any of the embodiments set forth above) is present in an amount from about 0.1% to about 12% by weight. In some embodiments, the sweetener is present in an amount from about 0.2% to about 10% by weight. In some embodiments, the sweetener is present in an amount from about 0.3% to about 8% by weight. In some embodiments, the sweetener is present in an amount from about 0.4% to about 6% by weight. In some embodiments, the sweetener is present in an amount from about 0.5% to about 5% by weight. In some embodiments, the sweetener is present in an amount from about 1% to about 2% by weight. In some embodiments, the sweetener is present in an amount from about 0.1% to about 5% by weight. In some embodiments, the sweetener is present in an amount from about 0.1% to about 4% by weight. In some embodiments, the sweetener is present in an amount from about 0.1% to about 3% by weight. In some embodiments, the sweetener is present in an amount from about 0.1% to about 2% by weight. In some embodiments, the sweetener is present in an amount from about 0.1% to about 1% by weight. In some embodiments, the sweetener is present in an amount from about 0.1% to about 0.5% by weight. In some embodiments, the sweetener is present in an amount from about 0.5% to about 10% by weight. In some embodiments, the sweetener is present in an amount from about 2% to about 8% by weight. In some further embodiments of the embodiments set forth in this paragraph, the sweetener is sucrose, fructose, glucose, xylitol, erythritol, or combinations thereof.

In some other embodiments, the sweetener is present in an amount from 10 ppm to 1000 ppm. In some embodiments, the sweetener is present in an amount from 20 ppm to 800 ppm. In some embodiments, the sweetener is present in an amount from 30 ppm to 600 ppm. In some embodiments, the sweetener is present in an amount from 40 ppm to 500 ppm. In some embodiments, the sweetener is present in an amount from 50 ppm to 400 ppm. In some embodiments, the sweetener is present in an amount from 50 ppm to 300 ppm. In some embodiments, the sweetener is present in an amount from 50 ppm to 200 ppm. In some embodiments, the sweetener is present in an amount from 50 ppm to 150 ppm. In some further embodiments of the embodiments set forth in this paragraph, the sweetener is a steviol glycoside, a mogroside, a derivative of either of the foregoing, such as glycoside derivatives (e.g., glucosylates), or any combination thereof.

The compositions can include any suitable sweeteners or combination of sweeteners. In some embodiments, the sweetener is a common saccharide sweeteners, such as sucrose, fructose, glucose, and sweetener compositions comprising natural sugars, such as corn syrup (including high fructose corn syrup) or other syrups or sweetener concentrates derived from natural fruit and vegetable sources. In some embodiments, the sweetener is sucrose, fructose, or a combination thereof. In some embodiments, the sweetener is sucrose. In some other embodiments, the sweetener is selected from rare natural sugars including D-allose, D-psicose, L-ribose, D-tagatose, L-glucose, L-fucose, L-arabinose, D-turanose, and D-leucrose.

In some embodiments, the sweetener is selected from semi-synthetic “sugar alcohol” sweeteners such as erythritol, isomalt, lactitol, mannitol, sorbitol, xylitol, maltodextrin, and the like. In some embodiments, the sweetener is selected from artificial sweeteners such as aspartame, saccharin, acesulfame-K, cyclamate, sucralose, and alitame. In some embodiments, the sweetener is selected from the group consisting of cyclamic acid, mogroside, tagatose, maltose, galactose, mannose, sucrose, fructose, lactose, allulose, neotame and other aspartame derivatives, glucose, D-tryptophan, glycine, maltitol, lactitol, isomalt, hydrogenated glucose syrup (HGS), hydrogenated starch hydrolyzate (HSH), stevioside, rebaudioside A, other sweet Stevia-based glycosides, chemically modified steviol glycosides (such as glucosylated steviol glycosides), mogrosides, chemically modified mogrosides (such as glucosylated mogrosides), carrelame and other guanidine-based sweeteners. In some embodiments, the sweetener is a combination of two or more of the sweeteners set forth in this paragraph. In some embodiments, the sweetener may combinations of two, three, four or five sweeteners as disclosed herein. In some embodiments, the sweetener may be a sugar. In some embodiments, the sweetener may be a combination of one or more sugars and other natural and artificial sweeteners. In some embodiments, the sweetener is a sugar. In some embodiments, the sugar is cane sugar. In some embodiments, the sugar is beet sugar. In some embodiments, the sugar may be sucrose, fructose, glucose or combinations thereof. In some embodiments, the sugar may be sucrose. In some embodiments, the sugar may be a combination of fructose and glucose.

The sweetener can also include, for example, sweetener compositions comprising one or more natural or synthetic carbohydrate, such as corn syrup, high fructose corn syrup, high maltose corn syrup, glucose syrup, sucralose syrup, hydrogenated glucose syrup (HGS), hydrogenated starch hydrolyzate (HSH), or other syrups or sweetener concentrates derived from natural fruit and vegetable sources, or semi-synthetic “sugar alcohol” sweeteners such as polyols. Non-limiting examples of polyols in some embodiments include erythritol, maltitol, mannitol, sorbitol, lactitol, xylitol, isomalt, propylene glycol, glycerol (glycerin), threitol, galactitol, palatinose, reduced isomalto-oligosaccharides, reduced xylo-oligosaccharides, reduced gentio-oligosaccharides, reduced maltose syrup, reduced glucose syrup, isomaltulose, maltodextrin, and the like, and sugar alcohols or any other carbohydrates or combinations thereof capable of being reduced which do not adversely affect taste.

The sweetener may be a natural or synthetic sweetener that includes, but is not limited to, agave inulin, agave nectar, agave syrup, amazake, brazzein, brown rice syrup, coconut crystals, coconut sugars, coconut syrup, date sugar, fructans (also referred to as inulin fiber, fructo-oligosaccharides, or oligo-fructose), green stevia powder, Stevia rebaudiana, rebaudioside A, rebaudioside B, rebaudioside C, rebaudioside D, rebaudioside E, rebaudioside F, rebaudioside I, rebaudioside H, rebaudioside L, rebaudioside K, rebaudioside J, rebaudioside N, rebaudioside O, rebaudioside M and other sweet stevia-based glycosides, stevioside, stevioside extracts, honey, Jerusalem artichoke syrup, licorice root, luo han guo (fruit, powder, or extracts), lucuma (fruit, powder, or extracts), maple sap (including, for example, sap extracted from Acer saccharum, Acer nigrum, Acer rubrum, Acer saccharinum, Acer platanoides, Acer negundo, Acer macrophyllum, Acer grandidentatum, Acer glabrum, Acer mono), maple syrup, maple sugar, walnut sap (including, for example, sap extracted from Juglans cinerea, Juglans nigra, Juglans ailatifolia, Juglans regia), birch sap (including, for example, sap extracted from Betula papyrifera, Betula alleghaniensis, Betula lenta, Betula nigra, Betula populifolia, Betula pendula), sycamore sap (such as, for example, sap extracted from Platanus occidentalis), ironwood sap (such as, for example, sap extracted from Ostrya virginiana), mascobado, molasses (such as, for example, blackstrap molasses), molasses sugar, monatin, monellin, cane sugar (also referred to as natural sugar, unrefined cane sugar, or sucrose), palm sugar, panocha, piloncillo, rapadura, raw sugar, rice syrup, sorghum, sorghum syrup, cassava syrup (also referred to as tapioca syrup), thaumatin, yacon root, malt syrup, barley malt syrup, barley malt powder, beet sugar, cane sugar, crystalline juice crystals, caramel, carbitol, carob syrup, castor sugar, hydrogenated starch hydrolates, hydrolyzed can juice, hydrolyzed starch, invert sugar, anethole, arabinogalactan, arrope, syrup, P-4000, acesulfame potassium (also referred to as acesulfame K or ace-K), alitame (also referred to as aclame), advantame, aspartame, baiyunoside, neotame, benzamide derivatives, bernadame, canderel, carrelame and other guanidine-based sweeteners, vegetable fiber, corn sugar, coupling sugars, curculin, cyclamates, cyclocarioside I, demerara, dextran, dextrin, diastatic malt, dulcin, sucrol, valzin, dulcoside A, dulcoside B, emulin, enoxolone, maltodextrin, saccharin, estragole, ethyl maltol, glucin, gluconic acid, glucono-lactone, glucosamine, glucoronic acid, glycerol, glycine, glycyphillin, glycyrrhizin, glycyrrhetic acid monoglucuronide, golden sugar, yellow sugar, golden syrup, granulated sugar, gynostemma, hernandulcin, isomerized liquid sugars, jallab, chicory root dietary fiber, kynurenine derivatives (including N′-formyl-kynurenine, N′-acetyl-kynurenine, 6-chloro-kynurenine), galactitol, litesse, ligicane, lycasin, lugduname, guanidine, falernum, mabinlin I, mabinlin II, maltol, maltisorb, maltodextrin, maltotriol, mannosamine, miraculin, mizuame, mogrosides (including, for example, mogroside IV, mogroside V, and neomogroside), mukurozioside, nano sugar, naringin dihydrochalcone, neohesperidine dihydrochalcone, nib sugar, nigero-oligosaccharide, norbu, orgeat syrup, osladin, pekmez, pentadin, periandrin I, perillaldehyde, perillartine, petphyllum, phenylalanine, phlomisoside I, phlorodizin, phyllodulcin, polyglycitol syrups, polypodoside A, pterocaryoside A, pterocaryoside B, rebiana, refiners syrup, rub syrup, rubusoside, selligueain A, shugr, siamenoside I, siraitia grosvenorii, soybean oligosaccharide, Splenda, SRI oxime V, steviol glycoside, steviolbioside, stevioside, strogins 1, 2, and 4, sucronic acid, sucrononate, sugar, suosan, phloridzin, superaspartame, tetrasaccharide, threitol, treacle, trilobtain, tryptophan and derivatives (6-trifluoromethyl-tryptophan, 6-chloro-D-tryptophan), vanilla sugar, volemitol, birch syrup, aspartame-acesulfame, assugrin, and combinations or blends of any two or more thereof.

In still other embodiments, the sweetener can be a chemically or enzymatically modified natural high potency sweetener. Modified natural high potency sweeteners include glycosylated natural high potency sweetener such as glucosyl-, galactosyl-, or fructosyl-derivatives containing 1-50 glycosidic residues. Glycosylated natural high potency sweeteners may be prepared by enzymatic transglycosylation reaction catalyzed by various enzymes possessing transglycosylating activity. In some embodiments, the modified sweetener can be substituted or unsubstituted.

Additional sweeteners also include combinations of any two or more of any of the aforementioned sweeteners. In some embodiments, the sweetener may comprise combinations of two, three, four or five sweeteners as disclosed herein. In some embodiments, the sweetener may be a sugar. In some embodiments, the sweetener may be a combination of one or more sugars and other natural and artificial sweeteners. In some embodiments, the sweetener is a caloric sweetener, such as sucrose, fructose, xylitol, erythritol, or combinations thereof. In some embodiments, the ingestible compositions are free (or, in some embodiments) substantially free of stevia-derived sweeteners, such as steviol glycosides, glucosylated steviol glycosides, or rebaudiosides. For example, in some embodiments, the ingestible compositions are either free of stevia-derived sweeteners or comprise stevia-derived sweeteners in a concentration of no more than 1000 ppm, or no more than 500 ppm, or no more than 200 ppm, or no more than 100 ppm, or no more than 50 ppm, or no more than 20 ppm, or no more than 10 ppm, or no more than 5 ppm, or no more than 3 ppm, or no more than 1 ppm.

The gingerdiol compound can be present in the ingestible compositions in any suitable amount. In some embodiments, the gingerdiol compound are present in an amount sufficient to enhance the taste (e.g., enhance the umami, enhance the kokumi, enhance the saltiness, reduce the sourness, or reduce the bitterness) of the compositions. Thus, in some embodiments, the ingestible composition comprises the gingerdiol compounds in a concentration no greater than 200 ppm, or no greater than 150 ppm, or no greater than 100 ppm, or no greater than 50 ppm, or no greater than 40 ppm, or no greater than 30 ppm, or no greater than 20 ppm. In some embodiments, the gingerdiol compound is present in a minimum amount, such as 1 ppm or 5 ppm. Thus, in some embodiments, the ingestible composition comprises the gingerdiol compounds in a concentration ranging from 1 ppm to 200 ppm, or from 1 ppm to 150 ppm, or from 1 ppm to 100 ppm, or from 1 ppm to 50 ppm, or from 1 ppm to 40 ppm, or from 1 ppm to 30 ppm, or from 1 ppm to 20 ppm, or from 5 ppm to 200 ppm, or from 5 ppm to 150 ppm, or from 5 ppm to 100 ppm, or from 5 ppm to 50 ppm, or from 5 ppm to 40 ppm, or from 5 ppm to 30 ppm, or from 5 ppm to 20 ppm. In embodiments where a sweetener, such as sucrose or fructose, are present, the weight-to-weight ratio of sweetener to the gingerdiol compound in the ingestible composition ranges from 1000:1 to 50000:1, or from 1000:1 to 10000:1, or from 2000:1 to 8000:1.

The ingestible compositions or sweetener concentrates can, in certain embodiments, comprise any additional ingredients or combination of ingredients as are commonly used in food and beverage products, including, but not limited to: acids, including, for example citric acid, phosphoric acid, ascorbic acid, sodium acid sulfate, lactic acid, or tartaric acid; bitter ingredients, including, for example caffeine, quinine, green tea, catechins, polyphenols, green robusta coffee extract, green coffee extract, potassium chloride, menthol, or proteins (such as proteins and protein isolates derived from plants, algae, or fungi); coloring agents, including, for example caramel color, Red #40, Yellow #5, Yellow #6, Blue #1, Red #3, purple carrot, black carrot juice, purple sweet potato, vegetable juice, fruit juice, beta carotene, turmeric curcumin, or titanium dioxide; preservatives, including, for example sodium benzoate, potassium benzoate, potassium sorbate, sodium metabisulfate, sorbic acid, or benzoic acid; antioxidants including, for example ascorbic acid, calcium disodium EDTA, alpha tocopherols, mixed tocopherols, rosemary extract, grape seed extract, resveratrol, or sodium hexametaphosphate; vitamins or functional ingredients including, for example resveratrol, Co-Q10, omega 3 fatty acids, theanine, choline chloride (citocoline), fibersol, inulin (chicory root), taurine, panax ginseng extract, guanana extract, ginger extract, L-phenylalanine, L-carnitine, L-tartrate, D-glucoronolactone, inositol, bioflavonoids, Echinacea, ginko biloba, yerba mate, flax seed oil, garcinia cambogia rind extract, white tea extract, ribose, milk thistle extract, grape seed extract, pyrodixine HCl (vitamin B6), cyanoobalamin (vitamin B12), niacinamide (vitamin B3), biotin, calcium lactate, calcium pantothenate (pantothenic acid), calcium phosphate, calcium carbonate, chromium chloride, chromium polynicotinate, cupric sulfate, folic acid, ferric pyrophosphate, iron, magnesium lactate, magnesium carbonate, magnesium sulfate, monopotassium phosphate, monosodium phosphate, phosphorus, potassium iodide, potassium phosphate, riboflavin, sodium sulfate, sodium gluconate, sodium polyphosphate, sodium bicarbonate, thiamine mononitrate, vitamin D3, vitamin A palmitate, zinc gluconate, zinc lactate, or zinc sulphate; clouding agents, including, for example ester gun, brominated vegetable oil (BVO), or sucrose acetate isobutyrate (SAIB); buffers, including, for example sodium citrate, potassium citrate, or salt; flavors, including, for example propylene glycol, ethyl alcohol, glycerine, gum Arabic (gum acacia), maltodextrin, modified corn starch, dextrose, natural flavor, natural flavor with other natural flavors (natural flavor WONF), natural and artificial flavors, artificial flavor, silicon dioxide, magnesium carbonate, or tricalcium phosphate; or starches and stabilizers, including, for example pectin, xanthan gum, carboxylmethylcellulose (CMC), polysorbate 60, polysorbate 80, medium chain triglycerides, cellulose gel, cellulose gum, sodium caseinate, modified food starch, gum Arabic (gum acacia), inulin, or carrageenan.

The ingestible compositions or sweetener concentrates can have any suitable pH. In some embodiments, the gingerdiol compounds enhance the sweetness of a sweetener under a broad range of pH, e.g., from lower pH to neutral pH. The lower and neutral pH includes, but is not limited to, a pH from 1.5 to 9.0, or from 2.5 to 8.5; from 3.0 to 8.0; from 3.5 to 7.5; and from 4.0 to 7. In certain embodiments, compounds as disclosed and described herein, individually or in combination, can enhance the perceived sweetness of a fixed concentration of a sweetener in taste tests at a compound concentration of 50 μM, 40 μM, 30 μM, 20 μM, or 10 μM at both low to neutral pH value. In certain embodiments, the enhancement factor of the compounds as disclosed and described herein, individually or in combination, at the lower pH is substantially similar to the enhancement factor of the compounds at neutral pH. Such consistent sweet enhancing property under a broad range of pH allow a broad use in a wide variety of foods and beverages of the compounds as disclosed and described herein, individually or in combination.

The ingestible compositions set forth according to any of the foregoing embodiments, also include, in certain embodiments, one or more additional flavor-modifying compounds, such as compounds that enhance sweetness (e.g., hesperetin, naringenin, glucosylated steviol glycosides, etc.), compounds that block bitterness, compounds that enhance umami, compounds that reduce sourness or licorice taste, compounds that enhance saltiness, compounds that enhance a cooling effect, or any combinations of the foregoing.

Any salt that imparts a salty taste may be present or incorporated into a food product that contains a bioactive, taste modulating, or salty taste modulating compound of the present invention. The most commonly used salt for food applications is sodium chloride. Other illustrative sources of sodium salts that may be present of incorporated into a food product include sodium phosphates, mono sodium glutamate, sodium nitrite, sodium nitrate, sodium bicarbonate, sodium lactate, sodium citrate, and sodium stearoyl lactylate. Similar lithium, potassium, ammonium or other alkali earth salts may be present or included in addition or as an alternative to one or more sodium salts.

Thus, in some embodiments, ingestible compositions disclosed herein comprise the gingerdiol compound, or any comestibly acceptable salts thereof, according to any of the embodiments or combination of embodiments set forth above, are combined with one or more sweetness enhancing compounds. Such sweetness enhancing compounds include, but are not limited to, naturally derived compounds, such as hesperitin, naringenin, rhoifolin, glucosylated steviol glycosides, licorice-derived glucuronates, aromadendrin-3-O-acetate, or other like flavonols, or flavonoids, or synthetic compounds, such as any compounds set forth in U.S. Pat. Nos. 8,541,421; 8,815,956; 9,834,544; 8,592,592; 8,877,922; 9,000,054; and 9,000,051, as well as

U.S. Patent Application Publication No. 2017/0119032. The gingerdiol compounds (or comestibly acceptable salts thereof) may be used in combination with such other sweetness enhancers in any suitable ratio (w/w) ranging from 1:1000 to 1000:1, or from 1:100 to 100:1, or from, 1:50 to 50:1, or from 1:25 to 25:1, or from 1:10 to 10:1, such as 1:25, 1:24, 1:23, 1:22, 1:21, 1:20, 1:19, 1:18, 1:17, 1:16, 1:15, 1:14, 1:13, 1:12, 1:11, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, or 25:1. In some embodiments of any of the preceding embodiments, the gingerdiol compound (or any comestibly acceptable salts thereof) is combined with glucosylated steviol glycosides in any of the above ratios. As used herein, the term “glucosylated steviol glycoside” refers to the product of enzymatically glucosylating natural steviol glycoside compounds. The glucosylation generally occurs through a glycosidic bond, such as an α-1,2 bond, an α-1,4 bond, an α-1.6 bond, a β-1,2 bond, a β-1,4 bond, a β-1,6 bond, and so forth. In some embodiments of any of the preceding embodiments, the gingerdiol compound (or any comestibly acceptable salts thereof) is combined with 3-((4-amino-2,2-dioxo-1H-benzo[c][1,2,6]thiadiazin-5-yl)oxy)-2,2-dimethyl-N-propyl-propanamide, N-(1-((4-amino-2,2-dioxo-1H-benzo[c][1,2,6]thiadiazin-5-yl)oxy)-2-methyl-propan 2yl)isonicotinamide, or any combination thereof, in any of the above ratios.

In some further embodiments, ingestible compositions disclosed herein comprise the gingerdiol compound, or any comestibly acceptable salts thereof, according to any of the embodiments or combination of embodiments set forth above, are combined with one or more other umami or kokumi enhancing compounds. Such umami enhancing compounds include, but are not limited to, naturally derived compounds, such as ericamide, or synthetic compounds, such as any compounds set forth in U.S. Pat. Nos. 8,735,081; 8,124,121; and 8,968,708. The gingerdiol compound (or comestibly acceptable salts thereof) may be used in combination with such umami enhancers in any suitable ratio (w/w) ranging from 1:1000 to 1000:1, or from 1:100 to 100:1, or from, 1:50 to 50:1, or from 1:25 to 25:1, or from 1:10 to 10:1, such as 1:25, 1:24, 1:23, 1:22, 1:21, 1:20, 1:19, 1:18, 1:17, 1:16, 1:15, 1:14, 1:13, 1:12, 1:11, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, or 25:1.

In some further embodiments, ingestible compositions disclosed herein comprise the gingerdiol compound, or any comestibly acceptable salts thereof, according to any of the embodiments or combination of embodiments set forth above, are combined with one or more cooling enhancing compounds. Such cooling enhancing compounds include, but are not limited to, naturally derived compounds, such as menthol or analogs thereof, or synthetic compounds, such as any compounds set forth in U.S. Pat. Nos. 9,394,287 and 10,421,727. The gingerdiol compound (or comestibly acceptable salts thereof) may be used in combination with such umami enhancers in any suitable ratio (w/w) ranging from 1:1000 to 1000:1, or from 1:100 to 100:1, or from, 1:50 to 50:1, or from 1:25 to 25:1, or from 1:10 to 10:1, such as 1:25, 1:24, 1:23, 1:22, 1:21, 1:20, 1:19, 1:18, 1:17, 1:16, 1:15, 1:14, 1:13, 1:12, 1:11, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, or 25:1.

In some further embodiments, ingestible compositions disclosed herein comprise the gingerdiol compound, or any comestibly acceptable salts thereof, according to any of the embodiments or combination of embodiments set forth above, are combined with one or more bitterness blocking compounds. Such bitterness blocking compounds include, but are not limited to, naturally derived compounds, such as menthol or analogs thereof, or synthetic compounds, such as any compounds set forth in U.S.

U.S. Pat. Nos. 8,076,491; 8,445,692; and 9,247,759. The gingerdiol compound (or comestibly acceptable salts thereof) may be used in combination with such bitterness blockers in any suitable ratio (w/w) ranging from 1:1000 to 1000:1, or from 1:100 to 100:1, or from, 1:50 to 50:1, or from 1:25 to 25:1, or from 1:10 to 10:1, such as 1:25, 1:24, 1:23, 1:22, 1:21, 1:20, 1:19, 1:18, 1:17, 1:16, 1:15, 1:14, 1:13, 1:12, 1:11, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, or 25:1.

In some further embodiments, ingestible compositions disclosed herein comprise the gingerdiol compound, or any comestibly acceptable salts thereof, according to any of the embodiments or combination of embodiments set forth above, are combined with one or more sour taste modulating compounds. The gingerdiol compound (or comestibly acceptable salts thereof) may be used in combination with such sour taste modulating compounds in any suitable ratio (w/w) ranging from 1:1000 to 1000:1, or from 1:100 to 100:1, or from, 1:50 to 50:1, or from 1:25 to 25:1, or from 1:10 to 10:1, such as 1:25, 1:24, 1:23, 1:22, 1:21, 1:20, 1:19, 1:18, 1:17, 1:16, 1:15, 1:14, 1:13, 1:12, 1:11, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, or 25:1.

In some further embodiments, ingestible compositions disclosed herein comprise the gingerdiol compound, or any comestibly acceptable salts thereof, according to any of the embodiments or combination of embodiments set forth above, are combined with one or more mouthfeel modifying compounds. Such mouthfeel modifying compounds include, but are not limited to, tannins, cellulosic materials, bamboo powder, and the like. The gingerdiol compound (or comestibly acceptable salts thereof) may be used in combination with such mouthfeel enhancers in any suitable ratio (w/w) ranging from 1:1000 to 1000:1, or from 1:100 to 100:1, or from, 1:50 to 50:1, or from 1:25 to 25:1, or from 1:10 to 10:1, such as 1:25, 1:24, 1:23, 1:22, 1:21, 1:20, 1:19, 1:18, 1:17, 1:16, 1:15, 1:14, 1:13, 1:12, 1:11, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, or 25:1.

In some further embodiments, ingestible compositions disclosed herein comprise the gingerdiol compound, or any comestibly acceptable salts thereof, according to any of the embodiments or combination of embodiments set forth above, are combined with one or more flavor masking compounds. Such flavor masking compounds include, but are not limited to, cellulosic materials, materials extracted from fungus, materials extracted from plants, citric acid, carbonic acid (or carbonates), and the like. The gingerdiol compound (or comestibly acceptable salts thereof) may be used in combination with such mouthfeel enhancers in any suitable ratio (w/w) ranging from 1:1000 to 1000:1, or from 1:100 to 100:1, or from, 1:50 to 50:1, or from 1:25 to 25:1, or from 1:10 to 10:1, such as 1:25, 1:24, 1:23, 1:22, 1:21, 1:20, 1:19, 1:18, 1:17, 1:16, 1:15, 1:14, 1:13, 1:12, 1:11, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, or 25:1.

In some aspects related to the preceding aspects and embodiments, the disclosure provides uses of the gingerdiol compound (or comestibly acceptable salts thereof) to enhance the flavor of a flavored composition, such as a flavored article. Such flavored compositions can use any suitable flavors, such as fruit flavors, meat flavors, vegetable flavors, and the like. In some embodiments, the flavored composition is a soup or broth, or a chip, or a beverage.

### Flavored Products and Concentrates

In certain aspects, the disclosure provides flavored products comprising any compositions of the preceding aspects or embodiments thereof. In some embodiments, the flavored products are beverage products, such as soda, flavored water, tea, and the like. In some other embodiments, the flavored products are food products, such as yogurt.

In embodiments where the flavored product is a beverage, the beverage may be selected from the group consisting of enhanced sparkling beverages, colas, lemon-lime flavored sparkling beverages, orange flavored sparkling beverages, grape flavored sparkling beverages, strawberry flavored sparkling beverages, pineapple flavored sparkling beverages, ginger-ales, root beers, fruit juices, fruit-flavored juices, juice drinks, nectars, vegetable juices, vegetable-flavored juices, sports drinks, energy drinks, enhanced water drinks, enhanced water with vitamins, near water drinks, coconut waters, tea type drinks, coffees, cocoa drinks, beverages containing milk components, beverages containing cereal extracts and smoothies. In some embodiments, the beverage may be a soft drink.

In certain embodiments of any aspects and embodiments set forth herein that refer to a flavored product, the flavored product is a non-naturally-occurring product, such as a packaged food or beverage product.

Further non-limiting examples of food and beverage products or formulations include sweet coatings, frostings, or glazes for such products or any entity included in the Soup category, the Dried Processed Food category, the Beverage category, the Ready Meal category, the Canned or Preserved Food category, the Frozen Processed Food category, the Chilled Processed Food category, the Snack Food category, the Baked Goods category, the Confectionery category, the Dairy Product category, the Ice Cream category, the Meal Replacement category, the Pasta and Noodle category, and the Sauces, Dressings, Condiments category, the Baby Food category, and/or the Spreads category.

In general, the Soup category refers to canned/preserved, dehydrated, instant, chilled, UHT and frozen soup. For the purpose of this definition soup(s) means a food prepared from meat, poultry, fish, vegetables, grains, fruit and other ingredients, cooked in a liquid which may include visible pieces of some or all of these ingredients. It may be clear (as a broth) or thick (as a chowder), smooth, pureed or chunky, ready-to-serve, semi-condensed or condensed and may be served hot or cold, as a first course or as the main course of a meal or as a between meal snack (sipped like a beverage). Soup may be used as an ingredient for preparing other meal components and may range from broths (consommé) to sauces (cream or cheese-based soups).

The Dehydrated and Culinary Food Category usually means: (i) Cooking aid products such as: powders, granules, pastes, concentrated liquid products, including concentrated bouillon, bouillon and bouillon like products in pressed cubes, tablets or powder or granulated form, which are sold separately as a finished product or as an ingredient within a product, sauces and recipe mixes (regardless of technology); (ii) Meal solutions products such as: dehydrated and freeze dried soups, including dehydrated soup mixes, dehydrated instant soups, dehydrated ready-to-cook soups, dehydrated or ambient preparations of ready-made dishes, meals and single serve entrees including pasta, potato and rice dishes; and (iii) Meal embellishment products such as: condiments, marinades, salad dressings, salad toppings, dips, breading, batter mixes, shelf stable spreads, barbecue sauces, liquid recipe mixes, concentrates, sauces or sauce mixes, including recipe mixes for salad, sold as a finished product or as an ingredient within a product, whether dehydrated, liquid or frozen.

The Beverage category usually means beverages, beverage mixes and concentrates, including but not limited to, carbonated and non-carbonated beverages, alcoholic and non-alcoholic beverages, ready to drink beverages, liquid concentrate formulations for preparing beverages such as sodas, and dry powdered beverage precursor mixes. The Beverage category also includes the alcoholic drinks, the soft drinks, sports drinks, isotonic beverages, and hot drinks. The alcoholic drinks include, but are not limited to beer, cider/perry, FABs, wine, and spirits. The soft drinks include, but are not limited to carbonates, such as colas and non-cola carbonates; fruit juice, such as juice, nectars, juice drinks and fruit flavored drinks; bottled water, which includes sparkling water, spring water and purified/table water; functional drinks, which can be carbonated or still and include sport, energy or elixir drinks; concentrates, such as liquid and powder concentrates in ready to drink measure. The drinks, either hot or cold, include, but are not limited to coffee or ice coffee, such as fresh, instant, and combined coffee; tea or ice tea, such as black, green, white, oolong, and flavored tea; and other drinks including flavor-, malt- or plant-based powders, granules, blocks or tablets mixed with milk or water.

The Snack Food category generally refers to any food that can be a light informal meal including, but not limited to Sweet and savory snacks and snack bars. Examples of snack food include, but are not limited to fruit snacks, chips/crisps, extruded snacks, tortilla/corn chips, popcorn, pretzels, nuts and other sweet and savory snacks. Examples of snack bars include, but are not limited to granola/muesli bars, breakfast bars, energy bars, fruit bars and other snack bars.

The Baked Goods category generally refers to any edible product the process of preparing which involves exposure to heat or excessive sunlight. Examples of baked goods include, but are not limited to bread, buns, cookies, muffins, cereal, toaster pastries, pastries, waffles, tortillas, biscuits, pies, bagels, tarts, quiches, cake, any baked foods, and any combination thereof.

The Ice Cream category generally refers to frozen dessert containing cream and sugar and flavoring. Examples of ice cream include, but are not limited to: impulse ice cream; take-home ice cream; frozen yoghurt and artisanal ice cream; soy, oat, bean (e.g., red bean and mung bean), and rice-based ice creams.

The Confectionery category generally refers to edible product that is sweet to the taste. Examples of confectionery include, but are not limited to candies, gelatins, chocolate confectionery, sugar confectionery, gum, and the likes and any combination products.

The Meal Replacement category generally refers to any food intended to replace the normal meals, particularly for people having health or fitness concerns. Examples of meal replacement include, but are not limited to slimming products and convalescence products.

The Ready Meal category generally refers to any food that can be served as meal without extensive preparation or processing. The ready meal includes products that have had recipe “skills” added to them by the manufacturer, resulting in a high degree of readiness, completion and convenience. Examples of ready meal include, but are not limited to canned/preserved, frozen, dried, chilled ready meals; dinner mixes; frozen pizza; chilled pizza; and prepared salads.

The Pasta and Noodle category includes any pastas and/or noodles including, but not limited to canned, dried and chilled/fresh pasta; and plain, instant, chilled, frozen and snack noodles.

The Canned/Preserved Food category includes, but is not limited to canned/preserved meat and meat products, fish/seafood, vegetables, tomatoes, beans, fruit, ready meals, soup, pasta, and other canned/preserved foods.

The Frozen Processed Food category includes, but is not limited to frozen processed red meat, processed poultry, processed fish/seafood, processed vegetables, meat substitutes, processed potatoes, bakery products, desserts, ready meals, pizza, soup, noodles, and other frozen food.

The Dried Processed Food category includes, but is not limited to rice, dessert mixes, dried ready meals, dehydrated soup, instant soup, dried pasta, plain noodles, and instant noodles. The Chill Processed Food category includes, but is not limited to chilled processed meats, processed fish/seafood products, lunch kits, fresh cut fruits, ready meals, pizza, prepared salads, soup, fresh pasta and noodles.

The Sauces, Dressings and Condiments category includes, but is not limited to tomato pastes and purees, bouillon/stock cubes, herbs and spices, monosodium glutamate (MSG), table sauces, soy based sauces, pasta sauces, wet/cooking sauces, dry sauces/powder mixes, ketchup, mayonnaise, mustard, salad dressings, vinaigrettes, dips, pickled products, and other sauces, dressings and condiments.

The Baby Food category includes, but is not limited to milk- or soybean-based formula; and prepared, dried and other baby food.

The Spreads category includes, but is not limited to jams and preserves, honey, chocolate spreads, nut based spreads, and yeast based spreads.

The Dairy Product category generally refers to edible product produced from mammal's milk. Examples of dairy product include, but are not limited to drinking milk products, cheese, yoghurt and sour milk drinks, and other dairy products.

Additional examples for flavored products, particularly food and beverage products or formulations, are provided as follows. Exemplary ingestible compositions include one or more confectioneries, chocolate confectionery, tablets, countlines, bagged selflines/softlines, boxed assortments, standard boxed assortments, twist wrapped miniatures, seasonal chocolate, chocolate with toys, alfajores, other chocolate confectionery, mints, standard mints, power mints, boiled sweets, pastilles, gums, jellies and chews, toffees, caramels and nougat, medicated confectionery, lollipops, liquorice, other sugar confectionery, bread, packaged/industrial bread, unpackaged/artisanal bread, pastries, cakes, packaged/industrial cakes, unpackaged/artisanal cakes, cookies, chocolate coated biscuits, sandwich biscuits, filled biscuits, savory biscuits and crackers, bread substitutes, breakfast cereals, rte cereals, family breakfast cereals, flakes, muesli, other cereals, children's breakfast cereals, hot cereals, ice cream, impulse ice cream, single portion dairy ice cream, single portion water ice cream, multi-pack dairy ice cream, multi-pack water ice cream, take-home ice cream, take-home dairy ice cream, ice cream desserts, bulk ice cream, take-home water ice cream, frozen yoghurt, artisanal ice cream, dairy products, milk, fresh/pasteurized milk, full fat fresh/pasteurized milk, semi skimmed fresh/pasteurized milk, long-life/uht milk, full fat long life/uht milk, semi skimmed long life/uht milk, fat-free long life/uht milk, condensed/evaporated milk, plain condensed/evaporated milk, flavored, functional and other condensed milk, flavored milk drinks, dairy only flavored milk drinks, flavored milk drinks with fruit juice, soy milk, sour milk drinks, fermented dairy drinks, coffee whiteners, powder milk, flavored powder milk drinks, cream, cheese, processed cheese, spreadable processed cheese, unspreadable processed cheese, unprocessed cheese, spreadable unprocessed cheese, hard cheese, packaged hard cheese, unpackaged hard cheese, yoghurt, plain/natural yoghurt, flavored yoghurt, fruited yoghurt, probiotic yoghurt, drinking yoghurt, regular drinking yoghurt, probiotic drinking yoghurt, chilled and shelf-stable desserts, dairy-based desserts, soy-based desserts, chilled snacks, fromage frais and quark, plain fromage frais and quark, flavored fromage frais and quark, savory fromage frais and quark, sweet and savory snacks, fruit snacks, chips/crisps, extruded snacks, tortilla/corn chips, popcorn, pretzels, nuts, other sweet and savory snacks, snack bars, granola bars, breakfast bars, energy bars, fruit bars, other snack bars, meal replacement products, slimming products, convalescence drinks, ready meals, canned ready meals, frozen ready meals, dried ready meals, chilled ready meals, dinner mixes, frozen pizza, chilled pizza, soup, canned soup, dehydrated soup, instant soup, chilled soup, hot soup, frozen soup, pasta, canned pasta, dried pasta, chilled/fresh pasta, noodles, plain noodles, instant noodles, cups/bowl instant noodles, pouch instant noodles, chilled noodles, snack noodles, canned food, canned meat and meat products, canned fish/seafood, canned vegetables, canned tomatoes, canned beans, canned fruit, canned ready meals, canned soup, canned pasta, other canned foods, frozen food, frozen processed red meat, frozen processed poultry, frozen processed fish/seafood, frozen processed vegetables, frozen meat substitutes, frozen potatoes, oven baked potato chips, other oven baked potato products, non-oven frozen potatoes, frozen bakery products, frozen desserts, frozen ready meals, frozen pizza, frozen soup, frozen noodles, other frozen food, dried food, dessert mixes, dried ready meals, dehydrated soup, instant soup, dried pasta, plain noodles, instant noodles, cups/bowl instant noodles, pouch instant noodles, chilled food, chilled processed meats, chilled fish/seafood products, chilled processed fish, chilled coated fish, chilled smoked fish, chilled lunch kit, chilled ready meals, chilled pizza, chilled soup, chilled/fresh pasta, chilled noodles, oils and fats, olive oil, vegetable and seed oil, cooking fats, butter, margarine, spreadable oils and fats, functional spreadable oils and fats, sauces, dressings and condiments, tomato pastes and purees, bouillon/stock cubes, stock cubes, gravy granules, liquid stocks and fonds, herbs and spices, fermented sauces, soy based sauces, pasta sauces, wet sauces, dry sauces/powder mixes, ketchup, mayonnaise, regular mayonnaise, mustard, salad dressings, regular salad dressings, low fat salad dressings, vinaigrettes, dips, pickled products, other sauces, dressings and condiments, baby food, milk formula, standard milk formula, follow-on milk formula, toddler milk formula, hypoallergenic milk formula, prepared baby food, dried baby food, other baby food, spreads, jams and preserves, honey, chocolate spreads, nut-based spreads, and yeast-based spreads. Exemplary ingestible compositions also include confectioneries, bakery products, ice creams, dairy products, sweet and savory snacks, snack bars, meal replacement products, ready meals, soups, pastas, noodles, canned foods, frozen foods, dried foods, chilled foods, oils and fats, baby foods, or spreads or a mixture thereof. Exemplary ingestible compositions also include breakfast cereals, sweet beverages or solid or liquid concentrate compositions for preparing beverages, ideally so as to enable the reduction in concentration of previously known saccharide sweeteners, or artificial sweeteners.

Some embodiments provide a chewable composition that may or may not be intended to be swallowed. In some embodiments, the chewable composition may be gum, chewing gum, sugarized gum, sugar-free gum, functional gum, bubble gum including compounds as disclosed and described herein, individually or in combination.

Typically at least a sweet receptor modulating amount, a sweet receptor ligand modulating amount, a sweet flavor modulating amount, a sweet flavoring agent amount, a sweet flavor enhancing amount, or a therapeutically effective amount of one or more of the present compounds will be added to the ingestible composition, optionally in the presence of sweeteners so that the sweet flavor modified ingestible composition has an increased sweet taste as compared to the ingestible composition prepared without the compounds of the present invention, as judged by human beings or animals in general, or in the case of formulations testing, as judged by a majority of a panel of at least eight human taste testers, via procedures commonly known in the field.

In some embodiments, compounds as disclosed and described herein, individually or in combination, modulate the sweet taste or other taste properties of other natural or synthetic sweet tastants, and ingestible compositions made therefrom. In one embodiment, the compounds as disclosed and described herein, individually or in combination, may be used or provided in its ligand enhancing concentration(s). For example, the compounds as disclosed and described herein, individually or in combination, may be present in an amount of from 0.001 ppm to 100 ppm, or narrower alternative ranges from 0.1 ppm to 50 ppm, from 0.01 ppm to 40 ppm, from 0.05 ppm to 30 ppm, from 0.01 ppm to 25 ppm, or from 0.1 ppm to 30 ppm, or from 0.1 ppm to 25 ppm, or from 1 ppm to 30 ppm, or from 1 ppm to 25 ppm.

In some embodiments, gingerdiol compounds as disclosed and described herein, individually or in combination, may be provided in a flavoring concentrate formulation, e.g., suitable for subsequent processing to produce a ready-to-use (i.e., ready-to-serve) product. By “a flavoring concentrate formulation”, it is meant a formulation which should be reconstituted with one or more diluting medium to become a ready-to-use composition. The term “ready-to-use composition” is used herein interchangeably with “ingestible composition”, which denotes any substance that, either alone or together with another substance, can be taken by mouth whether intended for consumption or not. In one embodiment, the ready-to-use composition includes a composition that can be directly consumed by a human or animal. The flavoring concentrate formulation is typically used by mixing with or diluted by one or more diluting medium, e.g., any consumable or ingestible ingredient or product, to impart or modify one or more flavors to the diluting medium. Such a use process is often referred to as reconstitution. The reconstitution can be conducted in a household setting or an industrial setting. For example, a frozen fruit juice concentrate can be reconstituted with water or other aqueous medium by a consumer in a kitchen to obtain the ready-to-use fruit juice beverage. In another example, a soft drink syrup concentrate can be reconstituted with water or other aqueous medium by a manufacturer in large industrial scales to produce the ready-to-use soft drinks. Since the flavoring concentrate formulation has the flavoring agent or flavor modifying agent in a concentration higher than the ready-to-use composition, the flavoring concentrate formulation is typically not suitable for being consumed directly without reconstitution. There are many benefits of using and producing a flavoring concentrate formulation. For example, one benefit is the reduction in weight and volume for transportation as the flavoring concentrate formulation can be reconstituted at the time of usage by the addition of suitable solvent, solid or liquid.

The flavored products set forth according to any of the foregoing embodiments, also include, in certain embodiments, one or more additional flavor-modifying compounds, such as compounds that enhance sweetness (e.g., hesperetin, naringenin, glucosylated steviol glycosides, etc.), compounds that block bitterness, compounds that enhance umami, compounds that reduce sourness, compounds that enhance saltiness, compounds that enhance a cooling effect, or any combinations of the foregoing.

In certain embodiments of any aspects and embodiments set forth herein that refer to a sweetening or flavoring concentrate, the sweetening or flavoring concentrate is a non-naturally-occurring product, such as a composition specifically manufactured for the production of a flavored product, such as food or beverage product.

In one embodiment, the flavoring concentrate formulation comprises i) compounds as disclosed and described herein, individually or in combination; ii) a carrier; and iii) optionally at least one adjuvant. The term “carrier” denotes a usually inactive accessory substance, such as solvents, binders, or other inert medium, which is used in combination with the present compound and one or more optional adjuvants to form the formulation. For example, water or starch can be a carrier for a flavoring concentrate formulation. In some embodiments, the carrier is the same as the diluting medium for reconstituting the flavoring concentrate formulation; and in other embodiments, the carrier is different from the diluting medium. The term “carrier” as used herein includes, but is not limited to, ingestibly acceptable carrier.

The term “adjuvant” denotes an additive which supplements, stabilizes, maintains, or enhances the intended function or effectiveness of the active ingredient, such as the compound of the present invention. In one embodiment, the at least one adjuvant comprises one or more flavoring agents. The flavoring agent may be of any flavor known to one skilled in the art or consumers, such as the flavor of chocolate, coffee, tea, mocha, French vanilla, peanut butter, chai, or combinations thereof. In another embodiment, the at least one adjuvant comprises one or more sweeteners. The one or more sweeteners can be any of the sweeteners described in this application. In another embodiment, the at least one adjuvant comprises one or more ingredients selected from the group consisting of a emulsifier, a stabilizer, an antimicrobial preservative, an antioxidant, vitamins, minerals, fats, starches, protein concentrates and isolates, salts, and combinations thereof. Examples of emulsifiers, stabilizers, antimicrobial preservatives, antioxidants, vitamins, minerals, fats, starches, protein concentrates and isolates, and salts are described in U.S. Pat. No. 6,468,576, the content of which is hereby incorporated by reference in its entirety for all purposes.

In one embodiment, the present flavoring concentrate formulation can be in a form selected from the group consisting of liquid including solution and suspension, solid, foamy material, paste, gel, cream, and a combination thereof, such as a liquid containing certain amount of solid contents. In one embodiment, the flavoring concentrate formulation is in form of a liquid including aqueous-based and nonaqueous-based. In some embodiments, the present flavoring concentrate formulation can be carbonated or non-carbonated.

The flavoring concentrate formulation may further comprise a freezing point depressant, nucleating agent, or both as the at least one adjuvant. The freezing point depressant is an ingestibly acceptable compound or agent which can depress the freezing point of a liquid or solvent to which the compound or agent is added. That is, a liquid or solution containing the freezing point depressant has a lower freezing point than the liquid or solvent without the freezing point depressant. In addition to depress the onset freezing point, the freezing point depressant may also lower the water activity of the flavoring concentrate formulation. The examples of the freezing point depressant include, but are not limited to, carbohydrates, oils, ethyl alcohol, polyol, e.g., glycerol, and combinations thereof. The nucleating agent denotes an ingestibly acceptable compound or agent which is able to facilitate nucleation. The presence of nucleating agent in the flavoring concentrate formulation can improve the mouthfeel of the frozen Blushes of a frozen slush and to help maintain the physical properties and performance of the slush at freezing temperatures by increasing the number of desirable ice crystallization centers. Examples of nucleating agents include, but are not limited to, calcium silicate, calcium carbonate, titanium dioxide, and combinations thereof.

In one embodiment, the flavoring concentrate formulation is formulated to have a low water activity for extended shelf life. Water activity is the ratio of the vapor pressure of water in a formulation to the vapor pressure of pure water at the same temperature. In one embodiment, the flavoring concentrate formulation has a water activity of less than about 0.85. In another embodiment, the flavoring concentrate formulation has a water activity of less than about 0.80. In another embodiment, the flavoring concentrate formulation has a water activity of less than about 0.75.

In one embodiment, the flavoring concentrate formulation has the present compound in a concentration that is at least 2 times of the concentration of the compound in a ready-to-use composition. In one embodiment, the flavoring concentrate formulation has the present compound in a concentration that is at least 5 times of the concentration of the compound in a ready-to-use composition. In one embodiment, the flavoring concentrate formulation has the present compound in a concentration that is at least 10 times of the concentration of the compound in a ready-to-use composition. In one embodiment, the flavoring concentrate formulation has the present compound in a concentration that is at least 15 times of the concentration of the compound in a ready-to-use composition. In one embodiment, the flavoring concentrate formulation has the present compound in a concentration that is at least 20 times of the concentration of the compound in a ready-to-use composition. In one embodiment, the flavoring concentrate formulation has the present compound in a concentration that is at least 30 times of the concentration of the compound in a ready-to-use composition. In one embodiment, the flavoring concentrate formulation has the present compound in a concentration that is at least 40 times of the concentration of the compound in a ready-to-use composition. In one embodiment, the flavoring concentrate formulation has the present compound in a concentration that is at least 50 times of the concentration of the compound in a ready-to-use composition. In one embodiment, the flavoring concentrate formulation has the present compound in a concentration that is at least 60 times of the concentration of the compound in a ready-to-use composition. In one embodiment, the flavoring concentrate formulation has the present compound in a concentration that is up to 100 times of the concentration of the compound in a ready-to-use composition.

The sweetening or flavoring concentrates set forth according to any of the foregoing embodiments, also include, in certain embodiments, one or more additional flavor-modifying compounds, such as compounds that enhance sweetness (e.g., hesperetin, naringenin, glucosylated steviol glycosides, etc.), compounds that block bitterness (e.g., eriodictyol, homoeriodictyol, sterubin, and salts or glycoside derivatives thereof, as well as vanillyl lignans, e.g., matairesinol and other compounds set forth in PCT Publication No. WO 2012/146584), compounds that enhance umami (e.g., rubemamine, rubescenamine, (E)-3-(3,4-dimethoxyphenyl)-N-(4-methoxyphenethyl) acrylamide, and the like), compounds that reduce sourness and/or licorice taste, compounds that enhance saltiness, compounds that enhance a cooling effect, or any combinations of the foregoing.

### Tabletop Flavoring Compositions

In some further aspects, the disclosure provides a tabletop flavoring composition comprising: (a) an gingerdiol compound (according to any aspects and embodiments set forth herein), or a comestibly acceptable salt thereof; and (b) at least one bulking agent.

The tabletop flavoring composition may take any suitable form including, but not limited to, an amorphous solid, a crystal, a powder, a tablet, a liquid, a cube, a glace or coating, a granulated product, an encapsulated form abound to or coated on to carriers/particles, wet or dried, or combinations thereof.

The tabletop flavoring composition may contain further additives known to those skilled in the art. These additives include but are not limited to bubble forming agents, bulking agents, carriers, fibers, sugar alcohols, oligosaccharides, sugars, high intensity sweeteners, nutritive sweeteners, flavorings, flavor enhancers, flavor stabilizers, acidulants, anti-caking and free-flow agents. Such additives are for example described by H. Mitchell (H. Mitchell, “Sweeteners and Sugar Alternatives in Food Technology”, Blackwell Publishing Ltd, 2006, which is incorporated herein by reference in its entirety). As used herein, the term “flavorings” may include those flavors known to the skilled person, such as natural and artificial flavors. These flavorings may be chosen from synthetic flavor oils and flavoring aromatics and/or oils, oleoresins and extracts derived from plants, leaves, flowers, fruits, and so forth, and combinations thereof. Non-limiting representative flavor oils include spearmint oil, cinnamon oil, oil of wintergreen (methyl salicylate), peppermint oil, Japanese mint oil, clove oil, bay oil, anise oil, eucalyptus oil, thyme oil, cedar leaf oil, oil of nutmeg, allspice, oil of sage, mace, oil of bitter almonds, and cassia oil. Also useful flavorings are artificial, natural and synthetic fruit flavors such as vanilla, and citrus oils including lemon, orange, lime, grapefruit, yazu, sudachi, and fruit essences including apple, pear, peach, grape, blueberry, strawberry, raspberry, cherry, plum, pineapple, watermelon, apricot, banana, melon, apricot, ume, cherry, raspberry, blackberry, tropical fruit, mango, mangosteen, pomegranate, papaya and so forth. Other potential flavors include a milk flavor, a butter flavor, a cheese flavor, a cream flavor, and a yogurt flavor; a vanilla flavor; tea or coffee flavors, such as a green tea flavor, a oolong tea flavor, a tea flavor, a cocoa flavor, a chocolate flavor, and a coffee flavor; mint flavors, such as a peppermint flavor, a spearmint flavor, and a Japanese mint flavor; spicy flavors, such as an asafetida flavor, an ajowan flavor, an anise flavor, an angelica flavor, a fennel flavor, an allspice flavor, a cinnamon flavor, a camomile flavor, a mustard flavor, a cardamom flavor, a caraway flavor, a cumin flavor, a clove flavor, a pepper flavor, a coriander flavor, a sassafras flavor, a savory flavor, a Zanthoxyli Fructus flavor, a perilla flavor, a juniper berry flavor, a ginger flavor, a star anise flavor, a horseradish flavor, a thyme flavor, a tarragon flavor, a dill flavor, a capsicum flavor, a nutmeg flavor, a basil flavor, a marjoram flavor, a rosemary flavor, a bayleaf flavor, and a wasabi (Japanese horseradish) flavor; alcoholic flavors, such as a wine flavor, a whisky flavor, a brandy flavor, a rum flavor, a gin flavor, and a liqueur flavor; floral flavors; and vegetable flavors, such as an onion flavor, a garlic flavor, a cabbage flavor, a carrot flavor, a celery flavor, mushroom flavor, and a tomato flavor. These flavoring agents may be used in liquid or solid form and may be used individually or in admixture. Commonly used flavors include mints such as peppermint, menthol, spearmint, artificial vanilla, cinnamon derivatives, and various fruit flavors, whether employed individually or in admixture. Flavors may also provide breath freshening properties, particularly the mint flavors when used in combination with cooling agents.

Flavors may also provide breath freshening properties, particularly the mint flavors when used in combination with cooling agents. These flavorings may be used in liquid or solid form and may be used individually or in admixture. Other useful flavorings include aldehydes and esters such as cinnamyl acetate, cinnamaldehyde, citral diethylacetal, dihydrocarvyl acetate, eugenyl formate, p-methylamisol, and so forth may be used. Generally any flavoring or food additive such as those described in Chemicals Used in Food Processing, publication 1274, pages 63-258, by the National Academy of Sciences, may be used. This publication is incorporated herein by reference.

Further examples of aldehyde flavorings include but are not limited to acetaldehyde (apple), benzaldehyde (cherry, almond), anisic aldehyde (licorice, anise), cinnamic aldehyde (cinnamon), citral, i.e., alpha-citral (lemon, lime), neral, i.e., beta-citral (lemon, lime), decanal (orange, lemon), ethyl vanillin (vanilla, cream), heliotrope, i.e., piperonal (vanilla, cream), vanillin (vanilla, cream), alpha-amyl cinnamaldehyde (spicy fruity flavors), butyraldehyde (butter, cheese), valeraldehyde (butter, cheese), citronellal (modifies, many types), decanal (citrus fruits), aldehyde C-8 (citrus fruits), aldehyde C-9 (citrus fruits), aldehyde C-12 (citrus fruits), 2-ethyl butyraldehyde (berry fruits), hexenal, i.e., trans-2 (berry fruits), tolyl aldehyde (cherry, almond), veratraldehyde (vanilla), 2,6-dimethyl-5-heptenal, i.e., melonal (melon), 2,6-dimethyloctanal (green fruit), and 2-dodecenal (citrus, mandarin), cherry, grape, strawberry shortcake, and mixtures thereof. These listings of flavorings are merely exemplary and are not meant to limit either the term “flavoring” or the scope of the disclosure generally.

In some embodiments, the flavoring may be employed in either liquid form and/or dried form. When employed in the latter form, suitable drying means such as spray drying the oil may be used. Alternatively, the flavoring may be absorbed onto water soluble materials, such as cellulose, starch, sugar, maltodextrin, gum arabic and so forth or may be encapsulated. The actual techniques for preparing such dried forms are well-known.

In some embodiments, the tabletop sweetener can be made to be similar to brown sugar. In such embodiments, compounds imparting brown notes can be added to the composition to make it taste more similar to brown sugar.

In some embodiments, the flavorings may be used in many distinct physical forms well-known in the art to provide an initial burst of flavor and/or a prolonged sensation of flavor. Without being limited thereto, such physical forms include free forms, such as spray dried, powdered, beaded forms, encapsulated forms, and mixtures thereof.

Suitable bulking agents include, but are not limited to maltodextrin (10 DE, 18 DE, or 5 DE), corn syrup solids (20 or 36 DE), sucrose, fructose, glucose, invert sugar, sorbitol, xylose, ribulose, mannose, xylitol, mannitol, galactitol, erythritol, maltitol, lactitol, isomalt, maltose, tagatose, lactose, inulin, glycerol, propylene glycol, polyols, polydextrose, fructooligosaccharides, cellulose and cellulose derivatives, and the like, and mixtures thereof. Additionally, granulated sugar (sucrose) or other caloric sweeteners such as crystalline fructose, other carbohydrates, or sugar alcohols can be used as a bulking agent due to their provision of good content uniformity without the addition of significant calories.

In one embodiment, the at least one bulking agent may be a bulking agent described in U.S. Pat. No. 8,993,027.

In one embodiment, the at least one bulking agent may be a bulking agent described in U.S. Pat. No. 6,607,771.

In one embodiment, the at least one bulking agent may be a bulking agent described in U.S. Pat. No. 6,932,982.

In some embodiments, the tabletop sweetener composition may further comprise at least one anti-caking agent. As used herein the phrase “anti-caking agent” and “flow agent” refer to any composition which prevents, reduces, inhibits, or suppresses the at least one sweetener from attaching, binding, or contacting to another sweetener molecule. Alternatively, anti-caking agent may refer to any composition which assists in content uniformity and uniform dissolution. Non-limiting examples of anti-caking agents include cream of tartar, calcium silicate, silicon dioxide, microcrystalline cellulose (Avicel, FMC BioPolymer, Philadelphia, Pa.), and tricalcium phosphate. In one embodiment, the anti-caking agents are present in the tabletop sweetener composition in an amount from about 0.001 to about 3% by weight of the tabletop sweetener composition.

In some embodiments, the sweetener compositions of any of the preceding aspects and embodiments thereof are encapsulated using typical means for encapsulating flavor or fragrance compounds. Non-limiting examples of such technology are set forth in U.S. Patent Application Publication Nos. 2016/0235102, 2019/0082727, 2018/0369777, 2018/0103667, 2016/0346752, 2015/0164117, 2014/0056836, 2012/0027866, 2010/0172945, and 2007/0128234, as well as U.S. Pat. Nos. 7,488,503, 6,416,799, 5,897,897, 5,786,017, 5,603,971, 4,689,235, 4,610,890, 3,704,137, 3,041,180, and 2,809,895. All of the preceding patent publications and patents are hereby incorporated by reference as though set forth herein in their entireties.

### Non-Animal Protein Materials and Products Made Therefrom

Products intended to replace or substitute meat or dairy products often rely on various non-animal-based materials, such as starches and proteins derived from plants, algae, and fungi, to simulate the texture and flavor of meat or dairy. Non-limiting examples of such plant proteins include soy proteins, pea proteins, bean proteins, grain proteins, and the like. Due to compositional differences between such plant-based materials and animal-derived materials, such as a lack of glutamate-containing proteins and glutathione, these products can lack the umami and/or kokumi taste that consumers traditionally associate with meat or dairy products.

Thus, in certain aspects, the disclosure provides a flavored product comprising a plant-based material (such as a plant-based starch, a plant-based protein, or a combination thereof) and an gingerdiol compound (according to any aspects and embodiments set forth herein), or a comestibly acceptable salt thereof. In some further embodiments, the flavored product can include any features of combination of features set forth above for ingestible compositions that contain the gingerdiol compound, or a comestibly acceptable salt thereof. In some embodiments, the flavored product is a beverage, such as soy milk, almond milk, rice milk, oat milk, a protein drink, a meal-replacement drink, or other like product. In some other embodiments, the flavored product is a meat-replacement product, such as a plant-based chicken product (such as a plant-based chicken nugget), a plant-based beef product (such as a plant-based burger), and the like. In some other embodiments, the flavored product is a protein powder, a meal-replacement powder, a plant-based creamer for coffee or tea, and the like. In certain further embodiments, any such products contain additional ingredients, and have additional features, as are typically used in the preparation and/or manufacture of such products. For example, such an gingerdiol compound, or comestibly acceptable salts thereof, may be combined with other flavors and taste modifiers, and may even be encapsulated in certain materials, according to known technologies in the relevant art. Suitable concentrations of the gingerdiol compound, or comestibly acceptable salts thereof, are set forth above.

In some further embodiments analogous to the above embodiments, proteins or starches from algal or fungal sources can be used instead of or in combination with plant starches or proteins.

### Non-Meat Protein Materials and Products Made Therefrom

Certain non-meat animal proteins, such as dairy proteins and proteins from bone broth, are commonly used in food products, and are also sold as the primary ingredient in certain protein powders. Such proteins can impart flavors that lack the full umami or kokumi taste that consumers may desire. This is especially true for protein isolates, such as protein isolates of whey protein, collagen protein, casein proteins, and the like. Thus, the present disclosure provides ingestible compositions that include non-meat animal proteins and the gingerdiol compound (according to any aspects and embodiments set forth herein), or a comestibly acceptable salt thereof. The gingerdiol compound, or its comestibly acceptable salts, can be present in any suitable combination, according to the embodiments set forth in the preceding sections of the present disclosure. In some embodiments, the non-meat animal protein is a bone protein, such as a collagen protein derived from the bones of an animal, such as a cow, pig, donkey, horse, chicken, duck, goat, goose, rabbit, lamb, sheep, buffalo, ostrich, camel, and the like. In some embodiments, the non-meat animal protein is a milk protein, such as a whey protein, a casein protein, or any combination thereof. The milk can be the milk of any suitable animal, such as a cow, donkey, horse, sheep, buffalo, camel, and the like.

The gingerdiol compound, or its comestibly acceptable salts, can also be included in certain food or beverage products that include animal milk or materials derived from animal milk. Such products include cheeses, cheese spreads, yogurt, kefir, milk, processed dairy products, cottage cheese, sour cream, butter, and the like.

### EXAMPLES

### Example 1: Cloning of Oxidoreductases from the Bacillaceae Family

Two protein sequences from bacteria of the Bacillaceae family, in particular the genera Bacillus and Weizmannia with a length of 248 amino acids were identified to examine to determine if they have KRED function. The amino acid sequences of the proteins are provided in SEQ ID NO: 1 and SEQ ID NO: 3.

Their coding nucleotide sequences (SEQ ID NO: 2 and SEQ ID NO: 4), each with a length of 747 bp, derived from the genomic DNA sequences of Bacillus sp. and Weizmannia coagulans respectively, were synthesized in vitro as full-length single gene fragments and cloned into suitable expression vectors, e.g., pET24a (Novagen) or pQE 70 (Qiagen). The terminal restriction sites of the fragments were chosen so that the open reading frame (ORF) of the gene can be read without errors during translation. The cloning took place in pET24a via the cleavage sites of the restriction endonucleases NdeI and HindIII. For the directed cloning, the vector and gene fragment were excised by the endonucleases and linked in a ligation reaction by T4 DNA ligase (New England Biolabs). The resulting plasmid construct in each case was transformed into Escherichia coli Top10F® (Invitrogen), isolated via plasmid extraction (NucleoSpin plasmid EasyPure, Macherey-Nagel) and verified by sequencing.

### Example 2: Heterologous Expression of the Oxidoreductases

For the heterologous expression of the oxidoreductases from Bacillus sp. (SEQ ID NO: 1) or Weizmannia coagulans (SEQ ID NO: 3) in Escherichia coli the expression strains BL21 (DE3) (Invitrogen) or RB791 (K 12, Coli Genetic Stock Center, Yale) were used. Chemically competent cells were transformed with a plasmid construct containing the coding nucleotide sequence. Transformed cells were selected for kanamycin (100 mg/L) or ampicillin (100 mg/mL).

The recombinant clones were cultivated in 600 ml LB medium (1% tryptone, 0.5% yeast extract, 1% NaCl) with 100 mg/L antibiotic at 35° C., 160 rpm in an incubation shaker. During the exponential growth phase, the culture was cooled down to 25° C. when an optical density of 0.3 at 550 nm (OD550) was reached. When an optical density (OD550) of 0.6 was reached, expression was induced by adding 0.1 mM isopropyl β D thiogalactopyranoside (IPTG). After a further 20 hours of incubation at 25° C. and 160 rpm, the cells were harvested. The collected wet cells were stored at −20° C.

### Example 3: Determination of the Enzyme Activities for ethyl-2-oxo-4-phenylbutyrate with Enzyme Lysate

Enzyme lysates (10% [w/v] and 20% [w/v] wet cell mass) were used to determine the enzyme activities. Enzyme lysates were prepared in TEA buffer (100 mM triethanolamine pH 7.0, 2 mM MgCl₂). 0.1 g or 0.2 g wet cell mass was completely resuspended in 1 ml TEA buffer. The cells were disrupted in 2 ml reaction vessels by adding a 0.5 ml corresponding volume of glass beads at 24 Hz for 10 minutes in a vibrating mill (Retsch, MM200). The cell lysate was then sedimented in a tabletop centrifuge at 7500×g for 60 seconds. The cell-free supernatant, the enzyme lysate, was used immediately to determine the activity.

The activity was measured at room temperature in a reaction volume of 1 ml in a semi-micro cuvette (layer thickness 1 cm). The reaction mixture was composed as follows: 100 mM triethanolamine pH 7.0, 2 mM MgCl2, 0.3 mM NADH and 1 mM ethyl-2-oxo-4-phenylbutyrate (CAS No. 64920 29 2)

The reaction was started by adding 15 μL or 25 μL of enzyme lysate diluted in TEA buffer. The individual dilution was chosen for each enzyme lysate in such a way that a linear decrease in absorption at 340 nm could be recorded over a period of 30 seconds (UV-Vis spectrophotometer, Shimadzu). The linear rate of decrease of the absorption per minute divided by the extinction coefficient of NADH (6.23 mM⁻¹ cm⁻¹) is proportional to the enzyme activity in relation to the wet cell mass used. The enzyme activity corresponds to the amount of enzyme that converts one μmol of substrate in one minute (1 U/g=1 μmol min⁻¹ g⁻¹). Each measurement was carried out at least three times and the mean values were determined. For recombinant RB791 cells which contain the oxidoreductase from Bacillus sp. (SEQ ID NO: 1) or Weizmannia coagulans (SEQ ID NO: 3), respectively, enzyme activities of 1825 U/g and 71 U/g were determined.

### Example 4: Characterization of Oxidoreductases with Regard to the Reducing Properties of [6]-Gingerol

The oxidoreductases with the polypeptide sequences SEQ ID NO: 1 and SEQ ID NO: 3 were examined by the following method to determine the conversion rate and diasterioselectivity by reducing [6]-gingerol to the corresponding (3R, 5S)-alcohol:

Recombinant Escherichia coli cells which carry the genes of the oxidoreductases SEQ ID NO: 1 or SEQ ID NO: 3 were cultivated as described in Example 2. To produce an enzyme suspension, 1 g wet cells were completely resuspended with TEA buffer (100 mM triethanolamine pH 7.0, 2 mM MgCl2) in a total volume of 5 mL and then mixed until homogeneous by adding 5 mL glycerol solution (80% [v/v] in TEA buffer). In the reaction setup, 2 mg [6]-gingerol per 50 L of this enzyme suspension were added to 160 μL KPP buffer (100 mM potassium phosphate buffer, pH 7.5, 1 mM MgCl2), together with 0.0035 g NADH and 20 μL 2-propanol. After incubating the samples at 25° C. and 1200 rpm for 24 hours, the reaction solutions were extracted with 1 ml each of methyl tert-butyl acetate and centrifuged. 250 μL of the extract were removed and dried. Then 1 ml acetonitrile was added and analyzed by means of HPLC. A Luna PFP (2) column (150×3 mm) from Phenomenex (Germany) was used for detecting the conversion of [6]-gingerol and the diastereomers formed.

| | SEQ ID NO: 1 | SEQ ID NO: 3 |
|---|---|---|
| Ratio: 3S,5S/3R,5S (%) | 4.5/95.5 | 1.4/98.6 |
| Conversion (%) | >71 | >84 |

### Example 5: Conversion of [6]-Gingerol to the Corresponding (3R, 5S) Gingerdiol on a 0.5 mL Scale with 2-Propanol as Cosubstrate

Recombinant cells of Escherichia coli which carry the oxidoreductase gene SEQ ID NO: 1 were cultured as described in Example 2. To produce an enzyme suspension, 2 g harvested cells were completely resuspended with KPP buffer (100 mM potassium phosphate buffer pH 8.0, 1 mM MgCl2) in a total volume of 6 mL and then mixed until homogeneous by adding 4 mL glycerol. For the reduction of 50 mg 6 gingerol, 50 μL of this enzyme suspension were added to 300 μl KPP buffer (100 mM potassium phosphate buffer, pH 8.0, 1 mM MgCl2), together with 0.05 mg NAD and 150 μL 2-propanol. The reaction solution was shaken at 30° C. and 1200 rpm. After an incubation time of 48 hours, the reaction was stopped and extracted by adding 1 ml of methyl tert-butyl acetate. 100 μL extract were dried, 1 mL acetonitrile was added and analyzed by means of HPLC. A Luna PFP (2) column (150×3 mm) from Phenomenex (Germany) was used to detect the conversion of [6]-gingerol and the diastereomers formed. The measured rate of conversion of [6]-gingerol was >80%, the optical purity of the product was (3S, 5S)/(3R, 5S)=0.4%/99.6%.

### Example 6: Conversion of [6]-Gingerols to the Corresponding (3R, 5S) Gingerdiol on a 0.5 mL Scale with a Formate Dehydrogenase (FDH) as Coenzyme and Sodium Formate as Cosubstrate

Recombinant cells of Escherichia coli which carry the oxidoreductase gene SEQ ID NO: 1 were cultured as described in Example 2. To produce an enzyme suspension, 2 g of harvested cells were completely resuspended with KPP buffer (100 mM potassium phosphate buffer pH 8.0, 1 mM MgCl2) in a total volume of 6 mL and then mixed until homogeneous by adding 4 mL glycerol. For the reduction of 50 mg of [6]-gingerol, 50 μL of this enzyme suspension was added to 362 μL KPP buffer (100 mM potassium phosphate buffer, pH 8.0, 1 mM MgCl2), together with 0.1 mg NAD, 37.5 μL of the FDH suspension (20% [w/v] FDH), and 50 μL of sodium formate solution (50% [w/v]). The reaction solution was shaken at 30° C. and 1200 rpm. After an incubation time of 48 hours, the reaction was stopped by extracting with 1 ml of methyl tert-butyl acetate. 100 μl of extract were dried, mixed with 1 ml of acetonitrile and analyzed by means of HPLC. A Luna PFP (2) column (150×3 mm) from Phenomenex (Germany) was used to detect the conversion of [6]-gingerol and the diastereomers formed. The measured rate of conversion of [6]-gingerol was >93%, the optical purity of the product was (3S, 5S)/(3R, 5S)=0.8%/99.2%.

### Example 7: Biochemical Reduction of High Content [6]-Gingerol

A typical biochemical reduction of [6]-gingerol was done as follows: Into a 1.5 Liter sulfuration flask were added: 25 g of [6]-gingerol (95%), 269.3 mL of 0.2M potassium phosphate pH=6, 49.2 mL of 50% (w/v) sodium formate dissolved in 0.2 M of potassium phosphate pH=6, 49 mL of 6 mg/mL NAD dissolved in 0.2 M of potassium phosphate pH=6, 40.8 mL of 20% (w/v) of spray dried formate dehydrogenase SP22 (Cambrex IEP, Wiesbaden, Germany) dissolved in cold water, 77.5 mL of 15% (w/V) of spray dried ketoreductase IEP Ox95 (Cambrex IEP, Wiesbaden, Germany) dissolved in cold water. The reaction was left at 20° C. under mechanical stirring at 500 rpm (large teflon propeller 5 cm×2.5 cm). Samples were withdrawn for analysis by HPLC in quadruplicates using vanillyl alcohol as the internal standard:

| Reaction | | Concentration | |
|---|---|---|---|
| time | Compound | (g L⁻¹) | Remark |
| 22 h | (R,S)-diol | 40.9 ± 1.0 | de = 99.2 |
| | (S,S)-diol | 0.175 ± 0.005 | |
| | pseudo- | 0.04 | side product |
| | gingerol | | (3-hydroxy-1-(4-hydroxy-3- |
| | | | methoxyphenyl)decan-5-one) |
| | [6]-gingerol | 0.03 | substrate conversion: >99% |

After 22 h the entire reaction was extracted three times with 1 volume (500 ml) of distilled ethyl acetate. Phase separation was by centrifugation at 5000 g for 20 min. The organic phases were combined and washed with 2×700 mL of an aqueous solution containing 8% of NaCl, then dried over anhydrous sodium sulfate. The dried organic phase was evaporated at 40° C. under 7 mbar for 1 hour). A residue of 25.61 g of crude (R,S)-[6]-gingerdiol was obtained containing 88.75% of (R,S)-[6]-gingerdiol and still containing some residual ethyl acetate for avoiding dehydration of the (R,S)-[6]-gingerdiol by excessive heating. Determination of the diastereomeric excess (de) as well as of side products was via the use of available reference substances and mass spectrometric analyses.

Samples taken from the reaction were diluted 20 times with an ethanolic solution containing 1 g L⁻¹ of vanillyl alcohol as the internal standard. The diluted samples were filtered through a PTFE membrane prior to analysis by HPLC. The injection volume was 3 microL. The Agilent 1100 system with DAD detector at 280 nm equipped with a Phenomenex Luna® column 3 μm C18(2) 100 Å, 150×4.6 mm was used. The solvent system consisted of water with 0.1% of formic acid (A) and acetonitrile with 0.1% of formic acid (B). The gradient of solvent B consisted of 10% (0 min), 10% (2 min), 95% (40 min), 95% (41 min), 10% (42 min), 10% (47 min).

### Example 8: Biochemical Reduction of Low Content [6]-Gingerol

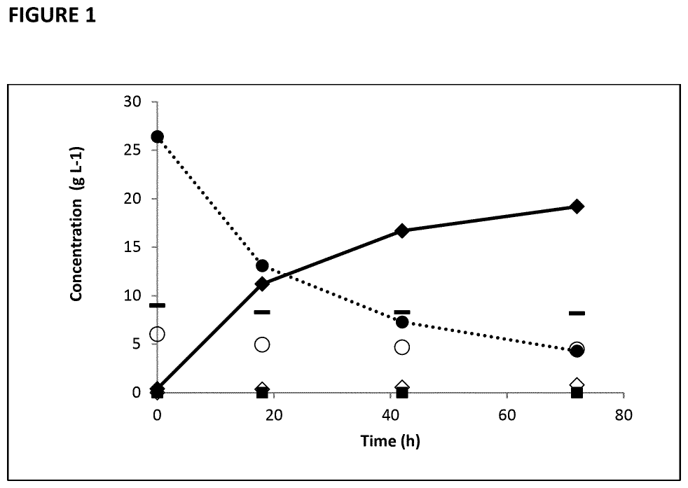

Into an Eppendorf tube were added: 390 microL of potassium phosphate buffer 0.1 M, pH 8, 10 microL of 4 mg mL⁻¹ of NAD, 150 microL of oleoresin containing 32% of [6]-gingerol dissolved in isopropanol, and 71.5 microL of the enzyme IEP Ox95 (Cambrex IEP, Wiesbaden, Germany). Tubes were inserted into Eppendorf's Thermomixer run at 1400 rpm at 24° C. for up to 3 days. Reactions were diluted in 12 mL of ethanol containing 1 g L⁻¹ of vanillyl alcohol as the internal standard prior to the analysis by HPLC-UV as described. The results are shown in FIG. 1.

### Example 9: Non-Catalyzed Esterification

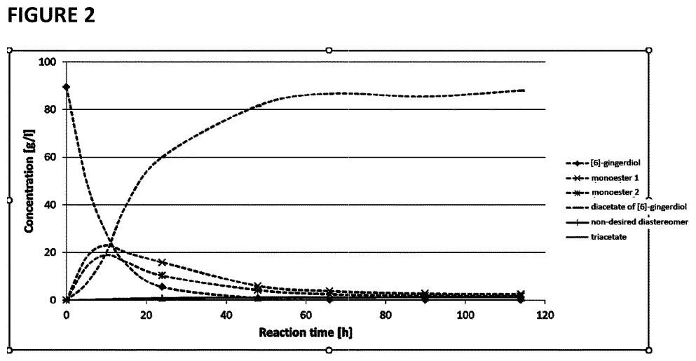

A typical esterification of [6]-gingerdiol was done as follows. Into a 500 mL flask were added: 22.1 g of 6-Gingerdiol and 198 g of acetic acid. The reaction was allowed to react for 114 h at 100° C. under magnetic stirring at 500 rpm. Samples were withdrawn over time for analysis by HPLC using vanillyl alcohol as the internal standard. The results are shown in FIG. 2.

Virtually complete conversion of the substrate (99.9%) was observed and reaction equilibrium was greatly shifted toward the target compound. Mono-acetates (on the aliphatic side chain only) were still present and represented 5.0% of the 6-gingerdiol derivatives detected, while [6]-gingerdioldiacetate represented around 92%. The non-desired dia-stereoisomer of [6]-gingerdioldiacetate was not detected. The tri-acetate compound represented below 2%. Yield of [6]-gingerdioldiacetate was in the range of 73.7%. The excess acetic acid was then evaporated to yield 38.3 g of a viscous, dark residue. Short-path distillation (200° C., 0.8 mbar) of this residue afforded 23.54 g of a pale yellow oil as a distillate with a non-optimized molar yield of 85%.

| | End of reaction | | Crude concentrate | | Short-path distillate | |
|---|---|---|---|---|---|---|
| | w | Distribution | w | Distribution | w | Distribution |
| [6]-gingerdiol | 0.0% | 0.1% | 0.0% | 0.0% | 0.0% | 0.0% |
| Mono-esters | 0.5% | 4.9% | 2.9% | 4.9% | 3.9% | 5% |
| [6]-Gingerdiol- | 11.2% | 93.6% | 61.8% | 93.6% | 82.8% | 93% |
| diacetate | | | | | | |
| Tri-ester | 0.2% | 1.4% | 1.0% | 1.5% | 1.3% | 2% |
| Diastereomer | 0.0% | 0.0% | 0.0% | 0.0% | 0.0% | 0.0% |
| Total | 11.9% | 100.0% | 65.6% | 100.0% | 88.0% | 100.0% |

### Example 10: Non-Catalyzed Esterification at Boiling Temperature

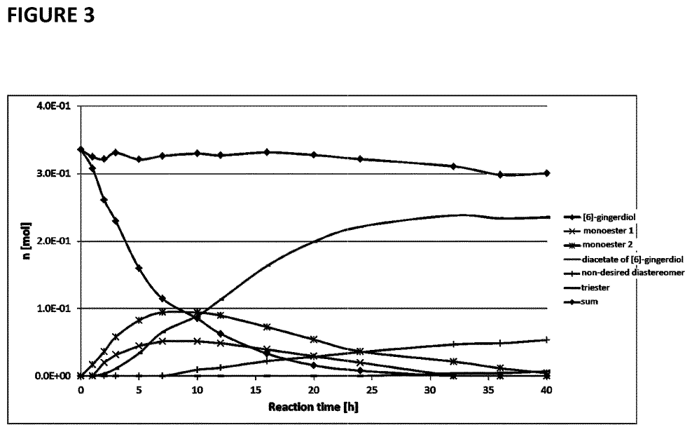

The esterification process of 6-gingerdiol was done as follows. Into a 2 L flask were added: 98.6 g of 6-Gingerdiol (97%) and 893 g of acetic acid. The reaction was allowed to react for 40 h at 118° C. under magnetic stirring at 500 rpm. The formed water and some acetic acid were distilled during the course of the reaction. Samples of the reaction mixture were withdrawn over time for analysis by HPLC using vanillyl alcohol as the internal standard. The result is shown in FIG. 3.

Complete conversion of the substrate was observed and reaction equilibrium was greatly shifted toward the target compound. Mono-acetates esters (on the aliphatic side chain only) were still present and represented 1.2% of all 6-gingerdiol derivatives detected, while [6]-gingerdioldiacetate represented 78.3%. The (S,S)-diastereoisomer of [6]-gingerdioldiacetate was also detected at 18.1%. The triacetate compound could be detected at 2.4%. Yield of [6]-gingerdioldiacetate was in the range of 68%. The excess acetic acid was then evaporated to yield 125.8 g of a viscous, dark residue. Short-path distillation (200° C., 0.8 mbar) of 20 g of this residue afforded 15.44 g of a pale yellow oil as a distillate with a molar yield of 96%.

| | End of reaction | | Crude concentrate | | Short-path distillate | |
|---|---|---|---|---|---|---|
| | w | Distribution | w | Distribution | w | Distribution |
| Mono-esters | 0.2% | 1.2% | 0.8% | 1.1% | 1.0% | 2.1% |
| [6]-Gingerdiol- | 15.2% | 78.3% | 64.6% | 78.5% | 79.3% | 78.8% |
| diacetate | | | | | | |
| (S,S)-Diastereomer | 3.5% | 18.1% | 14.9% | 18.1% | 16.4% | 16.3% |
| Tri-ester | 0.5% | 2.4% | 2.1% | 2.3% | 3.1% | 2.8% |
| Total | 19.5% | 100.0% | 82.5% | 100.0% | 99.8% | 100.0% |

### Example 11a: Acid Catalyzed Esterification

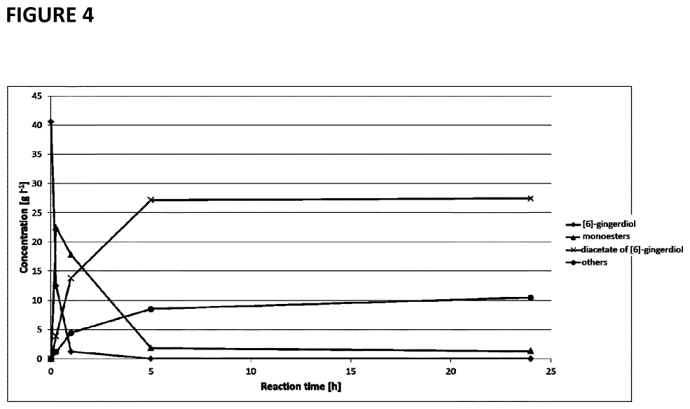

Into a 15 ml test tube were added: 1 g of crude [6]-gingerdiol residue containing 54% w/w of (R,S)-[6]-gingerdiol (3.37 mmol) was dissolved, 9 g of acetic acid (150 mmol), and 25 mg of p-TSA (ca. 80 mmol/mol of substrate) to catalyze the reaction. Tubes were inserted into Eppendorf's Thermomixer run at 750 rpm at 99° C. for 24 h. The reaction mixture turned from pale yellow to dark brown, indicating that side reactions like polymerization occurred; however such structures could not be detected. Samples were withdrawn over time for analysis by HPLC using vanillyl alcohol as the internal standard. The result is shown in FIG. 4.

For these reactions, the excess acetic acid was typically evaporated and the viscous dark residue was purified by flash chromatography (solid disposal method, SiO₂ 30 μm, heptane: EtOAc 6:4).

### Example 11b: Acid Catalyzed Esterification

Into a 15 ml test tube were added: 2.99 g of purified [6]-gingerdiol containing 97% w/w of (R,S)-[6]-gingerdiol (9.8 mmol) was dissolved, 1.30 g of acetic acid (21.4 mmol), and 19.7 mg of p-TSA (ca. 11.7 mmol/mol of substrate) to catalyze the reaction. Tubes were inserted into Eppendorf's Thermomixer run at 750 rpm at 99° C. for 24 h. The reaction mixture turned from pale yellow to dark brown, indicating that side reactions like polymerization occurred; however such structures could not be detected. Samples were withdrawn over time for analysis by HPLC using vanillyl alcohol as the internal standard.

| | 0.5 h of reaction | | 2 h of reaction | | 8 h of reaction | | 24 h of reaction | |
|---|---|---|---|---|---|---|---|---|
| | w | Distribution | w | Distribution | w | Distribution | w | Distribution |
| [6]-gingerdiol | 40.6% | 65.0% | 21.3% | 33.3% | 17.4% | 27.1% | 16.7% | 26.0% |
| Mono-esters | 20.3% | 32.5% | 32.0% | 50.1% | 23.4% | 36.3% | 21.9% | 34.1% |
| [6]-gingerdiol- | 1.3% | 2.0% | 9.9% | 15.5% | 22.1% | 34.3% | 22.5% | 35.0% |
| diacetate | | | | | | | | |
| Tri-ester | 0.3% | 0.4% | 0.7% | 1.1% | 1.5% | 2.3% | 3.1% | 4.9% |
| Total | 62.5% | 100.0% | 63.9% | 100.0% | 64.5% | 100.0% | 64.2% | 100.0% |

### Example 12: Acid Catalyzed Acetylation

Into a 15 ml test tube were added: 3.04 g of purified [6]-gingerdiol containing 97% w/w of (R,S)-[6]-gingerdiol (10.26 mmol) was dissolved, 2.23 g of acetic acid anhydride (21.19 mmol), and 16.0 mg of p-TSA (ca. 9.1 mmol/mol of substrate) to catalyze the reaction. Tubes were inserted into Eppendorf's Thermomixer run at 750 rpm at 99° C. for 7 h. The reaction mixture turned from pale yellow to dark brown, indicating that side reactions like polymerization occurred; however such structures could not be detected. After 7 h of reaction time, an additional 2.23 g of acetic acid anhydride (21.19 mmol) was added into the test tube, which was inserted into Eppendorf's Thermomixer and allowed to run at 750 rpm at 99° C. for a total reaction time of 50 h. Samples were withdrawn over time for analysis by HPLC using vanillyl alcohol as the internal standard.

| | | | | | 7.5 h of reaction | | 50 h of reaction | |
|---|---|---|---|---|---|---|---|---|
| | 0.25 h of reaction | | 7 h of reaction | | (0.5 h after addition) | | (43 h after addition) | |
| | w | Distribution | w | Distribution | w | Distribution | w | Distribution |
| [6]-gingerdiol | 2.9% | 7.1% | 3.1% | 6.9% | 0.1% | 0.3% | 0.0% | 0.0% |
| Mono-esters | 7.8% | 18.8% | 4.5% | 10.4% | 2.5% | 10.9% | 0.1% | 0.3% |
| [6]-gingerdiol- | 14.5% | 35.2% | 19.0% | 42.4% | 0.4% | 1.7% | 0.0% | 0.1% |
| diacetate | | | | | | | | |
| Tri-ester | 16.0% | 38.9% | 17.5% | 40.3% | 20.1% | 87.1% | 23.3% | 99.6% |
| Total | 41.2% | 100.0% | 44.1% | 100.0% | 23.1% | 100.0% | 23.4% | 100.0% |

SEQUENCE LISTING

SEQ ID NO:1
Met Ser Gly Arg Val Ala Val Ile Thr Gly Ala Gly Ser Gly Ile Gly
Arg Glu Thr Cys Leu Thr Phe Ala Arg Lys Gly Glu Lys Val Val Val
Ala Asp Ile Asn Glu Glu Lys Gly Leu Glu Thr Thr Glu Leu Val Lys
Ala Glu Gly Gly Glu Ala Val Phe Val Asn Thr Asp Val Ser Lys Phe
Glu Glu Val Glu Ala Leu Val Asp Gln Ala Val Glu Thr Tyr Gly Ser
Ile Asp Ile Met Phe Asn Asn Ala Gly Ile Gly Lys Met Gly His Val
Leu Ser Leu Asn Ile Glu Asp Tyr Leu His Val Ile Asp Val Asn Gln
His Gly Val Ala Tyr Gly Ile Ile Ala Ala Gly Arg Lys Met Arg Asp
Leu Asp Ile Lys Gly Val Ile Ile Asn Ser Ala Ser Val Phe Gly Tyr
Leu Ala Asn Phe Gly Thr Phe Pro Tyr Gln Ala Ser Lys Gly Ala Val
Arg Met Met Thr Gln Asn Ala Ala Leu Glu Leu Ser Gln Tyr Gly Ile
Arg Val Val Gly Ile Ala Pro Gly Ser Val Asp Thr Ser Ile Ile Gln
Ala Tyr Lys Asp Ala Gly Met Thr Gln Lys Met Lys Glu Gln Gln Met
Arg Lys Glu Leu Ile Arg Pro Glu Ala Ile Ala Asn Ala Val Tyr Leu
Leu Thr Leu Glu Glu Ala Asp Val Ile Asn Gly Ser Val Val Met Leu
Asp Asp Gly Tyr Ala Ser Phe Lys
SEQ ID NO: 2

| | |
|---|---|
| atgtcaggca gagtagcagt cattacaggc gccggcagcg ggataggccg ggaaacatgt | 60 |
| ttaacatttg cgcgcaaagg cgaaaaagtc gtcgttgctg atattaatga agaaaaaggg | 120 |
| cttgaaacga ccgagctggt caaagcggaa ggcggagaag ctgtttttgt caatacagat | 180 |
| gtttcgaaat ttgaagaagt ggaagcgctt gttgaccagg ctgtggaaac gtacggatct | 240 |
| atagatatta tgtttaataa cgccgggatc gggaagatgg gacatgtcct gagtttaaat | 300 |
| atagaagatt atttgcacgt gattgatgtc aatcagcacg gagtggcgta cggcattatt | 360 |
| gctgccggca ggaaaatgcg tgaccttgat ataaaaggtg tgatcatcaa ttccgcttct | 420 |
| gttttcggat atttggccaa tttcgggacg ttcccttacc aagcttccaa aggcgcggtc | 480 |
| cggatgatga cccaaaacgc ggcattggaa ctttcccaat acggcatccg ggtagtcggc | 540 |
| attgccccgg gcagcgtgga tacgtcgatt atccaggcct ataaagatgc cggaatgacg | 600 |
| cagaaaatga aagaacagca aatgcggaaa gaattgatcc ggccggaagc aatcgccaat | 660 |
| gcagtttacc tgctgaccct cgaagaagcg gatgtcatca atggcagcgt tgttatgctc | 720 |
| gatgacggct atgcatcgtt taaataa | 747 |

SEQ ID NO: 3
Met Ser Gly Arg Val Ala Val Ile Thr Gly Ala Gly Ser Gly Ile Gly
Trp Glu Thr Ser Leu Thr Phe Ala Arg Lys Gly Asp Ser Val Val Val
Ala Asp Ile Asp Glu Glu Lys Gly Leu Glu Thr Val Glu Leu Ile Lys
Gln Glu Gly Gly Asp Ala Val Phe Ala Lys Thr Asp Val Ser Lys Phe
Glu Glu Val Glu Ser Leu Val Asn Gln Ala Val Glu Ile Tyr Gly Thr
Ile Asp Val Met Phe Asn Asn Ala Gly Ile Gly Thr Leu Gly His Ile
Leu Ser Lys Lys Ile Glu Asp Tyr Leu Arg Val Ile Asp Val Asn Gln
His Gly Val Ala Tyr Gly Ile Ile Ala Ala Gly Arg Lys Met Arg Glu
Leu Asp Val Lys Gly Val Ile Ile Asn Thr Ala Ser Val Phe Gly Tyr
Leu Ala Asn Phe Gly Thr Phe Pro Tyr Gln Ala Ala Lys Gly Ala Val
Arg Met Met Thr Gln Asn Ala Ala Leu Glu Leu Ala Gln Tyr Gly Ile
Arg Val Val Gly Ile Ala Pro Gly Cys Val Asp Thr Pro Ile Ile Gln
Ala Tyr Lys Asp Ala Gly Met Thr Gln Lys Met Thr Glu Gln Gln Met
Arg Lys Lys Leu Ile Arg Pro Glu Ala Ile Ala Asn Ala Val Tyr Leu
Leu Thr Leu Glu Glu Ala Asp Ala Ile Asn Gly Ser Val Val Met Leu
Asp Asp Gly Tyr Ala Ser Phe Lys
SEQ ID NO: 4

| | |
|---|---|
| atgtcaggca gagttgcagt gattacaggg gccggcagcg ggattggctg ggaaacaagt | 60 |
| ttaacttttg cacgtaaagg cgattccgtt gtggttgccg atattgatga agaaaaaggg | 120 |
| cttgaaacgg tggaattgat caaacaagaa ggcggagatg ctgtttttgc caagacggat | 180 |
| gtttccaaat ttgaagaagt cgaatcactt gtcaatcagg cagttgaaat atacgggacg | 240 |
| attgacgtga tgttcaacaa cgccggaatc ggaacgctcg ggcatatcct gagtaagaaa | 300 |
| atagaagatt acttgcgtgt cattgatgtc aatcagcacg gcgtggcata cgggatcatt | 360 |
| gctgcaggca gaaaaatgcg cgagctggat gtaaaaggcg tcattatcaa cacggcttcc | 420 |
| gtattcggtt atttggcaaa tttcggtact ttcccatatc aggctgcaaa aggcgctgtc | 480 |
| cggatgatga cgcaaaatgc cgcgctcgaa ctggcacaat acggcattcg cgtcgtcggc | 540 |
| attgcgccgg gttgtgtgga taccccgatt atacaagctt ataaagatgc cggaatgacg | 600 |
| caaaaaatga cagaacagca aatgcgcaaa aaactgatcc gcccggaagc gatcgcaaat | 660 |
| gcggtttatc tgttaacgct tgaagaagca gatgctatta acggaagcgt tgtcatgctt | 720 |
| gatgacggct atgcttcttt caaataa | 747 |

1. A process for making a compound of formula (III): 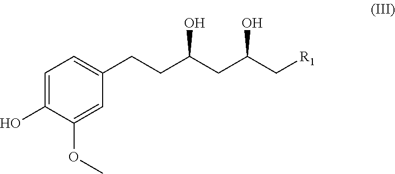
in the form of a mixture or salt thereof; wherein: R₁ is a hydrogen atom or a C₁₋₂₀ alkyl group, the process comprising reducing a precursor compound of formula (II) 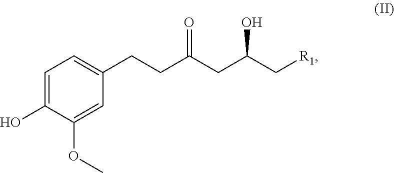
in the form of a mixture or salt thereof, wherein R₁ is defined as in formula (III), with a oxidoreductase enzyme to form the compound of formula (III).

2. The process of claim 1 wherein the oxidoreductase enzyme is a ketoreductase (KRED) enzyme.

3. The process of claim 2 wherein the KRED enzyme has at least 50%, 55%, 60%, 65%, 70%, 75%, 80%, 85%, 90%, 95%, 96%, 97%, 98% or 99% sequence identity to SEQ ID NO: 1 or SEQ ID NO: 3 or comprises the amino acid sequence of SEQ ID NO: 1 or SEQ ID NO: 3, or is the KRED enzyme IEP Ox95.

4. The process of claim 1, wherein the reduction is a ketoreductase reduction performed in the presence of a co-factor.

5. The process of claim 4 wherein the ketoreductase reduction is performed in the presence of a co-factor regeneration system.

6. The process of claim 5 wherein the co-factor regeneration system is a secondary alcohol, formate dehydrogenase (FDH) or glucose dehydrogenase (GDH) based system.

7. The process of claim 1, wherein the precursor compound of formula (II) is [6]-gingerol, [8]-gingerol, or [10]-gingerol.

8. The process of claim 1, wherein the compound of formula (III) is [6]-gingerdiol, [8]-gingerdiol, or [10]-gingerdiol.

9. The process of claim 1, wherein the process further comprises esterifying the compound of formula (III) to form a compound of formula (I), 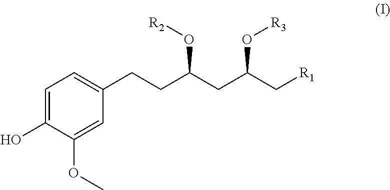
in the form of a mixture or salt thereof; wherein:
R₁ is a hydrogen atom or a C₁₋₂₀ alkyl group; and
each R₂ and R₃, are, independently from each other, —C(O)—H or —C(O)—(C₁₋₄ alkyl).

10. The process of claim 9 wherein the esterification of the compound of formula (III) to form a compound of formula (I) comprises using acetic acid.

11. A process for making a compound of formula (I) 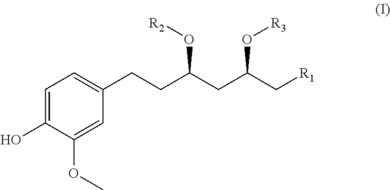
in the form of a mixture or salt thereof; wherein:
R₁ is a hydrogen atom or a C₁₋₂₀ alkyl group; and
each R₂ and R₃, are, independently from each other, —C(O)—H or —C(O)—(C₁₋₄ alkyl),
the process comprising reducing a precursor compound of formula (II) 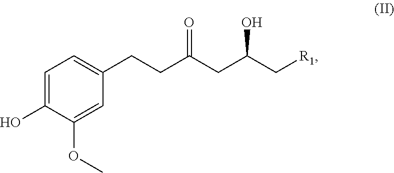
in the form of a mixture or salt thereof, wherein R₁ is defined as in formula (I),
with a oxidoreductase enzyme to form a compound of formula (III), 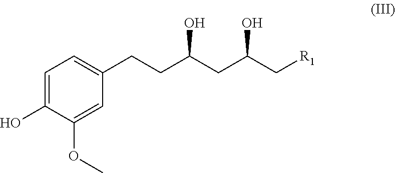
in the form of a mixture or salt thereof; wherein: R₁ is defined as in formula (I),
and esterifying the compound of formula (III) to form the compound of formula (I).

12. A compound of formula (I) obtained by the process of claim 11.

13. (canceled)

14. (canceled)

15. A method of enhancing a salty taste or an umami taste of a flavored product, the method comprising introducing to the food product the compound of formula (I) of claim 12.

16. The method of claim 15 wherein the flavored product is a food product, a beverage product, or an oral care product.

17. A comestible composition for enhancing a salty taste or an umami taste in a flavored product, the composition comprising the compound of formula (I) of claim 12.

18. A flavored product, which comprises the comestible composition of claim 17.

19. The flavored product of claim 18, wherein the flavored product is a food product, a beverage product, or an oral care product.

20. The process of claim 1, wherein the reduction is a ketoreductase reduction performed in the presence of a co-factor, wherein the co-factor is NAD(P)H or NAD(P).

21. The process of claim 1, wherein the precursor compound of formula (II) is [6]-gingerol.

22. The process of claim 1, wherein the compound of formula (III) is [6]-gingerdiol.


SEQUENCE LISTING (4 sequences)

SEQ ID NO: 1 - protein, 248 aa, Bacillus sp.
MSGRVAVITGAGSGIGRETCLTFARKGEKVVVADINEEKGLETTELVKAEGGEAVFVNTDVSKFEEVEALVDQAVETYGSIDIMFNNAGIGKMGHVLSLNIEDYLHVIDVNQHGVAYGIIAAGRKMRDLDIKGVIINSASVFGYLANFGTFPYQASKGAVRMMTQNAALELSQYGIRVVGIAPGSVDTSIIQAYKDAGMTQKMKEQQMRKELIRPEAIANAVYLLTLEEADVINGSVVMLDDGYASFK

SEQ ID NO: 2 - DNA, 747 bp, Bacillus sp.
atgtcaggcagagtagcagtcattacaggcgccggcagcgggataggccgggaaacatgtttaacatttgcgcgcaaaggcgaaaaagtcgtcgttgctgatattaatgaagaaaaagggcttgaaacgaccgagctggtcaaagcggaaggcggagaagctgtttttgtcaatacagatgtttcgaaatttgaagaagtggaagcgcttgttgaccaggctgtggaaacgtacggatctatagatattatgtttaataacgccgggatcgggaagatgggacatgtcctgagtttaaatatagaagattatttgcacgtgattgatgtcaatcagcacggagtggcgtacggcattattgctgccggcaggaaaatgcgtgaccttgatataaaaggtgtgatcatcaattccgcttctgttttcggatatttggccaatttcgggacgttcccttaccaagcttccaaaggcgcggtccggatgatgacccaaaacgcggcattggaactttcccaatacggcatccgggtagtcggcattgccccgggcagcgtggatacgtcgattatccaggcctataaagatgccggaatgacgcagaaaatgaaagaacagcaaatgcggaaagaattgatccggccggaagcaatcgccaatgcagtttacctgctgaccctcgaagaagcggatgtcatcaatggcagcgttgttatgctcgatgacggctatgcatcgtttaaataa

SEQ ID NO: 3 - protein, 248 aa, Weizmannia coagulans
MSGRVAVITGAGSGIGWETSLTFARKGDSVVVADIDEEKGLETVELIKQEGGDAVFAKTDVSKFEEVESLVNQAVEIYGTIDVMFNNAGIGTLGHILSKKIEDYLRVIDVNQHGVAYGIIAAGRKMRELDVKGVIINTASVFGYLANFGTFPYQAAKGAVRMMTQNAALELAQYGIRVVGIAPGCVDTPIIQAYKDAGMTQKMTEQQMRKKLIRPEAIANAVYLLTLEEADAINGSVVMLDDGYASFK

SEQ ID NO: 4 - DNA, 747 bp, Weizmannia coagulans
atgtcaggcagagttgcagtgattacaggggccggcagcgggattggctgggaaacaagtttaacttttgcacgtaaaggcgattccgttgtggttgccgatattgatgaagaaaaagggcttgaaacggtggaattgatcaaacaagaaggcggagatgctgtttttgccaagacggatgtttccaaatttgaagaagtcgaatcacttgtcaatcaggcagttgaaatatacgggacgattgacgtgatgttcaacaacgccggaatcggaacgctcgggcatatcctgagtaagaaaatagaagattacttgcgtgtcattgatgtcaatcagcacggcgtggcatacgggatcattgctgcaggcagaaaaatgcgcgagctggatgtaaaaggcgtcattatcaacacggcttccgtattcggttatttggcaaatttcggtactttcccatatcaggctgcaaaaggcgctgtccggatgatgacgcaaaatgccgcgctcgaactggcacaatacggcattcgcgtcgtcggcattgcgccgggttgtgtggataccccgattatacaagcttataaagatgccggaatgacgcaaaaaatgacagaacagcaaatgcgcaaaaaactgatccgcccggaagcgatcgcaaatgcggtttatctgttaacgcttgaagaagcagatgctattaacggaagcgttgtcatgcttgatgacggctatgcttctttcaaataa